(19) United States
(12) Patent Application Publication
(10) Pub. No.: US 2024/0423620 A1
(43) Pub. Date: Dec. 26, 2024

(54) COMPRESSIBLE SURGICAL ADJUNCTS, CARTRIDGES, AND CARTRIDGE ASSEMBLIES AND METHODS OF MAKING SURGICAL ADJUNCTS

(71) Applicant: CILAG GMBH INTERNATIONAL, Zug (CH)

(72) Inventors: Peyton Hopson, Cincinnati, OH (US); Madelaine Franzoni, Cincinnati, OH (US); Brody Frost, Fairfield, OH (US); Omar McGiveron, Cincinnati, OH (US); Vincent Hembrick-Holloman, Cincinnati, OH (US); Sarah Scully, Cincinnati, OH (US); Michael J. Vendely, Lebanon, OH (US); Christopher Q. Seow, Cincinnati, OH (US); Ruben Salazar, Cincinnati, OH (US); David Bruns, Cincinnati, OH (US); Andreas Ward, Cincinnati, OH (US); Heather Strang, Cincinnati, OH (US); Jason Harris, Cincinnati, OH (US)

(21) Appl. No.: 18/485,210

(22) Filed: Oct. 11, 2023

Related U.S. Application Data
(60) Provisional application No. 63/522,660, filed on Jun. 22, 2023.

(51) Int. Cl. A61B 17/072; A61L 31/06; A61L 31/10; A61L 31/12; A61L 31/14

(52) U.S. Cl. CPC A61B 17/07292; A61L 31/06; A61L 31/10; A61L 31/129; A61L 31/148; A61B 2017/00526; A61L 2420/02

(57) ABSTRACT

The disclosed technology includes a surgical adjunct that includes bioabsorbable material such as a polyurethane foam. A volumetric ratio of the polyurethane foam to the total volume of the surgical adjunct may be in a range of about 0.125 to about 0.325. A glass transition temperature of the surgical adjunct may be about 0° C. to about 40° C.

### CROSS-REFERENCE TO RELATED APPLICATIONS

This application claims priority under 35 U.S.C. § 119 to U.S. Provisional Patent Application No. 63/522,660, filed Jun. 22, 2023, the entire contents of which are fully incorporated herein by reference.

This application is related to U.S. patent application Ser. Nos. 18/484,807, 18/484,929, 18/485,161, 18/485,145, 18/485,117, 18/485,083, 18/485,047, 18/484,988, all filed on Oct. 11, 2023, the entire contents of each of which are fully incorporated herein by reference.

### FIELD

The present invention relates generally to compressible surgical adjuncts, cartridges, cartridge assemblies and methods of making adjuncts and cartridge assemblies.

### BACKGROUND

Surgical staplers are used in surgical procedures to close openings in tissue, blood vessels, ducts, shunts, or other objects or body parts involved in the particular procedure. The openings can be naturally occurring, such as passageways in blood vessels or an internal organ like the stomach, or they can be formed by the surgeon during a surgical procedure, such as by puncturing tissue or blood vessels to form a bypass or an anastomosis, or by cutting tissue during a stapling procedure.

Most staplers have a handle (some of which are directly user operable, others of which are operable by a user via a robotic interface) with an elongate shaft extending from the handle and having a pair of movable opposed jaws formed on an end thereof for holding and forming staples therebetween. The staples are typically contained in a staple cartridge, which can house multiple rows of staples and is often disposed in one of the two jaws for ejection of the staples to the surgical site. In use, the jaws are positioned so that the object to be stapled is disposed between the jaws, and staples are ejected and formed when the jaws are closed, and the device is actuated. Some staplers include a knife configured to travel between rows of staples in the staple cartridge to longitudinally cut and/or open the stapled tissue between the stapled rows.

### SUMMARY

There is provided, in accordance with an example of the present invention, a surgical adjunct that includes a polyurethane foam. A volumetric ratio of the polyurethane foam to the total volume of the surgical adjunct is in a range of about 0.125 to about 0.325. A glass transition temperature of the surgical adjunct is about 0° C. to about 40° C.

There is provided, in accordance with an example of the present invention, a method of making a surgical adjunct. The method includes selectively adding a plasticizer to a polyurethane foam to generate the surgical adjunct. The surgical adjunct has a glass transition temperature of about 0° C. to about 40° C.

There is provided, in accordance with an example of the present invention, a surgical adjunct that includes a polyurethane. A glass transition temperature of the surgical adjunct 604 is about 0° C. to about 40° C.

There is provided, in accordance with an example of the present invention, a surgical staple cartridge assembly, including a cartridge having a length of about 80 mm to about 90 mm and a width of about 8.9 mm to about 14 mm. The surgical staple cartridge assembly also includes a surgical adjunct disposed on the cartridge. The surgical adjunct includes a polyurethane foam comprising a volumetric ratio of the polyurethane foam to the total volume of the surgical adjunct 604 is in a range of about 0.125 to about 0.325 and a plasticizer added to the polyurethane foam. A glass transition T_{g} temperature of the surgical adjunct is about 0° C. to about 40° C. The surgical adjunct having a length of about 40 mm to about 80 mm, a width of about 8 mm to about 12 mm; and a height of about 2.5 mm to about 3.5 mm.

### BRIEF DESCRIPTION OF THE DRAWINGS

This invention will be more fully understood from the following detailed description taken in conjunction with the accompanying drawings, in which:

FIG. 1 is a perspective view of one exemplary embodiment of a conventional surgical stapling and severing instrument;

FIG. 2A is a top view of a staple cartridge for use with the surgical stapling and severing instrument of FIG. 1;

FIG. 2B is a side view of the staple cartridge of FIG. 2A;

FIG. 3 is a side view of a staple in an unfired (pre-deployed) configuration that can be disposed within the staple cartridge of the surgical cartridge assembly of FIG. 2A;

FIG. 4 is a perspective view of a knife and firing bar (“E-beam”) of the surgical stapling and severing instrument of FIG. 1;

FIG. 5 is a perspective view of a wedge sled of a staple cartridge of the surgical stapling and severing instrument of FIG. 1;

FIG. 6A is a longitudinal cross-sectional view of an exemplary surgical cartridge assembly having a compressible non-fibrous adjunct attached to a top or deck surface of a staple cartridge;

FIG. 6B is a longitudinal cross-sectional view of a surgical end effector having an anvil pivotably coupled to an elongate channel and the surgical cartridge assembly of FIG. 6A disposed within and coupled to the elongate channel, showing the anvil in a closed position without any tissue between the anvil and the adjunct;

FIG. 7A is a partial-schematic illustrating the adjunct of FIGS. 6A-6B in a tissue deployed condition;

FIG. 7B is a diagram showing an enlarged portion of an exemplary adjunct with a porous structure;

FIG. 8 is a perspective view of an exemplary cartridge assembly;

FIG. 9A is a side view of an exemplary end effector having an adjunct of bioabsorbable material in a delivery configuration;

FIG. 9B is a side view of an exemplary end effector having an adjunct of bioabsorbable material after firing and release from cartridge;

FIGS. 9C and 9D illustrate example reversible transformations from linear polymers to complex architectures;

FIG. 10A is a top perspective view of an exemplary adjunct after use;

FIG. 10B is a side view of an exemplary adjunct after use;

FIG. 11 is a flow chart showing an exemplary method of forming a surgical adjunct with spatial control of mechanical properties in bioabsorbable material;

FIGS. 12A-12B are flow charts showing exemplary methods of forming a surgical adjunct with tunable absorption pathways in bioabsorbable materials;

FIGS. 13A through 13D are perspective views of exemplary surgical cartridge assemblies having an adjunct of bioabsorbable material attached to a top or deck surface of a staple cartridge;

FIG. 14 is a flow chart showing an exemplary method of forming a surgical adjunct with customizable crosslink density for tuning mechanical properties in bioabsorbable material;

FIG. 15A is a perspective view of an exemplary surgical end effector having a channel and a surgical cartridge with a sled and drivers;

FIG. 15B is a perspective view of an exemplary sled for an exemplary surgical cartridge;

FIG. 15C is a perspective view of an exemplary single driver for an exemplary surgical cartridge;

FIG. 15D is a front view of the exemplary single driver of FIG. 15C;

FIG. 15E is a perspective view of an exemplary double driver for an exemplary surgical cartridge;

FIG. 15F is a front view of an exemplary double driver of FIG. 15E;

FIG. 15G is a cutaway view of an exemplary cartridge in an exemplary end effector;

FIG. 16A is a side view of an exemplary adjunct for a cartridge assembly;

FIG. 16B is a top view of an exemplary adjunct for a cartridge assembly;

FIG. 16C is a front view of an exemplary adjunct for a cartridge assembly;

FIG. 17A is a side view of an exemplary adjunct compressed in a dog bone profile;

FIG. 17B is a front view of FIG. 17A;

FIG. 18A is a perspective view of an exemplary cartridge deck with three levels;

FIG. 18B a perspective view of an enlarged front portion of exemplary cartridge deck of FIG. 18A;

FIG. 18C is a perspective view of an enlarged rear portion of exemplary cartridge deck of FIG. 18A;

FIG. 18D is a top view of an enlarged front portion of exemplary cartridge deck of FIG. 18A;

FIG. 19A is a perspective view of an exemplary cartridge deck with two levels;

FIG. 19B is a perspective view of an enlarged front portion of exemplary cartridge deck of FIG. 19A;

FIG. 19C is a perspective view of an enlarged rear portion of exemplary cartridge deck of FIG. 19A;

FIG. 19D a top view of an enlarged front portion of exemplary cartridge deck of FIG. 19A;

FIG. 20 is graph showing the glass transition temperature T_{g} of an exemplary adjunct;

FIG. 21 is a flow chart showing an exemplary method for making an adjunct;

FIGS. 22A and 22B are flow charts showing exemplary methods for embedding medical additives into a bioabsorbable material;

FIG. 23A is a diagram showing an exemplary adjunct for a cartridge assembly with a hydrophobic coating;

FIG. 23B is a diagram showing an exemplary adjunct for a cartridge assembly with a pattern creating one or more hydrophobic surfaces;

FIG. 23C is a diagram showing an exemplary adjunct for a cartridge assembly with a gradient porosity;

FIG. 23D is a diagram showing an exemplary adjunct for a cartridge assembly with directional porosity;

FIG. 23E is a diagram showing an exemplary adjunct for a cartridge assembly with directional and closed-cell porosity;

FIG. 23F is a diagram showing an exemplary adjunct for a cartridge assembly with a close cell porosity and hydrophobic film;

FIG. 23G is a diagram showing an exemplary adjunct for a cartridge assembly with a hydrophobic section;

FIG. 23H is a diagram showing an exemplary adjunct for a cartridge assembly with a hydrophobic matrix;

FIG. 23I is a diagram showing an exemplary adjunct for a cartridge assembly with an interlaced hydrophobic material;

FIG. 24A is a diagram showing an exemplary adjunct transmurally placed within a patient;

FIGS. 24B and 24C are diagrams showing an exemplary adjunct transmurally placed within a patient;

FIG. 25A is a side view of an exemplary surgical adjunct with a film;

FIG. 25B is a side view of an exemplary surgical adjunct with a film;

FIG. 25C shows an exemplary cartridge assembly including a surgical adjunct with a film;

FIG. 26A is a diagram showing an exemplary adjunct for a cartridge assembly with a double gradient of pore diameters;

FIG. 26B is a diagram showing an exemplary adjunct for a cartridge assembly fabricated from two exemplary surgical adjuncts each with a gradient of pore diameters;

FIG. 27A shows an exemplary cartridge assembly with a post and an adhesive film for attaching a surgical adjunct;

FIG. 27B shows an exemplary cartridge assembly with a post and an adhesive film for attaching a surgical adjunct;

FIG. 27C shows an exemplary cartridge assembly with a post, adhesive film, and surgical adjunct;

FIG. 27D is a top view of showing exemplary stakes, staple slots and a perforated film surrounding the staple slots;

FIG. 27E is a top view showing exemplary staples surrounding by films;

FIG. 28A is a flow chart showing an exemplary method for attaching a film to a polyurethane foam;

FIG. 28B is a flow chart showing an exemplary method of disposing a film on a polyurethane foam;

FIG. 28C is a flow chart showing an exemplary method for attaching a surgical adjunct to a cartridge using a film and posts; and

FIGS. 29A and 29B are flow charts showing exemplary methods for attaching an adjunct to a surgical staple cartridge using a biocompatible adhesive.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

In addition, throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Similarly, as used herein, “substantially free” of something, or “substantially pure”, and like characterizations, can include both being “at least substantially free” of something, or “at least substantially pure”, and being “completely free” of something, or “completely pure.”

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. Suitable aliphatic groups include, but are not limited to, linear or branched, substituted or unsubstituted alkyl, alkenyl, alkynyl groups and hybrids thereof such as cycloalkyl, (cycloalkyl)alkyl, (cycloalkenyl)alkyl or (cycloalkyl)alkenyl.

The term “alkyl,” unless otherwise indicated, as used herein, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, wherein the one or more substituents are independently C₁-C₁₀ alkyl. In some embodiments, “cycloalkyl” (or “carbocycle”) refers to a monocyclic C₃-C₆ hydrocarbon that is completely saturated or that contains one or more units of unsaturation, but which is not aromatic, that has a single point of attachment to the rest of the molecule. A carbocycle can be, under certain circumstances, a bridged bicyclic or a fused ring such as, e.g., an ortho-fused carbocycle, a spirofused carbocycle, etc. Examples of “alkyl” groups include, but are not limited to, methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, and the like.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “alkoxy” means an alkyl radical attached through an oxygen linking atom, represented by —O-alkyl. For example, “(C₁-C₄)alkoxy” includes methoxy, ethoxy, propoxy, and butoxy.

The term “aryl,” used alone or as part of a larger moiety, refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of compounds described herein, “aryl” refers to an aromatic ring system which includes, but is not limited to, phenyl, naphthyl, anthracyl and the like, which may bear one or more substituents. It will be appreciated that an “aryl” group can comprise carbon and heteroatom ring members.

As described herein, compounds described herein may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

As used herein, “degradation” refers to a process of breaking down of a polymer chain for absorption as atoms, molecules, or ions into the surroundings (e.g., tissue, cells, fluid, and the like). Degradation includes breaking primary and secondary bonds via thermal degradation, photodegradation, catalytic or enzymatic degradation, oxidative degradation, ion degradation, and biodegradation.

The term “polymerizable compound” means a compound containing one or more polymerizable groups. The term encompasses, for instance, monomers, macromers, oligomers, prepolymers, cross-linkers, and the like.

As used herein, “polymerizable groups” are groups that can undergo chain growth polymerization, such as free radical and/or cationic polymerization, for example a carbon-carbon double bond which can polymerize when subjected to radical polymerization initiation conditions. Non-limiting examples of free radical polymerizable groups include (meth)acrylates, styrenes, vinyl ethers, (meth)acrylamides, N-vinyllactams, N-vinylamides, O-vinylcarbamates, O-vinylcarbonates, and other vinyl groups. Preferably, the free radical polymerizable groups comprise (meth)acrylate, (meth)acrylamide, N-vinyl lactam, N-vinylamide, polyester polyols, poloxamers, and styryl functional groups, and mixtures of any of the foregoing. Preferably, the free radical polymerizable groups include (meth)acrylates, (meth)acrylamides, polyester polyols, poloxamers, and mixtures thereof. The polymerizable group may be unsubstituted or substituted. For instance, the nitrogen atom in (meth)acrylamide may be bonded to a hydrogen, or the hydrogen may be replaced with alkyl or cycloalkyl (which themselves may be further substituted).

The term “polyurethane,” as used herein, refers to a polymeric reaction product of an isocyanate and a polyol, and is not limited to those polymers which include only urethane or polyurethane linkages. It is well understood by those of ordinary skill in the art of preparing polyurethanes that the polyurethane polymers may also include linkages such as allophanate, carbodiimide, and other linkages described herein in addition to urethane linkages.

The term “polydrug,” as used herein, refers to a single drug and/or a drug including multiple active pharmaceutical ingredients (APIs) in a single dosage form or implant.

The expressions “reaction system,” “reactive formulation,” “reaction product,” and “reactive mixture” are interchangeably used herein, and all refer to a combination of reactive compounds used to make the bioabsorbable material according to the disclosure.

The term “room temperature” refers to temperatures of about 20° C., this means referring to temperatures in the range 18° C. to 25° C. Such temperatures will include 18° C., 19° C., 20° C., 21° C., 22° C., 23° C., 24° C. and 25° C.

Unless otherwise expressed, the “weight percentage” (indicated as % wt. or wt. %) of a component in a composition refers to the weight of the component over the total weight of the composition in which it is present and is expressed as percentage.

“Glass transition temperature” and “T_{g}” as referred to herein refers to the temperature at which a reversible transition from a hard glass condition into a rubber-elastic condition occurs.

The terms “NCO value” or “isocyanate value” as referred to herein is the weight percentage of reactive isocyanate (NCO) groups in an isocyanate, modified isocyanate, or isocyanate prepolymer compound.

Surgical stapling assemblies and methods for manufacturing and using the same are provided. In general, a surgical stapling assembly can include a staple cartridge having staples disposed therein and an adjunct configured to be releasably retained on the staple cartridge. As discussed herein, the various adjuncts provided can be configured to compensate for variations in tissue properties, such as variations in tissue thickness, and/or to promote tissue ingrowth when the adjuncts are stapled to tissue.

An exemplary stapling assembly can include a variety of features to facilitate application of a surgical staple, as described herein and illustrated in the drawings. However, a person skilled in the art will appreciate that the stapling assembly can include only some of these features and/or it can include a variety of other features known in the art. The stapling assemblies described herein are merely intended to represent certain exemplary examples. Moreover, while the adjuncts are described in connection with surgical staple cartridge assemblies, the adjuncts can be used in connection with staple reloads that are not cartridge based or any type of surgical instrument.

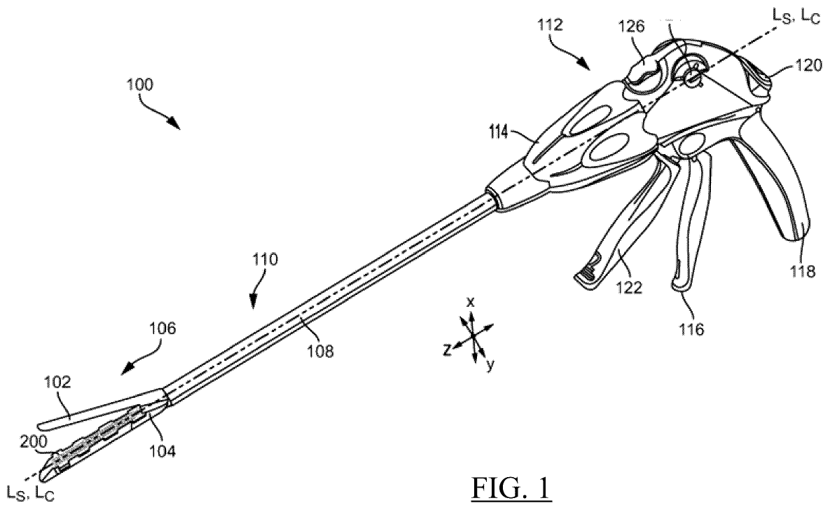

FIG. 1 illustrates an exemplary surgical stapling and severing device 100 suitable for use with an implantable adjunct. The illustrated surgical stapling and severing device 100 includes end effector 106 having an anvil 102 that is pivotably coupled to an elongate channel 104. As a result, the staple applying assembly 106 can move between an open position, as shown in FIG. 1, and a closed position in which the anvil 102 is positioned adjacent to the elongate channel 104 to engage tissue therebetween. The end effector 106 can be attached at its proximal end to an elongate shaft 108 forming an implement portion 110. When the end effector 106 is closed, or at least substantially closed, (e.g., the anvil 102 moves from the open position in FIG. 1 toward the elongate channel) the implement portion 110 can present a sufficiently small cross-section suitable for inserting the end effector 106 through a trocar. While the device 100 is configured to staple and sever tissue, surgical devices configured to staple but not sever tissue are also contemplated herein.

In various instances, the end effector 106 can be manipulated by a handle 112 connected to the elongate shaft 108. The handle 112 can include user controls such as a rotation knob 114 that rotates the elongate shaft 108 and the end effector 106 about a longitudinal axis (Ls) of the elongate shaft 108 and an articulation control 115 that can articulate the end effector 106 about an articulate axis (TA) that is substantially transverse to the longitudinal axis (Ls) of the elongate shaft 108. Further controls include a closure trigger 116 which can pivot relative to a pistol grip 118 to close the end effector 106. A closure release button 120 can be outwardly presented on the handle 112 when the closure trigger 116 is clamped such that the closure release button 120 can be depressed to unclamp the closure trigger 116 and open the end effector 106, for example. Handle 112 may also take the form of an interface for connection to a surgical robot.

In some examples, a firing trigger 122, which can pivot relative to the closure trigger 116, can cause the end effector 106 to simultaneously sever and staple tissue clamped therein. The firing trigger 122 may be a powered, require force from a user to engage, or some combination thereof. A manual firing release lever 126 can allow the firing system to be retracted before full firing travel has been completed, if desired, and, in addition, the firing release lever 126 can allow a surgeon, or other clinician, to retract the firing system in the event that the firing system binds and/or fails.

Additional details on the surgical stapling and severing device 100 and other surgical stapling and severing devices suitable for use with the present disclosure are described, for example, in U.S. Pat. No. 9,332,984 and in U.S. Patent Publication No. 2009/0090763, the disclosures of which are incorporated herein by reference in their entireties. Further, the surgical stapling and severing device need not include a handle, but instead can have a housing that is configured to couple to a surgical robot, for example, as described in U.S. Patent Publication No. 2019/0059889, the disclosure of which is incorporated herein by reference in its entirety.

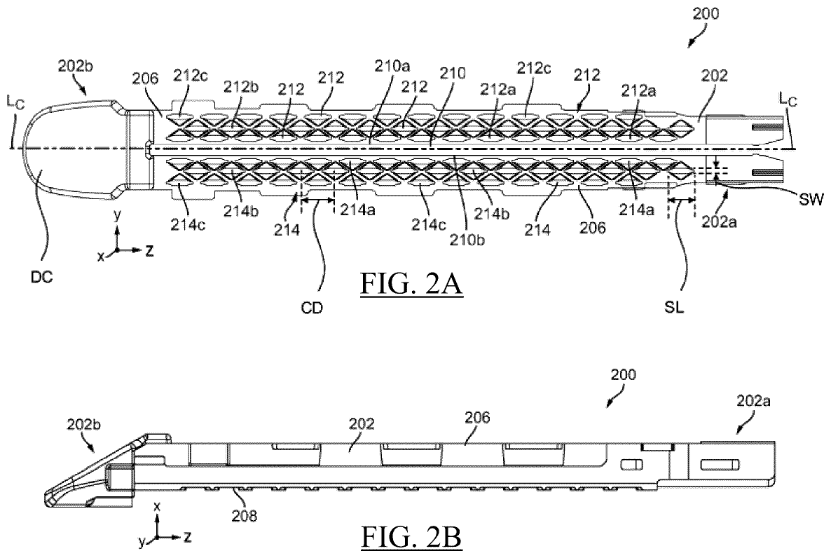

As further shown in FIG. 1, a staple cartridge 200 can be utilized with the instrument 100. In use, the staple cartridge 200 is placed within and coupled to the elongate channel 104. While the staple cartridge 200 can have a variety of configurations, in this illustrated example, the staple cartridge 200, which is shown in more detail in FIGS. 2A-2B, has a proximal end 202a and a distal end 202b with a cartridge longitudinal axis (LC) extending therebetween. As a result, when the staple cartridge 200 is inserted into the elongate channel 104 (FIG. 1), the longitudinal axis (LC) is substantially or approximately parallel with the longitudinal axis (LS) of the elongate shaft 108. Further, the staple cartridge 200 includes a longitudinal slot 210 defined by two opposing walls 210a, 210b and configured to receive at least a portion of a firing member of a firing assembly, like firing assembly 400 in FIG. 4, as discussed further below. As shown, the longitudinal slot 210 extends from the proximal end 202a toward the distal end 202b of the staple cartridge 200. It is also contemplated herein that in other examples, the longitudinal slot 210 can be omitted.

The illustrated staple cartridge 200 includes staple cavities 212, 214 defined therein, in which each staple cavity 212, 214 is configured to removably house at least a portion of a staple (not shown). The number, shape, and position of the staple cavities can vary and can depend at least on the size and shape (e.g., mouth-like shape) of the staples to be removably disposed therein. In this illustrated example, the staple cavities are arranged in two sets of three longitudinal rows, in which the first set of staple cavities 212 is positioned on a first side of the longitudinal slot 210 and the second set of staple cavities 214 is positioned on a second side of the longitudinal slot 210. On each side of the longitudinal slot 210, and thus for each set of rows, a first longitudinal row of staple cavities 212a, 214a extends alongside the longitudinal slot 210, a second row of staple cavities 212b, 214b extends alongside the first row of staple cavities 212a, 214a, and a third row of staple cavities 212c, 214c extends alongside the second row of staple cavities 212b, 214b. Each row may be approximately parallel and the staple cavities that make up the rows may be approximately parallel in orientation with the longitudinal slot 210. As shown in FIGS. 2A, each staple cavity 212, 214 may include a maximum length SL of about 0.122 inches to about 0.124 inches and a maximum width SW of about 0.023 inches to about 0.027 inches. In addition, at least the centers of two adjacent cavities 212, 214 are spaced apart by about 0.158 inches.

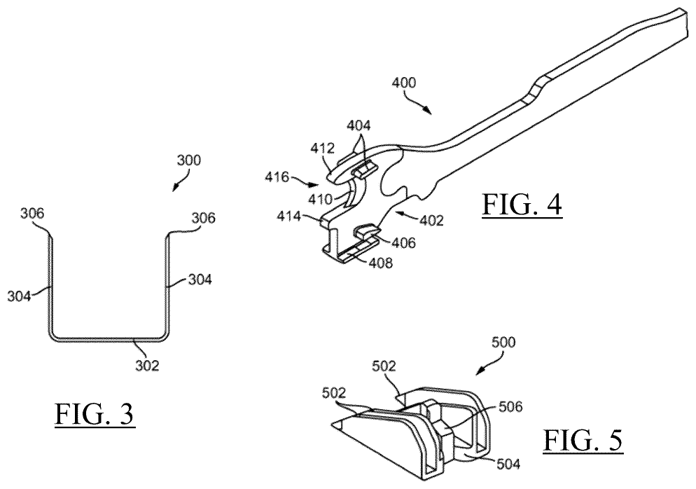

The staples releasably stored in the staple cavities 212, 214 can have a variety of configurations. An exemplary staple 300 that can be releasably stored in each of the staple cavities 212, 214 is illustrated in FIG. 3 in its unfired (pre-deployed, unformed) configuration. The illustrated staple 300 includes a crown (base) 302 and two legs 304 extending from each end of the crown 302. In this example, the crown 302 extends in a linear direction and the staple legs 304 have the same unformed height. Further, prior to the staples 300 being deployed, the staple crowns 302 can be supported by staple drivers that are positioned within the staple cartridge 200 and, concurrently, the staple legs 304 can be at least partially contained within the staple cavities 212, 214. Further, the staple legs 304 can extend beyond a top surface, like top surface 206, of the staple cartridge 200 when the staples 300 are in their unfired positions. In certain instances, as shown in FIG. 3, the tips 306 of the staple legs 304 can be pointed and sharp which can incise and penetrate tissue.

In use, staples 300 can be deformed from an unfired position into a fired position such that the staple legs 304 move through the staple cavities 212, 214, penetrate tissue positioned between the anvil 102 and the staple cartridge 200, and contact the anvil 102. As the staple legs 304 are deformed against the anvil 102, the legs 304 of each staple 300 can capture a portion of the tissue within each staple 300 and apply a compressive force to the tissue. Further, the legs 304 of each staple 300 can be deformed downwardly toward the crown 302 of the staple 300 to form a staple entrapment area in which the tissue can be captured therein. In various instances, the staple entrapment area can be defined between the inner surfaces of the deformed legs and the inner surface of the crown of the staple. The size of the entrapment area for a staple can depend on several factors such as the length of the legs, the diameter of the legs, the width of the crown, and/or the extent in which the legs are deformed, for example.

In some examples, all of the staples disposed within the staple cartridge 200 can have the same unfired (pre-deployed, unformed) configuration. In other examples, the staples can include at least two groups of staples each having a different unfired (pre-deployed, unformed) configuration, e.g., varying in height and/or shape, relative to one another, etc.

Referring back to FIGS. 2A-2B, the staple cartridge 200 extends from a top surface or deck surface 206 to a bottom surface 208, in which the top surface 206 is configured as a tissue-facing surface and the bottom surface 208 is configured as a channel-facing surface. As a result, when the staple cartridge 200 is inserted into the elongate channel 104, as shown in FIG. 1, the top surface 206 faces the anvil 102 and the bottom surface 208 (obstructed) faces the elongate channel 104.

With reference to FIGS. 4 and 5, a firing assembly such as, for example, firing assembly 400, can be utilized with a surgical stapling and severing device, like device 100 in FIG. 1. The firing assembly 400 can be configured to advance a wedge sled 500 having wedges 502 configured to deploy staples from the staple cartridge 200 into tissue captured between an anvil, like anvil 102 in FIG. 1, and a staple cartridge, like staple cartridge 200 in FIG. 1. Furthermore, an E-beam 402 at a distal portion of the firing assembly 400 may fire the staples from the staple cartridge. During firing, the E-beam 402 can also cause the anvil to pivot towards the staple cartridge, and thus move the end effector from the open position towards a closed position. The illustrated E-beam 402 includes a pair of top pins 404, a pair of middle pins 406, which may follow a portion 504 of the wedge sled 500, and a bottom pin or foot 408. The E-beam 402 can also include a sharp cutting edge 410 configured to sever the captured tissue as the firing assembly 400 is advanced distally, and thus towards the distal end of the staple cartridge. In addition, integrally formed and proximally projecting top guide 412 and middle guide 414 bracketing each vertical end of the cutting edge 410 may further define a tissue staging area 416 assisting in guiding tissue to the sharp cutting edge 410 prior to being severed. The middle guide 414 may also serve to engage and fire the staples within the staple cartridge by abutting a stepped central member 506 of the wedge sled 500 that effects staple formation by the end effector 106.

In use, the anvil 102 in FIG. 1 can be moved into a closed position by depressing the closure trigger in FIG. 1 to advance the E-beam 402 in FIG. 4. The anvil 102 can position tissue against at least the top surface 206 of the staple cartridge 200 in FIGS. 2A-2B. Once the anvil has been suitably positioned, the staples 300 in FIG. 3 disposed within the staple cartridge can be deployed.

To deploy staples from the staple cartridge, as discussed above, the sled 500 in FIG. 5 can be moved from the proximal end toward a distal end of the cartridge body, and thus, of the staple cartridge. As the firing assembly 400 in FIG. 4 is advanced, the sled can contact and lift staple drivers within the staple cartridge upwardly within the staple cavities 212, 214. In at least one example, the sled and the staple drivers can each include one or more ramps, or inclined surfaces, which can co-operate to move the staple drivers upwardly from their unfired positions. As the staple drivers are lifted upwardly within their respective staple cavities, the staples are advanced upwardly such that the staples emerge from their staple cavities and penetrate into tissue. In various instances, the sled can move several staples upwardly at the same time as part of a firing sequence.

As indicated above, the stapling device can be used in combination with a compressible adjunct. A person skilled in the art will appreciate that, while adjuncts are shown and described below, the adjuncts disclosed herein can be used with other surgical instruments and need not be coupled to a staple cartridge as described. Further, a person skilled in the art will also appreciate that the staple cartridges need not be replaceable.

As discussed above, with some surgical staplers, a surgeon is often required to select the appropriate staples having the appropriate staple height for tissue to be stapled. For example, a surgeon will utilize tall staples for use with thick tissue and short staples for use with thin tissue. In some instances, however, the tissue being stapled does not have a consistent thickness and thus, the staples cannot achieve the desired fired configuration for every section of the stapled tissue (e.g., thick and thin tissue sections). The inconsistent thickness of tissue can lead to undesirable leakage and/or tearing of tissue at the staple site when staples with the same or substantially greater height are used, particularly when the staple site is exposed to intra-pressures at the staple site and/or along the staple line.

Accordingly, various examples of adjuncts are provided that can be configured to compensate for varying thickness of tissue that is captured within fired (deployed) staples to avoid the need to take into account staple height when stapling tissue during surgery. That is, the adjuncts described herein can allow a set of staples with the same or similar heights to be used in stapling tissue of varying thickness (e.g., from thin to thick tissue) while also, in combination with the adjunct, providing adequate tissue compression within and between fired staples. Thus, the adjuncts described herein can maintain suitable compression against thin or thick tissue stapled thereto to thereby minimize leakage and/or tearing of tissue at the staple sites. In addition, exemplary adjuncts described herein may be configured to be essentially fully absorbed in the body over a period of 100 to 300 days depending on implanted location and tissue health.

Alternatively, or in addition, the adjuncts can be configured to promote tissue ingrowth. In various instances, it is desirable to promote the ingrowth of tissue into an implantable adjunct, to promote the healing of the treated tissue (e.g., stapled and/or incised tissue), and/or to accelerate the patient's recovery. More specifically, the ingrowth of tissue into an implantable adjunct may reduce the incidence, extent, and/or duration of inflammation at the surgical site. Tissue ingrowth into and/or around the implantable adjunct may, for example, manage the spread of infections at the surgical site. The ingrowth of blood vessels, especially white blood cells, for example, into and/or around the implantable adjunct may fight infections in and/or around the implantable adjunct and the adjacent tissue. Tissue ingrowth may also encourage the acceptance of foreign matter (e.g., the implantable adjunct and the staples) by the patient's body and may reduce the likelihood of the patient's body rejecting the foreign matter. Rejection of foreign matter may cause infection and/or inflammation at the surgical site.

In general, the adjuncts provided herein are designed and positioned atop a staple cartridge, like staple cartridge 200. When the staples are fired (deployed) from the cartridge, the staples penetrate through the adjunct and into tissue. As the legs of the staple are deformed against the anvil that is positioned opposite the staple cartridge, the deformed legs capture a portion of the adjunct and a portion of the tissue within each staple. That is, when the staples are fired into tissue, at least a portion of the adjunct becomes positioned between the tissue and the fired staple. While the adjuncts described herein can be configured to be attached to a staple cartridge, it is also contemplated herein that the adjuncts can be configured to mate with other instrument components, such as an anvil of a surgical stapler. A person of ordinary skill will appreciate that the adjuncts provided herein can be used with replaceable cartridges or staple reloads that are not cartridge based.

In various embodiments, the adjunct or bioabsorbable materials disclosed herein can be comprised of an absorbable polymer. In certain embodiments, an adjunct can be comprised of foam, film, fibrous woven, fibrous non-woven polyurethane, polyester, polycarbonate, polyorthoesters, polyanhydrides, polyesteramides, and/or polyoxaesters. In other embodiments, an adjunct can be a copolymer including, for example, PGA (polyglycolic acid), PGA/PCL (poly(glycolic acid-co-caprolactone)), PLA/PCL (poly(lactic acid-co-polycaprolactone)), PLLA/PCL, PGA/TMC (poly(glycolic acid-co-trimethylene carbonate)), PDS, PEPBO, and the like. In various embodiments, an adjunct can include an organic material such as, for example, carboxymethyl cellulose, sodium alginate, hyaluronic acid, and/or oxidized regenerated cellulose. In various embodiments, an adjunct has a durometer in the 3-7 Shore A (30-50 Shore 00) ranges with a maximum stiffness of 15 Shore A (65 Shore 00). In certain embodiments, an adjunct can undergo 40% compression under 3 lbf load, 60% compression under 6 lbf load, and/or 80% compression under 20 lbf load, for example. In certain embodiments, one or more gasses, such as air, nitrogen, carbon dioxide, and/or oxygen, for example, can be bubbled through and/or contained within the adjunct.

### Methods of Stapling Tissue

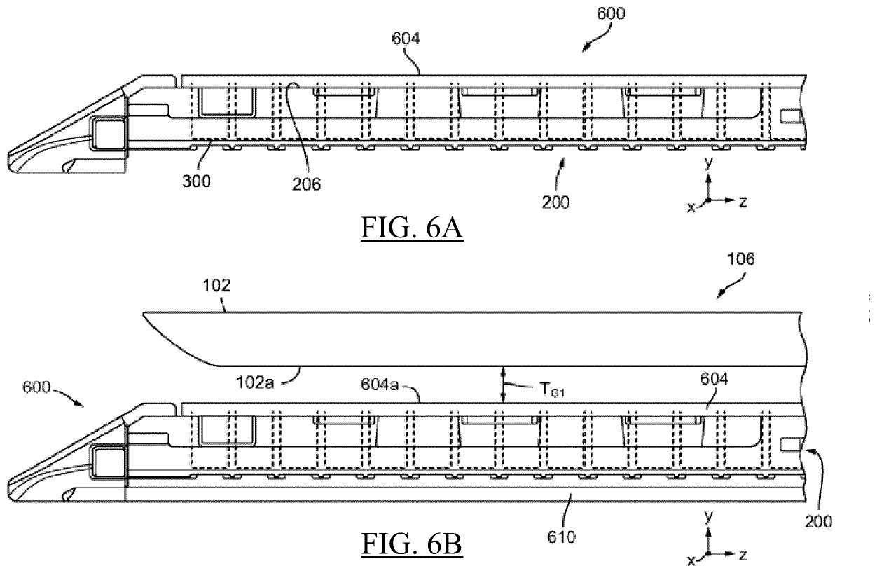

FIGS. 6A-6B illustrate an exemplary example of a stapling assembly 600 that includes a staple cartridge 200 and an adjunct 604. For sake of simplicity, the adjunct 604 is generally illustrated in FIGS. 6A-6B, and various configurations of the adjunct are described in more detail below. As shown, the adjunct 604 is positioned against the staple cartridge 200. While partially obstructed in FIGS. 6A-6B, the staple cartridge 200 includes staples 300, that are configured to be deployed into tissue. The staples 300 can have any suitable unformed (pre-deployed) height.

In the illustrated example, the adjunct 604 can be mated to at least a portion of the top surface or deck surface 206 of the staple cartridge 602. In some examples, the top surface 206 of the staple cartridge 200 can include one or more surface features which can be configured to engage the adjunct 604 to avoid undesirable movements of the adjunct 604 relative to the staple cartridge 200 and/or to prevent premature release of the adjunct 604 from the staple cartridge 200. Exemplary surface features are described further below and in U.S. Pat. No. 10,052,104, which is incorporated by reference herein in its entirety.

FIG. 6B shows the stapling assembly 600 placed within and coupled to the elongate channel 610 of surgical end effector 106. The anvil 102 is pivotally coupled to the elongate channel 610 and is thus moveable between open and closed positions relative to the elongate channel 610, and thus the staple cartridge 200. The anvil 102 is shown in a closed position in FIG. 6B and illustrates a tissue gap T_{G1} created between the staple cartridge 602 and the anvil 102. More specifically, the tissue gap T_{G1} is defined by the distance between the tissue-compression surface 102a of the anvil 102 (e.g., the tissue-engaging surface between staple forming pockets in the anvil) and the tissue-contacting surface 604a of the adjunct 604. In this illustrated example, both the tissue-compression surface 102a of the anvil 102 and the tissue-contacting surface 604a of the adjunct 604 is planar, or substantially planar (e.g., planar within manufacturing tolerances). As a result, when the anvil 102 is in a closed position, as shown in FIG. 6B, the tissue gap T_{G1} is generally uniform (e.g., nominally identical within manufacturing tolerances) when no tissue is disposed therein. In other words, the tissue gap T_{G1} is generally constant (e.g., constant within manufacturing tolerances) across the end effector 106 (e.g., in the y-direction). In other examples, the tissue-compression surface of the anvil can include a stepped surface having longitudinal steps between adjacent longitudinal portions, and thus create a stepped profile (e.g., in the y-direction). In such examples, the tissue gap T_{G1} can be varied.

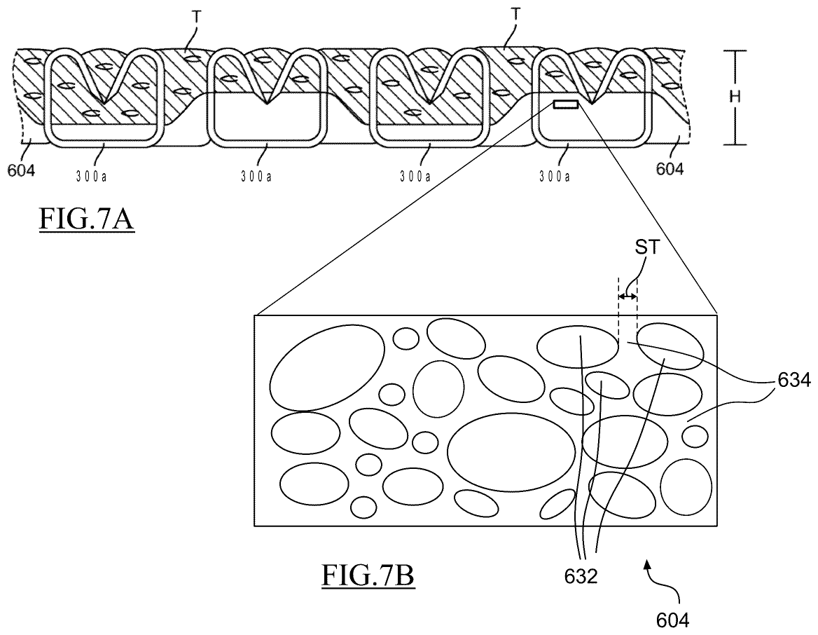

The adjunct 604 is compressible to permit the adjunct to compress to varying heights to thereby compensate for different tissue thickness that are captured within a deployed staple. The adjunct 604 has an uncompressed (undeformed), or pre-deployed, height and is configured to deform to one of a plurality of compressed (deformed), or deployed, heights. For example, the adjunct 604 can have an uncompressed height which is greater than the fired height of the staples 300 disposed within the staple cartridge 200 (e.g., the height (H) of the fired staple 300a in FIG. 7A). That is, the adjunct 604 can have an undeformed state in which a maximum height of the adjunct 604 is greater than a maximum height of a fired staple (e.g., a staple that is in a formed configuration).

In use, once the surgical stapling and severing device, like device 100 in FIG. 1, is directed to the surgical site, tissue is positioned between the anvil 102 and the stapling assembly 600 such that the anvil 102 is positioned adjacent to a first side of the tissue and the stapling assembly 600 is positioned adjacent to a second side of the tissue (e.g., the tissue can be positioned against the tissue-contacting surface 604a of the adjunct 604). Once tissue is positioned between the anvil 102 and the stapling assembly 600, the surgical stapler can be actuated, e.g., as discussed above, to thereby clamp the tissue between the anvil 102 and the stapling assembly 600 (e.g., between the tissue-compression surface 102a of the anvil 102 and the tissue-contacting surface 604a of the adjunct 604) and to deploy staples from the cartridge through the adjunct and into the tissue to staple and attach the adjunct to the tissue.

As shown in FIG. 7A, when the staples 300 are fired, tissue (T) and a portion of the adjunct 604 are captured by the fired (formed) staples 300a. The fired staples 300a each define the entrapment area therein, as discussed above, for accommodating the captured adjunct 604 and tissue (T). The entrapment area defined by a fired staple 300a is limited, at least in part, by a height (H) of the fired staple 300a.

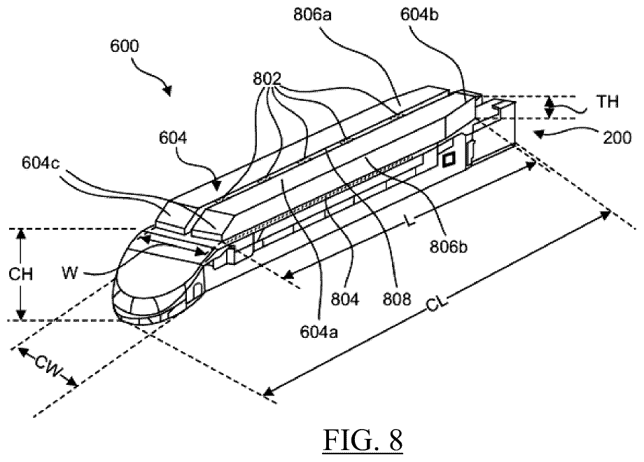

Referring to FIG. 7B, the adjunct 604 may have pores 632 with a median pore size of about 0.025 mm³ to about 0.300 mm³, such as about 0.022 mm³. In some examples, the adjunct 604 may have one or more struts 634 between the pore 632 that provide support and strength to the adjunct 604. In particular, the adjunct 604 may include a plurality of struts 634, having a median strut thickness ST of about 0.025 mm to about 0.300 mm, such as about 0.08 mm. FIG. 8 illustrates a perspective view of a staple cartridge assembly 600 with an adjunct 604 and a staple cartridge 200. The adjunct 604 has a tissue contacting surface 604a, a proximal end 604c, and a distal end 604b. The adjunct 604 may include a slot/slit 808 separating or partially separating two parallel portions of the adjunct 604. In one example, adjunct 604 may include a slot 808 separating two parallel portions of the adjunct 604, while in another example, adjunct 604 may include a slit 808 separating two parallel portions of the adjunct 604 and also one or more bridges (e.g., five bridges) 802 connecting the two parallel portions of the adjunct 604. At least one bridge has a length in the longitudinal direction of about 0.035 inches to about 0.046 inches. The adjunct 604 has a length L of about 40 mm to about 80 mm, such as about 60 mm to about 65 mm, about 66.04 mm to about 66.3 mm, about 45 mm to about 55 mm, or about 51.12 mm to about 51.38 mm. The adjunct 604 has a width W of about 8 mm to about 12 mm, such as about 9.75 mm to about 10.25 mm or about 10.025 mm to about 10.035 mm. The adjunct 604 may also have a thickness or height TH of about 2.5 mm to about 3.5 mm, such as about 2.85 mm to about 3.15 mm or about 2.95 mm to about 3.05 mm.

The cartridge 200 has a height CH of about 6.3 mm to about 8.1 mm, a width CW of about 8.9 mm to about 14 mm, and a length CL of about 80 mm to about 90 mm such as about 86.7 mm.

The staple cartridge 200 may include one or more raised ledges 804 along one or more sides of the adjunct 604 to help align the adjunct 604 on the deck of the staple cartridge 200. Although not shown in FIG. 8, the staple cartridge 200 may also include an adhesive or buttress adhesive material to attach the adjunct 604. The adjunct 604 may be attached to the cartridge 200 with about 100 mg to about 120 mg of the adhesive or buttress adhesive material.

### Adjusting Mechanical Properties Through Stimuli-Responsive Materials

The strength required to retain staples, sutures, screws, and the like, may conflict with the requirements for endoscopic deployment. As described above, the end effector 106 (shown in FIG. 1) comprising the cartridge 200 and adjunct 604 is closed or substantially closed for inserting through a trocar to the delivery site. As the implant strength increases (for maintaining staples and the hemostatic seal), there is an increased force required to compress the implant for insertion through the trocar. Therefore, the surgical adjunct 604 needs to have adjustable mechanical properties such that the material is sufficiently compressible during delivery through the trocar but increases strength at the delivery site to retain staples, sutures, and screws and maintain a hemostatic seal during the recovery period.

As described herein, the surgical adjunct 604 can be tuned for the particular purpose before, during, and after surgical procedures and have one or more of the functional groups described below that modulate the mechanical properties. In particular, the adjunct 604 described has one or more stimuli-responsive functional groups that respond to certain stimulus either ex vivo or in vivo, such that the compressibility of the adjunct 604 can be modulated during delivery and after firing.

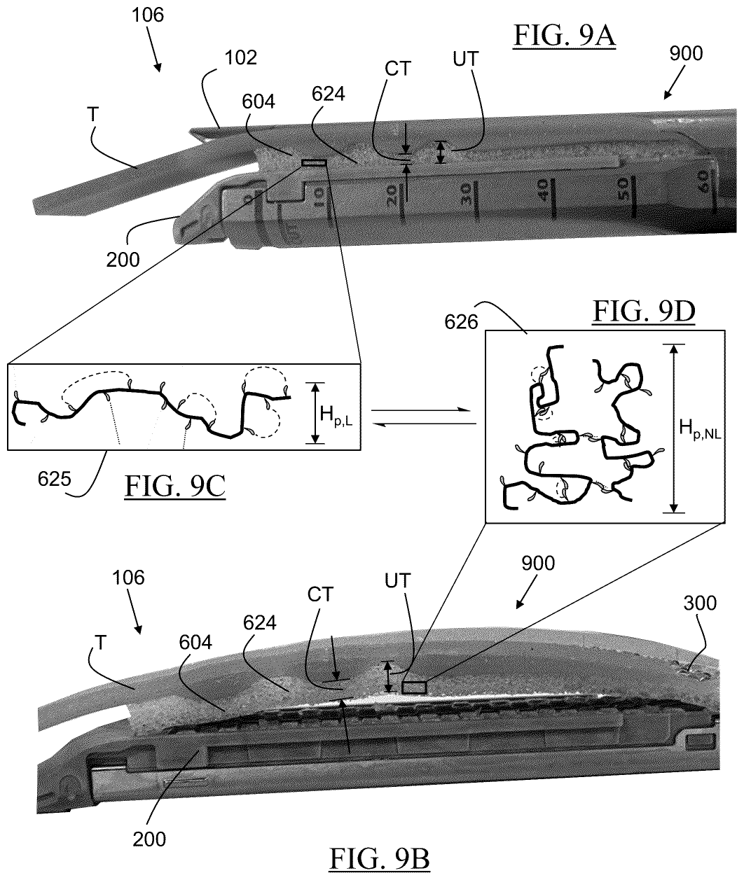

FIG. 9A is a side view of the end effector 106 in a delivery configuration, where the adjunct 604 is compressible between the anvil 102 and the cartridge 200. As illustrated, the end effector 106 can include a staple cartridge 200 and an adjunct 604 that is bioabsorbable can include a shape-memory polymer 624 that is compressible in a delivery configuration. In certain embodiments, the adjunct 604 is releasably retained on the cartridge 200. As illustrated in FIG. 9A, when the end effector 106 closes around tissue T, the adjunct 604 can compress from a thickness of the uncompressed adjunct UT to a thickness of compressed adjunct CT in response to a variation in tissue thickness.

FIG. 9B is a side view of the end effector 106 after firing of staples 300, where the adjunct 604 is no longer compressed between the anvil 102 and the cartridge 200. As shown, the adjunct 604 is released from the cartridge 200 after firing. The adjunct 604 is shown to maintain the thickness of the compressed adjunct CT and the thickness of uncompressed adjunct UT from pre-firing in FIG. 9A.

In some embodiments, the adjunct 604 can undergo a change in mechanical properties before firing of staples such that the uncompressed portion CT and compressed portion CT will be maintained after firing and releasing adjunct 604 and tissue T. In other embodiments, the adjunct 604 can undergo a change in mechanical properties after firing and within a predetermined time period. In such an example, the uncompressed portion CT and compressed portion CT can either expand or contract in response to the staple height and the tissue thickness as the tissue heals. As would be appreciated by those of skill in the art, a change in mechanical properties of the adjunct 604 can be advantageous to ensure a hemostatic seal during changes in tissue inflammatory responses and throughout the healing process.

To control mechanical properties of the bioabsorbable material, the adjunct 604 includes shape-memory polymer 624 that can swell via reversible transformations from linear polymer systems 625 to non-linear polymer systems 626, as shown in FIGS. 9C and 9D. Stimuli-responsive functional groups can allow for transitions from linear polymer systems to complex architectures such as star, cyclic, or hyperbranched systems. The transition between linear polymers 625 to non-linear polymers 626 can include bonding among adjacent functional groups along the same polymer strand, as shown by the dashed lines in FIGS. 9C and 9D. Alternatively, or in addition thereto, the bonding can be crosslinking among functional groups along adjacent polymer strands, as shown by the dotted lines between the primary strand of FIG. 9C and a secondary strand in FIG. 9D (secondary polymer strand is not illustrated in FIG. 9C for clarity). The shape-memory polymer may include a backbone including one or more polymers selected from polyurethane, polyether urethane, polyester urethane, polyester urea, polyester, polycarbonate, polyorthoester, polyanhydride, polyesteramide, polyphosphazenes, polyphosphoesters, polysaccharides, and/or polyoxaesters.

As would be understood by one of skill in the art, the polymer backbone may be further functionalized after formation of the backbone. For instance, a polyurethane backbone can be synthesized via a reaction between an isocyanate and a polyol. After formation of the polyurethane backbone, additional functional groups may be added. Alternatively, or in addition thereto, the polymer backbone precursors can be previously functionalized with one or more functional groups. As a non-limiting example of such a case, an isocyanate functionalized with one functional group may react with a polyol functionalized with a second functional group such that the polyurethane backbone is formed with pendant functional groups in the same step.

In certain embodiments, the shape-memory polymer 624 can be configured to adjust mechanical properties either through physical expansion (swelling) or a material phase change. The mechanical properties, such as the compression strength (compressing the polymer), tensile strength (stretching the polymer), flexural strength (bending of the polymer), torsional strength (twisting of the polymer), impact strength (under the effects of direct hammering or firing), tear resistance, ultimate elongation, and/or Young's modulus (ratio of stress to strain), can be configured to increase after releasing the shape-memory polymer 624 from the staple cartridge 200. The functional groups can undergo reversible bonding with adjacent functional groups via stimulation comprising at least one of heat, light, water, electrical, magnetic, electromagnetic, ultrasound, pH, and the like. As would be appreciated by one of skill in the relevant art, each functional group can be selective to transition upon only one type of stimulation or may be able to transition with a combination of stimuli.

The mechanical properties can be reversed when using dampening implants, for instance, when using an adjunct 604 that needs to be sutured in vivo. To prevent damage during implant manipulation and suturing procedures, the shape-memory polymer 624 can be configured to have increased mechanical properties, but can be specifically tailored to retain a rubbery state (to prevent tearing) upon exposure to biological conditions (increased temperatures, pH, and the like).

In some embodiments, the shape-memory polymer 624 can be programmed via temperature, such that the adjunct 604 is deformed by a maximum change in compression (eₘₐₓ) with a constant loading rate (e.g., 0.01 s⁻¹) at a programming temperature (T_{D}) and subsequently cooled to a loading temperature (T_{L}) while holding at the eₘₐₓ. Outside of potential relaxation (Δe), the strain is recovered once raised to the recovery temperature (T_{R}). In general, the adjunct 604 may be designed such that the glass transition temperature (T_{g}) is between T_{L} and T_{R}. In addition, the adjunct 604 may also be designed such that the transition occurs through effective plasticization or degradation such that the effective T_{g} depresses and allows for recovery with a liquid emersion step prior to deployment. Liquid emersion may be water or any in vivo fluids within the body at the delivery site.

In certain other embodiments, the shape-memory polymer 624 can be designed around light mediation such that bonds of the functional groups are created or broken upon exposure to differing wavelengths of light. Activation or deactivation of the bonds within these systems can be done with a transmittable wavelength through the tissue or within the endoscopic procedure.

Temperature-triggered architectural transformations can be achieved in the present system through the use of thermally labile functional groups. Example thermally labile functional groups include Diels-Alder adducts and/or azo groups. For instance, Diels-Alder linkages based on a furan-maleimide reaction can be formed at relatively low temperatures ranging from about room temperature (−25° C.) to about 60° C. The furan-maleimide reaction can be reversed at higher temperatures, such as equal to or greater than approximately 90° C. The retro-cycloaddition of the furan-maleimide reaction generates free furan and maleimide moieties. In general, for the Diels-Alder thermal transition, the increase in mechanical properties of the adjunct 604 can occur in the range of about 34° C. to about 60° C., such as in the range of about 34° C. to about 40° C.

In some embodiments, to achieve a change in mechanical properties under a Diels-Alder reaction, the backbone of the shape-memory polymer 624 can include a diene and a dienophile moiety. The diene and a dienophile moiety can be incorporated in the polymer's monomer backbone or as pendent groups.

In some examples, the shape-memory polymer 624 can include a suitable diene, such as substituted or unsubstituted alkene. In some embodiments, the suitable diene can include, without limitation, furans, thiophenes, or pyrroles. Without intending to be bound, some example dienes include, without limitation, substituted or unsubstituted 1,2-propadiene, isoprene, 1,3-butadiene, 2,4-octanedione, 1,5-cyclooctadiene, norbornadiene, 2-pyrone, dicyclopentadiene, 1H-pyrrole-2-carboxylic acid, 1H-pyrrole-3-carboxylic acid, 3,5-dimethyl-1H-pyrrole-2-carboxylic acid, 1,5-dimethyl-1H-pyrrole-2-carboxylic acid, 2,4,5-trimethyl-1H-pyrrole-3-carboxylic acid, 5-phenyl-1H-pyrrole-2-carboxylic acid, 2,4-dimethyl-1H-pyrrole-3-carboxylic acid, 2,5-dimethyl-1H-pyrrole-3-carboxylic acid, 3-methyl-1H-pyrrole-2-carboxylic acid, 5-(3,4-dimethylphenyl)-2-methyl-1H-pyrrole-3-carboxylic acid, 1-methyl-1H-pyrrole-2-carboxylic acid, 2-methyl-1H-pyrrole-3-carboxylic acid, furan-2-carboxylic acid, furan-3-carboxylic acid, 2-(furan-2-yl)acetic acid, 3-(5-methylfuran-2-yl)propanoic acid, 5-ethylfuran-2-carboxylic acid, 5-isobutyl-2-methylfuran-3-carboxylic acid, 4,5-dimethylfuran-2-carboxylic acid, thiophene-2-carboxylic acid, 4,5-dimethylthiophene-2-carboxylic acid, 3-methylthiophene-2-carboxylic acid, 5-methylthiophene-2-carboxylic acid, 5-phenylthiophene-2-carboxylic acid, 2-(thiophen-2-yl)acetic acid, thiophene-3-carboxylic acid, 2-(thiophen-3-yl)acetic acid, 5-ethylthiophene-2-carboxylic acid, and 5-methyl-4-phenylthiophene-3-carboxylic acid.

In some embodiments, the diene undergoes the Diels-Alder cycloaddition reaction with a suitable dienophile that can include either a substituted or unsubstituted alkene or alkyne. In some embodiments, a suitable dienophile can include, without limitation, substituted or unsubstituted maleimide, acrolein, methyl vinyl ketone, acrylic acid, methyl acrylate, acrylamide, acrylonitrile, methyl acrylate, dimethyl maleate, dimethyl fumarate, maleic anhydride, maleonitrile, butenolide, alpha-methylene gamma-butolactone, N-methylmaleimide, N-ethylmaleimide, dimethyl acetylene dicarboxylate, 6-maleimidohexanoic acid, 2-butenal, 2-Maleimidoacetic acid, 3-Maleimidopropionic acid, 3-Maleimidobenzoic acid, 3-(2,5-Dioxopyrrol-1-yl)hexanoic acid, 4-Maleimidobutyric acid, 4-Maleimidobenzoic acid, 4-(2,5-Dioxopyrrol-1-yl)hexanoic acid, 4-(2,5-dioxo-2,5-dihydro-pyrrol-1-yl)-benzoic acid, 5-Maleimidopentanoic acid, 6-Maleimidohexanoic acid, 6-(3-methyl-2,5-dioxopyrrol-1-yl)hexanoic acid, 6-(2,5-dioxopyrrol-1-yl)-2-methylhexanoic acid, 6-(2,5-Dioxopyrrol-1-yl)-4-methylhexanoic acid, 7-(2,5-Dioxo-2,5-dihydro-1H-pyrrol-1-yl)heptanoic acid, 9-(2,5-dioxopyrrol-1-yl)nonanoic acid, 10-(2,5-dioxopyrrol-1-yl)decanoic acid, 11-Maleimidoundecanoic acid, 13-(2,5-Dioxo-2,5-dihydro-1H-pyrrol-1-yl)tridecanoic acid, N-(Carboxyheptyl)maleimide, N-(4-Carboxy-3-hydroxyphenyl)maleimide, and α-Maleimidyl-ω-Carboxyl Poly(ethylene glycol).

In some embodiments, to achieve a change in mechanical properties under a thermal trigger, the backbone of the shape-memory polymer 624 can include an azo moiety. The azo moiety can be incorporated in the polymer's monomer backbone or as pendent groups. In general, for an azo thermal transition, the increase in mechanical properties of the adjunct 604 can occur in the range of about 60° C. to about 110° C. Alternatively, or in addition thereto, the azo moiety can be cleaved under light stimulation when the azo compound includes a substituted or unsubstituted aryl or heteroaryl groups within the azo molecule. Photoresponsive behaviors of the azo moiety within the shape-memory polymer 624 can be selected based on the azo compound. For example, an azo moiety with a trans-cis isomerization may be responsive to different wavelengths. For illustrative purposes, an azo moiety may experience photoresponsiveness in the trans-isomer at a wavelength ranging from about 350 nm to about 370 nm, whereas the cis-isomer may experience photoresponsiveness at a wavelength ranging from about 430 nm to about 460 nm. As would be appreciated by one of skill in the art, a shape-memory polymer containing one or more azo moieties may be tuned for a specific wavelength and/or temperature trigger.

In some examples, the shape-memory polymer 624 can include a suitable monozo, disazo, trisazo, polyazo, or azoic moiety. The monoazo moieties can be schematically represented by the formula Z—N═N—W, where Z and W are substituted or unsubstituted aryl or heterocyclic groups. Diazo moieties contain two —N═N— groups and can be symmetric or asymmetric. Polyazo moieties are characterized by the repetition of the azo group from three or more times in the same molecule. Suitable azo moieties can include, without limitation, substituted or unsubstituted triazolinediones, poly(vinylcarbazole), 1,1′-Azobis(cyclohexanecarbonitrile) or ACHN, 4,4′-azobis(4-cyanovaleric acid), azobenzene, 4,4-dihydroxyazobenzene, p-azobenzenearsonate, 2,2′-azobis(2-amidinopropane) dihydrochloride, 4,4′-azobis(4-cyanopentanoic acid), azobisisobutyronitrile, azodicarbonamide, azoxy compounds, para-azoxyanisole, azoxybenzene, balsalazide, 3-Hydroxy-4-[(2-hydroxy-5-methylphenyl)azo]-1-naphthalenesulfonic acid (“calmagite”), diethyl azodicarboxylate, diimide, diisopropyl azodicarboxylate, 4,4′-dinitro-3,3′-diazenofuroxan, 1,3-diphenyltriazene, disodium 4,4′-dinitrostilbene-2,2′-disulfonate, fazadinium bromide, 4H-1,2,4-triazole-3,4,5-triamine with 5,5′-(1,2-diazenediyl)bis[2H-tetrazole] (“G2ZT”), glycoazodyes, methylazoxymethanol, methylazoxymethanol acetate, olsalazine (also known under trade name “Dipentum”), phenazopyridine, 3-phenylazoacetylacetone, 7,18-bis(4-phenyldiazenylphenyl)-7,18-diazaheptacyclo[14.6.2.22,5.03,12.04,9.013,23.020,24]hexacosa-1(23),2,4,9,11,13,15,20 (24),21,25-decaene-6,8,17,19-tetrone (“pigment red 178”), potassium azodicarboxylate, 4-[(E)-{4-formyl-5-hydroxy-6-methyl-3-[(phosphonooxy)methyl]pyridin-2-yl}diazenyl] benzene-1,3-disulfonic acid (“PPADS”), 6-methyl-2-(phenylazo)-3-pyridinol (“SIB-1757”), 4-phenyldiazenylphenol (“solvent yellow 7”), sulfasalazine, tetramethylazodicarboxamide, disodium 3-hydroxy-4-[(2-arsonophenyl)diazenyl]naphthalene-2,7-disulfonate (“thorin”), and 1,3,5-tri(p-glycosyloxyphenyl azo)-2,4,6-trihydroxybenzene (“Yariv reagent”).

In some embodiments, to achieve a change in mechanical properties under photo-stimulation, the backbone of the shape-memory polymer 624 can include a styrylpyrene moiety. The styrylpyrene moiety can be incorporated in the polymer's monomer backbone or as pendent groups. Photoresponsive behaviors of the styrylpyrene moiety within the shape-memory polymer 624 can be selected to tune the wavelength of light required to initiate the change in mechanical properties. For instance, the shape-memory polymer 624 having a styrylpyrene moiety can be configured to undergo reversible transition of mechanical properties upon exposure to a wavelength of light ranging from about 310 nm to about 450 nm.

In some examples, the shape-memory polymer 624 can include a suitable styrylpyrene moiety. Suitable styrylpyrene moieties can include, without limitation, substituted or unsubstituted 1-styrylpyrene, phenanthracene, 3,4-benzopyrene, 1,2:5,6-dibenzanthracene, 1,2-benzanthracene, 7,12-dimethylbenzanthracene (“DMBA”), 1-[(1R,2S,4R)-5,6-dimethyl-2-bicyclo[2.2.1]heptanyl] pyrene, 1-(2-phenylethenyl)acenaphthylene, 2-(2-phenylethyl)dibenzofuran, 3-(2-phenylsulfanylethyl)-2-thia-3-azatricyclo[6.3.1.04,12]dodeca-1(11),4,6,8(12),9-pentaene, 1-(2-phenylethynyl)phenanthrene, 4-(2-phenylethenyl)pyrene, 2-(pyridin-2-ylmethoxy)-1,10-phenanthroline, 1-(3-phenylpropyl)imidazo[2,1-b][1,3]benzothiazole, trimethyl-(4-pyren-1-ylphenyl)silane, 1-methyl-2-methylidene-6-(1-methylnaphthalen-2-yl)-3-prop-2-enyl-3,3a-dihydro-1H-acenaphthylene, and the like.

In some embodiments, to achieve a change in mechanical properties under photo-stimulation, the backbone of the shape-memory polymer 624 can include an ortho-nitrobenzyl moiety. The ortho-nitrobenzyl moiety can be incorporated in the polymer's monomer backbone or as pendent groups. In general, the shape-memory polymer 624 having an ortho-nitrobenzyl moiety is configured to reversibly transition upon exposure to a wavelength of light ranging from about 310 nm to about 440 nm.

In some examples, the shape-memory polymer 624 can include a suitable ortho-nitrobenzyl moiety. Suitable ortho-nitrobenzyl moieties can include, without limitation, substituted or unsubstituted [4-[(2-nitrophenyl)methyl]phenyl]methanediol, 1-(4,5-dimethoxy-2-nitro-phenyl)-but-3-ene-1-ol, 4-((1-(4,5-dimethoxy-2-nitrophenyl)but-3-en-1-yl) oxy)-4-oxobutanoic acid, 3-((2-acryloyloxymethyl-2-hydroxymethyl)propionyloxy)methyl-2-nitrobenzyl, 4-cyano-4-(phenylcarbonothioylthio) pentanoate (“ANCP”), 2-nitrobenzyl cyclohexylcarbamate, 2-[(2-nitrobenzyl)oxy]-1H-isoindole-1,3(2H)-dione, 2-[(2-Nitrophenyl)methylthio]-1,3-benzoxazole, 2-(2-Methyl-5-nitroimidazol-1-yl)ethyl thiophene-2-carboxylate, (E)-3-(2-chlorophenyl)-N-cyclopentyl-2-propenamide, (2E)-N-(5-chloropyridin-2-yl)-3-(2-methoxyphenyl)acrylamide, N-[(2-nitrophenyl)methylideneamino] thiophene-2-carboxamide, N-cyclohexyl-3-(2-methoxyphenyl)prop-2-enamide, (2E)-N-cyclo heptyl-3-(2-nitrophenyl)prop-2-enamide, 2-(2-nitrophenyl)-N-(thiophen-2-ylmethyl)acetamide, N-(3,5-dichloro-4-methylpyridin-2-yl)-2-(1-oxidopyridin-1-ium-2-yl)sulfanylacetamide, 1-(2-Nitrophenyl)-3-phenyl-2-thiourea, N-bicyclo[2.2.1]hept-2-yl-2-(2-nitrophenyl)acetamide, 2-[2-(2-Chloroanilino)-2-oxoethyl]sulfanylbenzoic acid, N-(2,5-dimethylphenyl)-3-methyl-1-oxo-3,4-dihydro-1H-isochromene-3-carboxamide, [(2-nitrophenyl)amino]-N-(1,3,4-thiadiazol-2-yl) carboxamide, 2-cyano-N-(2-methylcyclohexyl)-3-(5-methylthiophen-2-yl)prop-2-enamide, (E)-2-cyano-N-(2-methylcyclohexyl)-3-(3-methylthiophen-2-yl)prop-2-enamide, (E)-1-(2-nitrophenyl)-N-phenylmethoxymethanimine, N-(2-nitrophenyl)-N′-pyridin-2-ylurea, 2-(2,4-dimethyl-6-nitrophenoxy)-N-(5-methyl-1,2-oxazol-3-yl)propanamide, 2-(2-Nitrophenoxy)-1-(2-phenylpyrrolidin-1-yl)ethanone, 4-Chloro-1-[2-(4-methoxyphenoxy)ethylsulfanyl]-2-nitrobenzene, 2-[2-Oxo-2-(thiophen-2-ylmethylamino)ethyl]sulfanylbenzoic acid, 1-(2-nitrophenyl)-N-phenylmethoxymethanimine, N-(2-chloropyridin-3-yl)-2-[(2-nitrothien-3-yl)thio]acetamide, 2-(2-Nitrothiophen-3-yl)sulfanyl-1-pyrrolidin-1-ylethanone, (2-nitrophenyl)methyl N-[1-(2-hydroxyethoxy)-5-methylhexan-3-yl]carbamate, and the like.

In some embodiments, to achieve a change in mechanical properties, the backbone of the shape-memory polymer 624 can be a coumarin moiety. The coumarin moiety can be incorporated in the polymer's monomer backbone or as pendent groups. Coumarin moieties undergo a reversible [2πs+2πs] cycloaddition reaction upon irradiation with specific wavelengths in the UV region, which is applied to impart intrinsic healability, shape-memory, and reversible properties into polymers. During photoirradiation, four different types of coumarin dimers are formed: anti head-to-head, anti head-to-tail, syn head-to-head, and syn head-to-tail.

In some examples, the shape-memory polymer 624 can include a suitable coumarin or coumarin-derivative moiety including dihydrofurano coumarins, furano coumarins, pyrano coumarins, phenyl coumarins, and bicoumarins. Suitable coumarin and/or coumarin-derivative moieties can include, without limitation, substituted or unsubstituted 2H-1-benzopyran-2-one (“coumarin”), 2-(Dimethylamino)ethyl methacrylate (DMAEMA) 7-(2-methacryloyloxyethoxy)-4-methylcoumarin (CMA), poly(DMAEMA-co-CMA). Dimethylaminoethyl acrylate (DMAEA), 6-Iodo-2H-chromen-2-one, 4-Hydroxycoumarin, 3-Hydroxycoumarin, 6-Methoxycoumarin, 4-Trimethylsiloxycumarin, and the like. In some embodiments, the shape-memory polymer 624 may be functionalized with coumarinyl end groups such that a single polymer strand can undergo a photodimerization.

In some embodiments, to achieve a change in mechanical properties, the backbone of the shape-memory polymer 624 can be an anthracene moiety. The anthracene moiety can be incorporated in the polymer's monomer backbone or as pendent groups. In general, anthracene groups undergo [4+4] photo-dimerization when irradiated by UV light (λ>300 nm) and can be reversed to the original monomers via exposure to a higher energy UV light (λ<300 nm).

In some examples, the shape-memory polymer 624 can include a suitable anthracene or anthracene-derivative moiety. Suitable anthracene and/or anthracene-derivative moieties can include, without limitation, substituted or unsubstituted Benzo[a]pyrene, Phenothiazine, Anthranol, Dibenzothiophene 5-oxide, 1,4,5-Trimethylnaphthalene, 4-Methyldibenzothiophene, Pyrene, 10-Methylacridin-9(10H)-one, gamma-Fagarine, 9-Hydroxymethyl-10-methylanthracene, 2-Dodecylphenanthrene, Phenanthrene, 2-dodecyl-9,10-dihydro-, 2-Octyltriphenylene, 8a-Methyl-3,5-dimethylidene-3a,4,4a,6,7,8,9,9a-octahydrobenzo[f][1]benzo furan-2-one, Furanoeremophilane, 4-[(E)-2-(1-naphthyl)vinyl]biphenyl, 1,7-diazatricyclo [7.3.0.03,7]dodeca-3,5,9,11-tetraene-2,8-dione (“Pyrocoll”), (3R,4aR,8aR)-5,8a-dimethyl-3-prop-1-en-2-yl-2,3,4,4a,7,8-hexahydro-1H-naphthalene (“alpha-Selinene”), 3,4-dihydro-2H-pyrimido[1,2-b][1,2]benzothiazole, and the like. In some embodiments, the shape-memory polymer 624 may be functionalized with anthracene end groups such that a single polymer strand can undergo a photodimerization.

In some embodiments, to achieve a change in mechanical properties, the backbone of the shape-memory polymer 624 can include a disulfide moiety. The disulfide moiety is a functional group with the formula R—S—S—R′, where R and R′ are either the same or different groups. The disulfide moiety can be incorporated in the polymer's monomer backbone or as pendent groups.

In general, disulfide moieties can be redox-responsive. Redox-active disulfide bonds are reversible and responsive to changes in the redox potential of the surrounding environment, and the formation or reduction of these disulfide bonds serve to increase the mechanical properties of the shape-memory polymer 624 after firing and delivery. The disulfide moieties can be intramolecular (oxidoreductases, allosteric disulfides, etc.) or mixed disulfides between a cysteine residue and a small-molecule thiol resulting in glutathionylated and cysteinylated adducts.

In some examples, the shape-memory polymer 624 can include a suitable disulfide moiety including, without limitation, substituted or unsubstituted diamines with disulfide groups, thiols with disulfide groups, or inimers with disulfide groups. Suitable disulfide moieties can include, without limitation, substituted or unsubstituted thioredoxin disulfide, 2-(2′-bromoisobutyryloxy)ethyl-2″-methacryloyloxyethyl disulfide, and the like.

In some embodiments, to achieve a change in mechanical properties, the backbone of the shape-memory polymer 624 can be a diselenide moiety. The diselenide moiety can be incorporated in the polymer's monomer backbone or as pendent groups.

In general, diselenide moieties can be redox-responsive. Selenolate-diselenide equilibria are the same as their sulfur-containing counterparts, thiolate-diselenide equilibria, and involve the reversible formation of diselenides from selenolates through the two-electron oxidation of the selenolate group.

In some examples, the shape-memory polymer 624 can include a suitable diselenide moiety including, without limitation, substituted or unsubstituted selenols, diselenides, selenides, selenoxides, selenoketones, selenones, selenenic acids, or seleninic acids. Suitable diselenide moieties can include, without limitation, substituted or unsubstituted diselenocarbonate. selenocysteamine, selenocystine, selenocystine, glutathione, oxidized glutathione, and selenocystamine, and the like.

In some embodiments, to achieve a change in mechanical properties, the backbone of the shape-memory polymer 624 can be at least one of a diene and a dienophile moiety, a styrylpyrene moiety, an azo moiety, an ortho-nitrobenzyl moiety, a coumarin moiety, an anthracene moiety, a disulfide moiety, a diselenide moiety, or combinations thereof.

In general, these molecular changes via bond formation and breaking can be tailored to allow a macroscopic geometric change in the shape-memory polymer 624. The transformation of the shape-memory polymer 624 can be thermal, photo, redox, or mechano-responsive.

In some embodiments, the functional groups along the shape-memory polymer 624 are more highly concentrated along a portion of the adjunct 604 so as to form a gradient of compression strength along a portion of the porous body 634. In general, the adjunct 604 may have a compression strength of about 30 kPa to about 70 kPa, such as about 30 kPa to about 60 kPa (e.g., about 42 kPa), about 30 kPa to about 50 kPa, about 32.5 kPa to about 37.5 kPa. In some embodiments, the adjunct 604 may have a compression strength of about 15 to about 50 kPa in the second zone during delivery and a compression strength of about 30 kPa to about 70 kPa after delivery through the trocar, but before firing of staples. In order to test compression strengths, an adjunct 604 was placed in a humid warm environment at approximately 37° C., compressed to a first height, then a second height shorter than the first height, and then released back to the first height at which point the adjunct's compression strength was measured.

In any of the embodiments described herein, the adjunct 604 can be configured to reversible transition between an approximately linear polymer and an approximately non-linear polymer within approximately 0.01 seconds to approximately 15 minutes such that the swelling of the shape-memory polymer 624 occurs between the time of deployment through the trocar and firing of the staples, including various surgical procedure time delays. In some cases, the stimulation may cause the reversible transition to occur much faster, such that the shape-memory polymer 624 reaches substantial swelling and compression strength within seconds of being delivered through the trocar (e.g., within approximately 1 second, 2 seconds, 3 seconds, 4 seconds, 5 seconds, 10 seconds, 15 seconds, 20 seconds, 30 seconds, and any time in between). In other embodiments, the shape-memory polymer 624 may be configured to reach substantial swelling and compression strength within a delayed time period from exposure to stimulation (e.g., after approximately 30 seconds, after 45 seconds, after 1 minute, after 2 minutes, and any time in between).

In some embodiments, the adjunct 604 may have a lower compressive strength during delivery and the compressive strength or other mechanical properties only increase after exposure to stimulus after deployment. Alternatively, the adjunct 604 can be compressed to a small thickness having high compressive strength but better margins for delivery through a trocar.

In some examples, the adjunct 604 may have a tensile strength of about 30 kPa to about 90 kPa such as about 45 kPa to about 85 kPa or about 55 kPa to about 75 kPa during delivery. After delivery, the adjunct 604 may increase the tensile strength from about 30 to about 45 kPa, or from about 45 kPa to about 65 kPa, or from about 55 kPa to about 75 kPa after exposure to stimulus. In some examples, the adjunct 604 will have tensile strength of about 110 kPa to about 150 kPa during delivery, that can increase upon exposure to a stimulus to a range of about 140 kPa to about 220 kPa.

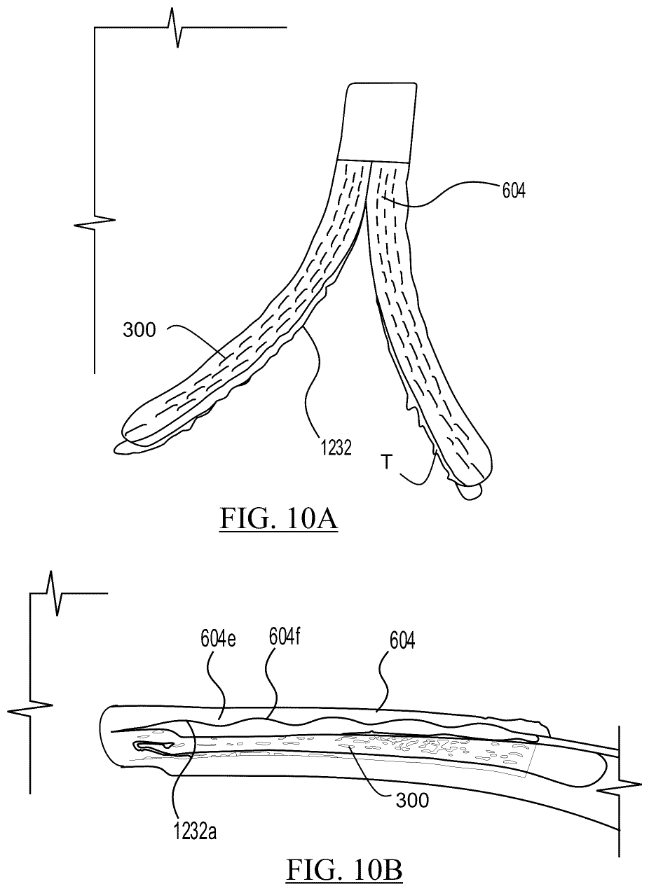

FIGS. 10A and 10B show top and side view of an adjunct 604 after firing staples 300 and cutting tissue T. Adjunct 604 may split in two post firing and the adhesive 1232 adhering the adjunct 604 to the cartridge 200 (see FIGS. 6A and 6B) may with the adjunct 604 form bumps 604e, 604f in the adjunct and corresponding bumps 1232a in the adhesive 1232. Where the adjunct 604 contacts with the tissue T, bumps 604e, 604f may form that correspond to the texture and thickness variation in the tissue. This means that adjunct 604 can adapt to different heights and compressions depending on the application.

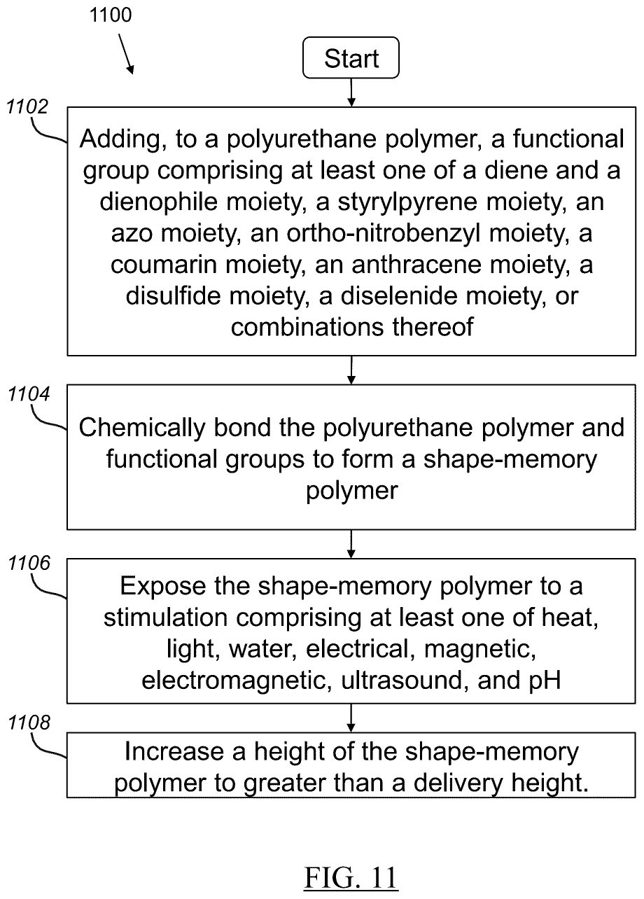

FIG. 11 is a flowchart of a method 1100 for forming a surgical adjunct 604, including a bioabsorbable material that itself includes a shape-memory polymer. The techniques for adjusting mechanical properties described herein may offer an added benefit of increasing adjunct strength and durability in combination with positive in vivo interactions (e.g., biocompatibility, wound healing, tissue integration, chemotherapy, anti-inflammatory, bone growth and integration, ligament and tendon repair, etc.) when the adjunct is delivered to the tissue site, as described herein. As such, the techniques described herein may allow the bioabsorbable material itself to aid in the healing process of the surrounding tissue. In addition, the embedding techniques described herein may offer an added benefit of preventing fibrous encapsulation of the foam cushion, and/or providing tunable release profiles for a variety of medical additives delivered to the tissue site.

Specifically with respect to FIG. 11, method 1100 used for forming a bioabsorbable material (e.g., a foam) having shape-memory polymer may include adding, to a polyurethane polymer, a functional group that can reversibly crosslink upon exposure to stimulation (step 1102). The functional group can be at least one of a diene and a dienophile moiety, a styrylpyrene moiety, an azo moiety, an ortho-nitrobenzyl moiety, a coumarin moiety, an anthracene moiety, a disulfide moiety, a diselenide moiety, or combinations thereof. Method 1100 can next include chemically bonding the polyurethane polymer and functional groups to form the shape-memory polymer (step 1104). In some examples, the bioabsorbable material may include a polyurethane, or alternatively, may include polyether urethane, polyester urethane, polyester urea, polyester, polycarbonate, polyorthoester, polyanhydride, polyesteramide, polyphosphazenes, polyphosphoesters, polysaccharides, and/or polyoxaester. Method 1100 also includes exposing the shape-memory polymer to a stimulation comprising at least one of heat, light, water, electrical, magnetic, electromagnetic, ultrasound, and pH (step 1106). After exposure to the stimulation, method 1100 includes increasing a height of the shape-memory polymer to greater than a delivery height (step 1108). Method 1100 can end after step 1108 or can optionally include adding a medical additive to the porous body. In such examples, the medical additives may include medicants to treat pain and/or promote wound healing, tissue growth, infection reduction, and the like.

### Tunable Absorption Pathways for Bioabsorbable Material

Balancing mechanical property depletion with backbone absorption of the adjunct after delivery may be improved with a dual phase degradation mechanism. As described above, the end effector 106 (shown in FIG. 1) comprising the cartridge 200 and adjunct 604 is closed or substantially closed for inserting through a trocar to the delivery site. Therefore, the surgical adjunct 604 needs to have mechanical properties such that the material is sufficiently compressible during delivery to be inserted through a trocar, but must have increased strength to retain staples, sutures, and screws at the delivery site. In addition, the surgical adjunct 604 needs to be bioabsorbable such that after delivery, the adjunct 604 remains at the target site until the tissue can sufficiently maintain a hemostatic seal and/or the tissue is sufficiently healed. From a mechanical standpoint, the adjunct 604 provides an ideal amount of compression that is both strong enough to create and maintain the hemostatic seal and is not over-compressing or too tough to permit firing of the staples upon delivery. A short-term absorption profile may be preferred to address hemostasis while a long-term absorption profile may address better tissue healing without leakages.

Returning to FIGS. 9A and 9B, an example staple cartridge assembly 900 includes an end effector 106 having a surgical adjunct 604 and a staple cartridge 200. The surgical adjunct 604 can be composed of a substantially monolithic structure. In addition, the adjunct 604 can be a bioabsorbable material that includes a first polymerizable compound that has a first degradation profile. The material choice of the first polymerizable compound can be a reaction product of a polyol and an isocyanate, such as polyurethane or a like polymer including, without limitation, polyether urethane, polyester urethane, polyester urea, polyester, polycarbonate, polyorthoester, polyanhydride, polyesteramide, polyphosphazenes, polyphosphoesters, polysaccharides, and/or polyoxaester. The polyurethane reaction product can have a degradation profile that is tailored for the target tissue site. The surgical adjunct 604 can be tuned the particular purpose before, during, and after surgical procedures and have one or more of the properties described below that modulate the mechanical properties. In particular, the adjunct 604 described has selective crosslinking density control in vivo and can also have a certain compressibility when attached to the cartridge outside of the body.

In some embodiments, the first polymerizable compound can be a reaction product of a polyol and an isocyanate, forming a polyurethane or polyurethane derivate. The polyurethane is configured to degrade according to a first degradation profile. An aliphatic polyurethane primarily undergoes oxidative and/or enzymatic mechanisms of absorption. To maintain the mechanical properties when firing the staples while tuning the absorption profile after minutes, hours, days, or weeks after delivery, a second polymerizable compound is added. The second polymerizable compound is configured to degrade according to a second degradation profile. In some examples, the second polymerizable compound can adjust an induction period for absorption, thereby delaying bulk absorption of the adjunct 604. In some instances, the physiological conditions at the delivery site can be used to accelerate absorption of at least one of the first polymerizable compound and/or the second polymerizable compound.

As described herein, the surgical adjunct 604 can be tuned for the particular purpose before, during, and after surgical procedures. The adjunct 604 includes a first polymerizable compound that has a first degradation profile and a second polymerizable compound with a second degradation profile different than the first. In particular, the adjunct 604 described can be a polymer with tunable absorption properties via an alternating copolymer, a random copolymer, a block copolymer, a multiblock copolymer architecture, a terpolymer, a graft copolymer (monomer backbone with pendant chains of a different monomer), a bulk homopolymer coated with a second homopolymer, and the like.

In some embodiments, the second polymerizable compound can be a polyester, a poloxamer, a polymethacrylate, a polyether, a polydioxanone, a polyanhydride, a hydroxypropyl methylcellulose acetate succinate, a cellulose acetate phthalate, a cellulose acetate trimellitate, a hydroxypropyl methylcellulose phthalate, a polyvinyl acetate phthalate, a poly(trimethylene carbonate), a poly(beta-thioether ester ketal), a polypropylene fumarate, a poly(ester urea), a poly(ester amide).

In some embodiments, the first polymerizable compound alone has a suitable degradation profile for healing tissue within approximately 6 weeks. In some embodiments, it is desirable to tune the degradation profile of the adjunct 604 for longer degradation profiles. In some embodiments, the bioabsorption rate of the adjunct 604 can be tuned by adding more or less of the second polymerizable compound to the first polymerizable compound. In general, the adjunct 604 can have approximately equal parts of the first polymerizable compound to second polymerizable compound. In certain other embodiments, the volumetric ratio of first polymerizable compound to second polymerizable compound can range from about 10:0.1 to about 0.1:10. For instance, when it is desirable to have a longer degradation profile, the volumetric ratio can be approximately 10:1, about 10:2, about 10:3, or about 10:4. As another non-limiting example, to tune the degradation profile to shorter periods of time, the volumetric ratio can be approximately 1:10, approximately 2:10, approximately 3:10, or approximately 4:10. As would be appreciated by one of skill in the relevant art, tuning the degradation profile of the adjunct as a whole can be done through adjusting the type, molecular weight, and quantity of first polymerizable compound to second polymerizable compound.

In some embodiments, the second polymerizable compound can be added to the polyurethane backbone such that the first and second polymerizable compounds together form a copolymer backbone. When the second polymerizable compound is added to the polyurethane backbone, the second polymerizable compound can be one of the polyol or the isocyanate. For instance, the second polymerizable compound can be a polyester polyol that when added to the first polymerizable compound, the polyester polyol can replace the polyol of the polyurethane reaction product to form a block copolymer or an alternating copolymer. Alternatively, or in addition thereto, the polyester polyol can be in addition to the polyol of the polyurethane reaction product such that more than one type of polyol monomer is introduced into the backbone of the polyurethane, such as in a block copolymer (A-B-A style) or a random copolymer.

The inclusion of an aliphatic polyester polyol as the second polymerizable compound, can add another degree of freedom in a polyurethane backbone to tune the absorption and mechanical properties of the adjunct 604. Above a certain molecular weight, mechanical toughening can occur through crystallization that effectively adds crosslinks to the polyurethane system and thus allow for lower covalent crosslinks overall. In general, reducing the number of covalent crosslinks can result in an accelerated degradation profile of the adjunct 604.

In addition to the change in crosslinking density, the addition of the second polymerizable compound such as polyester polyols, can increase the backbone susceptibility to hydrolysis mechanism of absorption. Under a hydrolysis mechanism pathway, the sensitivity to acidic conditions could be used to preferentially increase the absorption rate or degradation profile of the adjunct 604 in the presence of lower pH.

The typical polyester polyol is branched with a weight average molecular weight (Mw) of 2,000-10,000. Polyester polyols are typically made from mixtures of diols, triols, and dibasic acids or anhydrides. Polyether polyols are made by the reaction of epoxides with compounds having active hydrogen atom. Polyester polyols are made by the polycondensation reaction of multifunctional carboxylic acids and polyhydroxyl compounds. Example polyester polyol monomers include, without limitation, poly(hexamethylene adipate) (PHA), aminocaproic acid, hexamethylenediamine adipate (1:1), 6-aminiohexanoate, 1,4-butanediamine adipate, hexanedioic acid undecane-1,11-diamine, 6-diaminohexanoic acid, calcium bis(6-aminohexanoate), 6-aminohexanoyloxidanium, 6-aminohexanoic acid, octane-1,8-diamine hexanedioic acid, ethane-1,1,2-triamine hexanedioic acid, pentane-1,5-diamine heptanedioic acid, hexane-1,1-diamine hexanedioic acid, butane-1,1-diamine hexanedioic acid, (2S)-2,6-diaminohexanoic acid hexanedioic acid, heptane-1,6-diamine hexanedioic acid, 2-aminoacetic acid 2,6-diaminohexanoic acid, 2,6-diaminohexanoic acid hexanedioic acid, 2-aminoacetic acid (2S)-2,6-diaminohexanoic acid, cadaverine adipate, cadaverine adipate dihydrate, hexanedioic acid nonane-1,9-diamine, 6-aminocaproic acid-d6, 6-amino-2,2,6,6-tetradeuteriohexanoic acid, 6-aminohexanoic acid butanedioic acid, 6-amino-6,6-dideuteriohexanoic acid, 6-amino-2,2-dideuteriohexanoic acid, hexanedioic acid propan-1-amine, hexane-1,6-diamine octanedioic acid, 6-aminohexanoic acid hexanedioic acid, 6-(6-aminohexanoyloxy)-6-oxohexanoic acid, hexan-1-amine hexanedioic acid, 6-aminohexanoic acid carbamic acid, 2-aminoacetic acid 6-aminohexanoic acid, 6-aminohexanoic acid 3-aminopropanoic acid, ethane-1,2-diamine hexanedioic acid, 7-aminoheptanoic acid hexane-1,6-diamine, 6-aminohexanoic acid (2s)-2,6-diaminohexanoic acid, 6-aminohexanoic acid 3-aminopropanoic acid, butane-1,4-diamine heptanedioic acid, 6-amino-6-oxohexanoic acid hexane-1,6-diamine, acetic acid 6-aminohexanoic acid, 6-aminohexanoic acid azane, ethanamine hexanedioic acid, 4-aminobutanoic acid 6-aminohexanoic acid, 6-aminohexanoyl 6-aminohexaneperoxoate, 6-aminohexanoic acid ethane-1,2-diamine, 5-azaniumylpentylazanium hexanedioate, heptane-1,1-diamine hexanedioic acid, amino hexanoate 6-aminohexanoic acid, decane-1,10-diamine hexanedioic acid, ethane-1,2-diamine hexanedioic acid, 6-azaniumylhexylazanium hexanedioate hexanedioic acid, 6-(dideuterioamino)hexanoic acid, 7-aminoheptanoic acid 6-aminohexanoic acid, hexane-1,5-diamine hexanedioic acid, 6-aminohexanoic acid hexanoic acid, hexamethylendiammonium-adipat-dihydrat, tetramethylene diammonium adipate, 6-aminohexanoic acid hexane-1,6-diamine, 6-(4-aminobutylamino)oxy-6-oxohexanoic acid, hexane-1,6-diamine 6-hydroxyhexanoic acid, 6-azaniumylhexylazanium hexanedioate hydrate, 6-aminohexanoic acid hydrate, 6-aminohexanoic acid methylideneazanium, 6-aminohexanoic acid ethane molecular hydrogen, 6-aminohexanoic acid methanamine, heptane-1,7-diamine heptanedioic acid, 6-aminohexanoic acid ethane fermium cyanide, 5-carboxypentylazanide, 6-(6-aminohexylamino)oxy-6-oxohexanoic acid, 6-aminohexanoic acid zinc, octanedioic acid pentane-1,5-diamine, azanium 6-aminohexanoic acid, 6-aminohexanoic acid 2,6-diaminohexanoic acid, carbamic acid hexane-1,6-diamine hexanedioic acid, 6-(6-aminohexylamino)oxy-6-oxohexanoic acid methane, azanium 6-azaniumylhexylazanium hexanedioate, butan-1-amine hexanedioic acid, heptan-1-amine hexanedioic acid, 6-aminohexanoic acid 8-aminooctanoic acid, 7-aminoheptanoic acid heptanedioic acid, hexanedioic acid pentane-1,1-diamine, N-(6-aminohexyl)hydroxylamine hexanedioic acid, 6-azaniumylhexylazanium hexanedioic acid, 6-aminohexanoic acid ethane, 6-aminohexanoic acid hydrate, 7-azaniumylheptylazanium hexanedioate, butane-1,4-diamine hexanoic acid, 4-aminobutanoic acid;6-aminohexanoic acid;3-aminopropanoic acid, and 6-aminohexanoate.

In some embodiments, an adjunct 604 that has a hydroxy-terminated polymer chain can enhance the biodegradation profile. A stoichiometric excess of hydroxyl over carboxylic acid functionality ensures that the finished polymer is hydroxy-terminated, whereas a carboxyl excess leads to carboxylic acid-terminated polyesters. Branching can be incorporated into the polyester backbone using trifunctional monomers such as trimethylolpropane, 2,2-di(hydroxymethyl)-1-butanol, or trimellitic anhydride.

Polyether polyols are the most common type of polyols. Polyether polyols are made by the reaction of epoxides with compounds having active hydrogen atom. The typical polyester polyol. Polyether polyols have low glass transition temperature (T_{g}), which imparts good retention of physical properties and impact resistance at very low temperatures. In general polyether-based polyurethanes exhibit higher rebound (resilience) compared to polyester-based polyurethanes. Example polyether polyol monomers include, without limitation, polyethylene glycol, polytetramethylene ether glycol, polypropylene oxide glycol, polybutylene oxide glycol, triethylene glycol monoethyl ether, 1,2-diethoxyethane 2-[2-(2-methoxyethoxy)ethoxy]ethanol, 1,2-diethoxyethane 2-methoxyethanol, 2-[2-[2-(2-hydroxyethoxy)ethoxy]ethoxy]ethanol methane, 1-ethoxy-2-methoxyethane 2-methoxyethanol, methane 2-[2-(2-methoxyethoxy)ethoxy]ethanol hydrate, methanol 2-[2-(2-methoxyethoxy)ethoxy]ethanol, 2-(2-(2-(2-(2-(2-ethoxyethoxy)ethoxy)ethoxy)ethoxy)ethoxy)ethanol, 2-[2-(2-ethoxyethoxy)ethoxy]ethanol hydrate, 2,5,8,11-tetraoxatridecan-13-ol, 2-(2-{2-[2-(2-methoxy-ethoxy)-ethoxy]-ethoxy}-ethoxy)-ethanol, 2,5,8,11,14,17-hexaoxanonadecan-19-ol, 3,6,9,12-tetraoxatetradecan-1-ol, 3,6,9,12,15-pentaoxaheptadecan-1-ol, 4,7,10,13-tetraoxa-1-oxoniacyclopentadecane, pentaethylene glycol-water, 2-[2-[2-[2-(2-ethoxyethoxy)ethoxy]ethoxy]ethoxy]ethanol, butane ethane-1,2-diol 2-propoxyethanol, butane 2-ethoxyethanol, ethanol, 2,2′-[1,2-ethanediylbis(oxy)]bis-, mixt. with 2,2′-oxybis[ethanol], 3,6,9,12,15-pentaoxaoctadecan-1-ol, 3,6,9,12-tetraoxapentadecan-1-ol, 2-(2-(2-propoxyethoxy)ethoxy)ethanol, pentaethylene glycol, hexaethylene glycol, tetraethylene glycol, triethylene glycol monomethyl ether, sodium 1,4,7,10,13,16-hexaoxoniacyclooctadecane, sodium 1,4,7,10,13-pentaoxoniacyclopentadecane, sodium 1,4,7,10-tetraoxoniacyclododecane, potassium 1,4,7,10,13-pentaoxoniacyclopentadecane, ytterbium 2-methoxyethoxide, cerium 2-methoxyethoxide, 2-[2-[2-(2-propoxyethoxy)ethoxy]ethoxy]ethanol, neodymium methoxyethoxide, erbium methoxyethoxide, ethanol 2-(2-hydroxyethoxy)ethanol pentane, butyl carbitol water, 2-[2-[2-(2-hydroxyethoxy)ethoxy]ethoxy]ethyl hypofluorite, lanthanum methoxyethoxide, 2-[2-(2-butoxyethoxy)ethoxy]ethanol propane, potassium 1,4,7,10,13,16-hexaoxoniacyclooctadecane, 2-[2-[2-[2-(2-hydroxyethynoxy)ethoxy]ethoxy]ethoxy]ethanol, 3,6,9,12,15,18-hexaoxahenicosan-1-ol, triethylene glycol-water, yttrium methoxyethoxide, heptaethylene glycol monomethyl ether, 2-(2-(2-ethoxyethoxy)ethoxy), 3,6,9,12,15,18-hexaoxaicosane-1,20-diol, 3,6,9,12,15-pentaoxanonadecan-1-ol, 3,6,9,12-tetraoxahexadecan-1-ol, triethylene glycol monobutyl ether, and the like.

Adding portions of the adjunct 604 with varying degrees of hydrophilicity can allow the adjunct to undergo a phase change upon exposure to a fluid. As the hydrophilicity increases, the adjunct will move from surface to bulk degradation due to the increased water ingress. In some examples, as the fluid swells the network, a material having the same polarity as the polymer may pass through a lower critical solution temperature (LCST). In general, the LCST is the critical temperature below which the components of a mixture are miscible in all proportions. In some embodiments, the polymer system in solution has an LCST at a temperature higher than the upper critical solution temperature (UCST), meaning that there is a temperature interval of complete miscibility, with partial miscibility at both higher and lower temperatures. The UCST is the critical temperature above which the components of a mixture are miscible in all proportions.

For polymers in a solution or fluid, the LCST depends on the polymer's degree of polymerization, polydispersity, branching, composition, and architecture. At temperatures below the LCST, portions of the polymer in solution or fluid are completely miscible in all proportions, whereas above the LCST, partial liquid miscibility occurs.

In some embodiments, adjunct 604 can include certain segments of a block copolymer that have an LCST ranging from approximately 20° C. to approximately 35° C. This allows at least a portion of the adjunct to undergo a phase transition at lower temperatures compared to a polyurethane backbone. Upon passing through the LCST, one of the polar or non-polar portions of the adjunct can micellize and form effective crosslinks within the system. In this manner, the fluid content increase due to fluid ingress can cause the adjunct to undergo a phase transition from one to two phases and then back to one phase. During the one phase, miscible period, the adjunct 604 has a low mechanical strength, which is desirable during deployment. During the two phase, immiscible period, fluid is absorbed into the adjunct 604 and the mechanical strength increases due to the effective increase in crosslinking that counterbalances the breakdown of the adjunct 604. Once a critical level is reached, the crosslinks can be disrupted, and the mechanical strength of the adjunct will decrease. This phase change allows for both low deployment forces and faster absorbing adjuncts since the critical phase of the healing process has reversible crosslinks.

In some embodiments, the second polymerizable compound can be a poloxamer added into the backbone of the polyurethane. Poloxamers are a class of water-soluble nonionic triblock copolymers formed by polar (polyethylene oxide) and non-polar (polypropylene oxide) blocks. Poloxamers can result in amphiphilic and surface-active properties to the polymer backbone. In some embodiments, increasing the water ingress into the polar portion of the adjunct 604 can promote faster absorption kinetics. The LCST is dependent on the polypropylene oxide segment length and relative water content.

In any of the embodiments described herein, the second polymerizable compound can be added as pendant groups to the first polymerizable compound backbone such that the second polymerizable compound forms a coating. The coating can act to prevent or delay the fluid ingress to the first polymerizable compound.

In some embodiments, the second polymerizable compound can act as an enteric coating in the presence of gastric fluid, or near an infection. The coating can be resistant or substantially resistant to absorption at certain pH, such as, for example, acidic conditions (pH<7) such that the coating can be used for tissue at or near the stomach. In other examples, the coating can delay absorption time for a predetermined timeframe and prevent the bulk material of the adjunct from contacting fluids. Gastric and infected fluids tend to have a lower pH than other locations in the body. Using an adjunct 604 with a second polymerizable compound that is resistant to degradation under acidic conditions can be beneficial when treating tissue near stomach, gastrointestinal tract, or other acidic areas of the body. In general, the retention of mechanical strength of the adjunct 604 is a function of absorption and fluid ingress. The second polymerizable compound can prevent or slow fluid penetration at lower pH's. As the pH increases, fluid is allowed to pass through the coating and initiate the degradation of the first polymerizable compound. The second polymerizable compound can be tailored to have a specific induction period based on pH by adjusting the composition, thickness, branching, and the like.

In some embodiments, the second polymerizable compound can be a polymethacrylate based copolymer with specific carboxylic acid functionalization. In addition, the second polymerizable compound can also include as a copolymer with or without the polymethacrylate, without limitation, a polydioxanone, a polyanhydride, a hydroxypropyl methylcellulose acetate succinate, a cellulose acetate phthalate, a cellulose acetate trimellitate, a hydroxypropyl methylcellulose phthalate, a polyvinyl acetate phthalate, a poly(trimethylene carbonate), a poly(beta-thioether ester ketal), a polypropylene fumarate, a poly(ester urea), a poly(ester amide), or combinations thereof.

In addition to a single enteric coating layer, the second polymerizable compound also forms a multilayer construct to provide additional advantages independent of the local pH environment. In some embodiments, the second polymerizable compound can form an acid-generating surface of the adjunct such that the enteric coating is maintained as additional protons or acid species are generated. This would extend the induction period provided by the enteric coating and further prevent any fluid ingress. As the surface eroding polymer degrades to a critical point, such as when the enteric coating is in the presence of a neutral pH, fluid ingress may proceed and allow for stapling implant absorption. In some embodiments, a multilayer construct may exist as a bilayer. In any embodiment described herein, the bilayer or multilayer construct can include active pharmaceutical ingredients (API's) that could be released as the surface eroding polymer degrades. Example acid-generating polymers can include, without limitation, polyanhydrides such as poly(carboxyphenoxy hexane-sebacic acid), poly(fumaric acid-sebacic acid), poly(imide-sebacic acid), and poly(imide-carboxyphenoxy hexane). Other acid-generating polymers can include 3-iodopropyl acetal moieties.

In some embodiments, when the second polymerizable compound forms a coating, the composition and/or thickness of the coating can provide an induction period for the absorption and/or degradation process. The initial thickness of the second polymerizable compound coating prior to delivery and/or an induction period can range from about 20 μm to about 100 μm (e.g., from about 30 μm to about 90 μm, from about 40 μm to about 60 μm, from about 45 μm to about 50 μm, and any range in between). Upon exposure to a temperature or a pH, the thickness of the second polymerizable compound coating may increase depending on the compression of the tissue required to reach a hemostatic seal.

As would be appreciated by one of skill in the art, the addition of the second polymerizable compound can be done with spatial control for optimal absorption, tissue in-growth, and/or hemostatic behavior. Further, the composition of the first and second polymerizable compound can be a gradient along a portion of the adjunct 604 (e.g., along a length, at a central point, at the ends, along the staple line, etc.,) with a varying bio-absorption profile. In general, a short-term absorption profile may be preferred to address hemostasis while a long-term absorption profile may address better tissue healing without leakages.

In some embodiments, adding the second polymerizable compound to the backbone, as pendant groups to the first polymerizable compound, or as a coating, can be done through any suitable technique including, without limitation, inkjet printing, direct deposition, thermal spraying, cold dynamic spraying, cold spraying, electro spraying, ultrasonic spray coating, dip coating, screen printing, spin coating, solution deposition, stereolithography, exterior lamination, and the like.

In some embodiments, the change in crosslink density between the first polymerizable compound and the second polymerizable compound forms a gradient of compression strength along a portion of the adjunct 604. In general, the adjunct 604 may have a compression strength of about 20 kPa to about 70 kPa, such as about 30 kPa to about 60 kPa (e.g., about 42 kPa), about 30 kPa to about 50 kPa, about 32.5 kPa to about 37.5 kPa. In some embodiments, the adjunct 604 may have a compression strength of about 20 kPa to about 70 kPa due to a portion of the adjunct 604 having the first polymerizable compound, and a compression strength of about 15 kPa to about 50 kPa due to a portion of the adjunct 604 having the second polymerizable compound.

In some examples, the adjunct 604 may have a tensile strength of about 30 kPa to about 90 kPa such as about 45 kPa to about 85 kPa or about 55 kPa to about 75 kPa. In some examples, the adjunct 604 will have tensile strength of about 110 kPa to about 150 kPa.

In any of the embodiments disclosed herein, the adjunct having a block copolymer having the first polymerizable compound and the second polymerizable compound can modulate the degradation profile of the adjunct (and the mechanical properties of the bioabsorbable material) for a predetermined time frame. For instance, a phase change may occur over several seconds or minutes such that during delivery, the porous body is readily compressible, and the end effector is easily inserted through the trocar. After the induction period passes, the mechanical strength of the adjunct 604 increases to allow for a suitable hemostatic seal between the tissue. This increased mechanical strength after the induction period can range from approximately 10 minutes to approximately 6 weeks, the amount of time that may be necessary for the tissue to heal.

Turning back to FIG. 9A, the end effector 106 is shown in a delivery configuration, where the adjunct 604 is compressed between the anvil 102 and the cartridge 200. As illustrated, the end effector 106 can include a staple cartridge 200 and an adjunct 604 comprising a bioabsorbable material. In certain embodiments, the adjunct 604 is releasably retained on the cartridge 200. When the end effector 106 closes around tissue T, the adjunct 604 can compress from a thickness of the uncompressed adjunct UT to a thickness of compressed adjunct CT of adjunct in response to a variation in tissue thickness.

FIG. 9B is a side view of the end effector 106 after firing of staples 300, where the adjunct 604 is no longer compressed between the anvil 102 and the cartridge 200. As shown, the adjunct 604 is released from the cartridge 200 after firing. Adjunct 604 is shown to maintain the thickness of the compressed adjunct CT and the thickness of uncompressed adjunct UT from pre-firing in FIG. 9A. In some embodiments, the adjunct 604 can undergo a phase change before firing of staples such that the adjunct height UT and compressed portion CT will be maintained after firing and releasing adjunct 604 and tissue T. In other embodiments, the adjunct 604 can undergo phase changes after a predetermined timeframe, as described above. In such an example, the adjunct height UT and compressed portion CT can either expand or contract in response to the staple height and the tissue thickness. As would be appreciated by those of skill in the art, a phase change of the adjunct 604 upon exposure to fluid ingress can be advantageous to ensure a hemostatic seal during changes in tissue inflammatory responses and throughout the healing process.

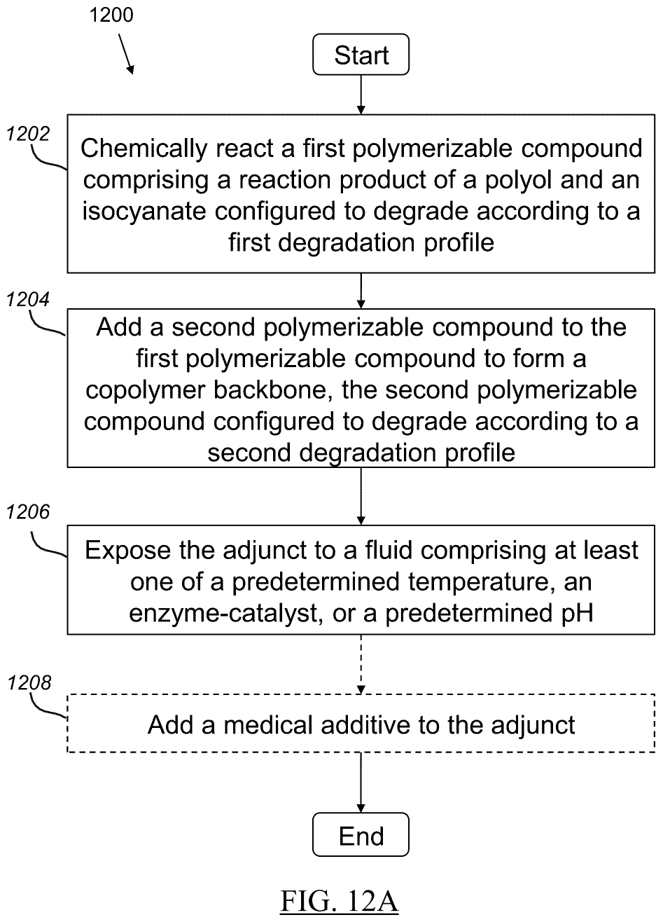

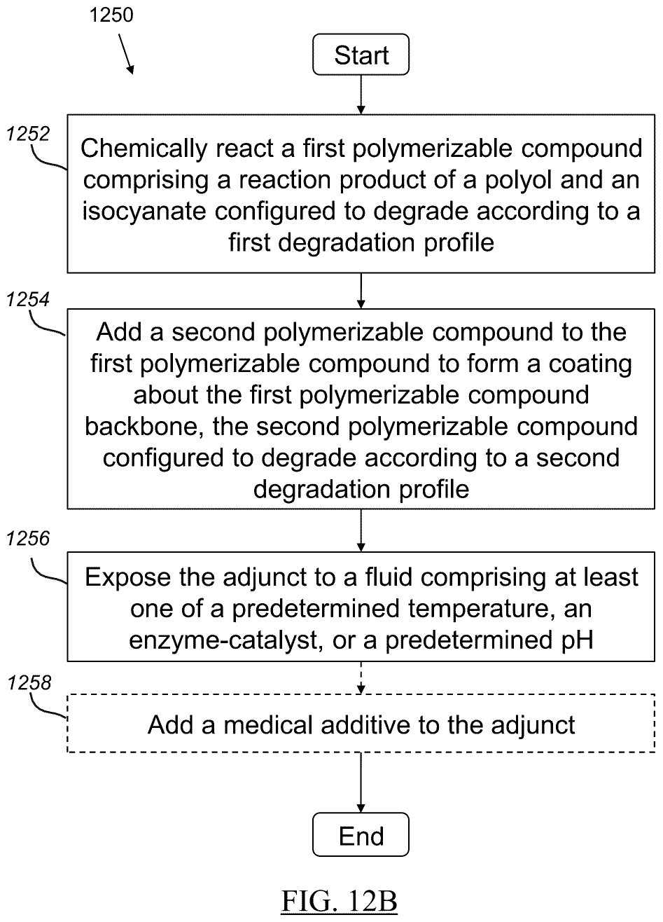

FIGS. 12A and 12B are flowcharts of methods 1200 and 1225 of forming the surgical adjunct 604, including a bioabsorbable material. The techniques for tuning the absorption pathway and changing mechanical properties described herein may offer an added benefit of increasing adjunct strength and durability in combination with positive in vivo interactions (e.g., biocompatibility, wound healing, tissue integration, chemotherapy, anti-inflammatory, bone growth and integration, ligament and tendon repair, etc.) when the adjunct is delivered to the tissue site, as described herein. As such, the techniques described herein may allow the bioabsorbable material itself to aid in the healing process of the surrounding tissue. In addition, the embedding techniques described herein may offer an added benefit of preventing fibrous encapsulation of the foam cushion, and/or providing tunable release profiles for a variety of medical additives delivered to the tissue site.

Specifically with respect to FIG. 12A, method 1200 used for tuning the absorption pathways in a bioabsorbable material (e.g., a foam) may include chemically reacting a first polymerizable compound comprising a reaction product of a polyol and an isocyanate configured to degrade according to a first degradation profile (step 1202). In some examples, the first polymerizable compound may include a polyurethane, a polyester urethane, a polyester urea, a polyester, a polycarbonate, a polyorthoester, a polyanhydride, a polyesteramide, a polyphosphazenes, a polyphosphoesters, a polysaccharides, and/or a polyoxaester. Method 1200 also includes adding a second polymerizable compound to the first polymerizable compound to form a copolymer backbone with the first polymerizable compound. (step 1204). The second polymerizable compound is configured to degrade according to a second degradation profile. After introduction of the second polymerizable compound, method 1200 includes exposing the adjunct to a fluid comprising at least one of a predetermined temperature (higher or lower), an enzyme-catalyst, or a predetermined pH (step 1206). Method 1200 can end after step 1206 or can optionally include adding a medical additive to the porous body (optional step 1208). In such examples, the medical additives may include medicants to treat pain and/or promote wound healing, tissue growth, infection reduction, and the like.

FIG. 12B, similar to FIG. 12A, includes a method 1250 used for tuning the absorption pathways in a bioabsorbable material (e.g., a foam), but instead of forming a backbone including both the first polymerizable compound and the second polymerizable compound of step 1204, method 1250 includes adding the second polymerizable compound to the first polymerizable compound to form a coating about the backbone (step 1254). The coating can be resistant or substantially resistant to absorption at certain pH, such as, for example, acidic conditions (pH<7) such that the coating can be used for tissue at or near the stomach. In other examples, the coating can delay absorption time for a predetermined timeframe and prevent the bulk material of the adjunct from contacting fluids. The first polymerizable compound can form the backbone of the bioabsorbable material. As described above, the first polymerizable compound may include a polyurethane, a polyester urethane, a polyester urea, a polyester, a polycarbonate, a polyorthoester, a polyanhydride, a polyesteramide, a polyphosphazenes, a polyphosphoesters, a polysaccharides, and/or a polyoxaester. The second polymerizable compound is configured to degrade according to a second degradation profile. After introduction of the second polymerizable compound, method 1250 includes exposing the adjunct to a fluid comprising at least one of a predetermined temperature, an enzyme-catalyst, or a predetermined pH (step 1256). The second polymerizable compound can prevent or slow fluid penetration at lower pH's. As the pH increases, fluid is allowed to pass through the coating and initiate the degradation of the first polymerizable compound. Method 1250 can end after step 1256 or can optionally include adding a medical additive to the porous body (optional step 1258).

### Spatial Control of Mechanical Properties Through Selective Crosslinking Density

As described above, the end effector 106 (shown in FIG. 1) comprising the cartridge 200 and adjunct 604 is closed or substantially closed for inserting through a trocar to the delivery site. During delivery, the surgical adjunct 604 needs to have mechanical properties such that the material is sufficiently compressible, but must have increased strength to retain staples, sutures, and screws at the delivery site. The surgical adjunct 604 can be tuned for the particular purpose before, during, and after surgical procedures and have one or more of the properties described below that modulate the mechanical properties. In particular, the adjunct 604 described has selective crosslinking density control in vivo and can also have a certain compressibility when attached to the cartridge outside of the body.

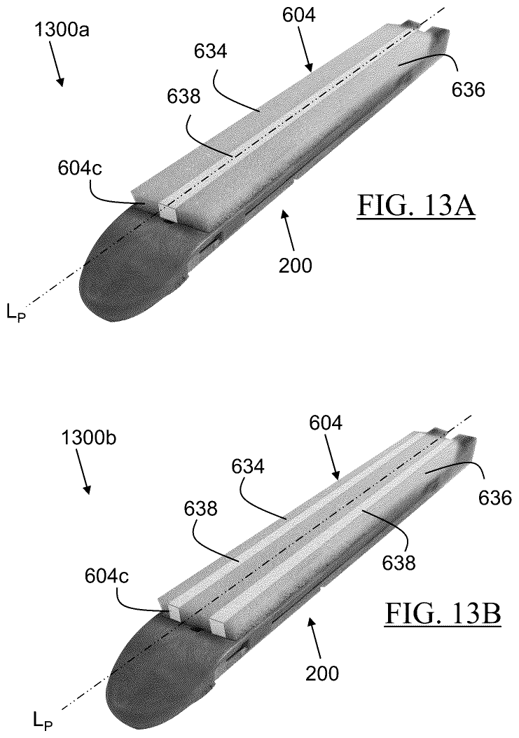

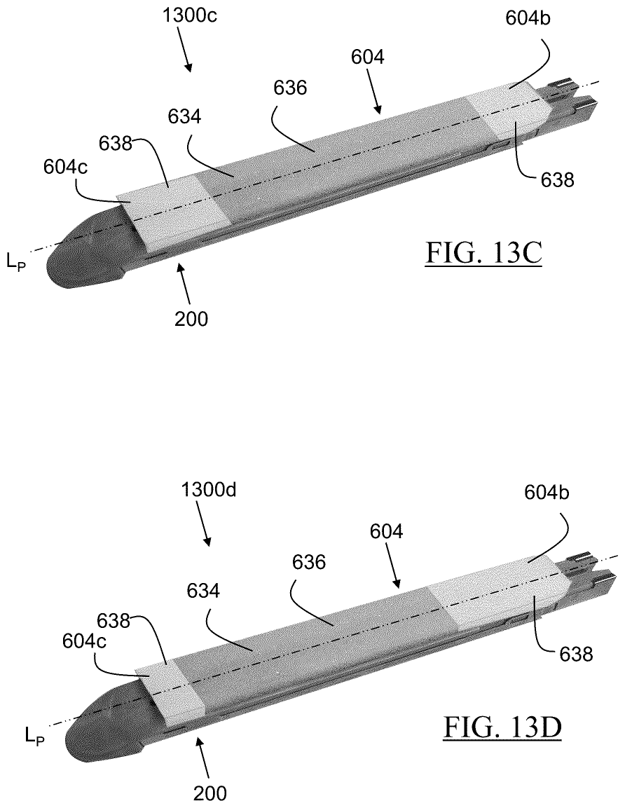

Turning to FIGS. 13A and 13B, example staple cartridge assemblies 1300a, 1300b with spatial control of mechanical properties include an adjunct 604 having a first zone 636 and a second zone 638. The first zone 636 of the porous body 634 includes a first crosslink density, while the second zone 638 has a second crosslink density different than the first zone 636. As shown in the staple cartridge assembly 1300a, the second zone 638 can be positioned approximately central along a longitudinal axis Lₚ of the porous body 634. Another orientation of spatial control of a cartridge assembly 1300b is provided in FIG. 13B, where more than one second zone 638 is positioned along the longitudinal axis to provide a gradient of crosslink density laterally. As shown in FIG. 13B, the second zone 638 can be positioned centrally along each half of the adjunct 604. In some examples, the spatial control shown in FIG. 13B may provide for an optional slot/slit 808 separating or partially separating two parallel portions of the adjunct 604, as shown in FIG. 8.

As would be appreciated by one of skill in the art, such spatial control could be more optimal for tissue in-growth or hemostatic behavior. Further, the gradient could also be compositional with a varying bio-absorption profile. A short-term absorption profile may be preferred to address hemostasis while a long-term absorption profile may address better tissue healing without leakages.

FIGS. 13C and 13D provide example staple cartridge assemblies 1300c, 1300d with spatial control of mechanical properties include an adjunct 604 having a first zone 636 and a second zone 638 positioned at approximately opposing ends of the adjunct 604, such as at the proximal end 604c and a distal end 604b. In some embodiments, the second zone 638 at the proximal end 604c and distal end 604b of the adjunct 604 can be substantially equal in length, as illustrated in FIG. 13C. Alternatively, the second zone 638 can be substantially greater in length at the distal end 604b of adjunct 604, where it may be desirable to modulate the crosslink density where the compression between the anvil and the cartridge 200 initiates. Although not depicted, an orientation where the second zone is substantially greater in length at the proximal end 604c of the adjunct 604 is also contemplated. In some examples, the spatial control shown in FIGS. 13C and 13D may provide for an optional slot/slit 808 separating or partially separating two parallel portions of the adjunct 604, as shown in FIG. 8.

In some embodiments, an example staple cartridge assembly with spatial control of mechanical properties can include an adjunct 604 having a first zone 636 and a second zone 638, where the second zone 638 is positioned over at least a portion of one side of the first zone 636. For instance, the second zone 638 can coat over the first zone 636 on at least one side, or alternatively on two sides, or all sides. The coating can be resistant or substantially resistant to absorption at certain pH, such as, for example, acidic conditions (pH<7) such that the coating can be used for tissue at or near the stomach. In other examples, the coating can delay absorption time for a predetermined timeframe and prevent the bulk material of the adjunct from contacting fluids.

The adjunct 604 has a porous body 634 made of at least one polymer that can undergo selective crosslinking via photocurable networks, monomer incorporation, stimuli responsive functional groups, and the like. The polymer may include polyurethane, polyester, polycarbonate, polyorthoester, polyanhydride, polyesteramide, and/or polyoxaester.

To achieve control of crosslink density, the polymer of the porous body 634 can have or be mixed with a polymerizable compound having functional groups that act as crosslinkable units.

In some embodiments, the polymerizable compound is a chain extender that does not contribute to crosslinking. The chain extender can be incorporated in the polymer's monomer backbone or as pendent groups. In some examples, the chain extender is carbon-carbon based and has double bonds that incorporate within the polymer backbone. As a non-limiting example, a fumaric acid can be added during the condensation reaction of the polymer backbone of the porous body. With the addition of a photoinitiator, the fumaric acid in the polymer backbone would provide a crosslinking site that is specifically controlled by lithography through the photocurable site. In addition, upon degradation of the porous body, such as in the body after a surgical procedure, dimethyl fumarate could be selectively generated from the polymer backbone. Dimethyl fumarate is a known Nrf2 activator that can be used to secondarily treat inflammation and prevent nerve damage.

In another example, the chain extender replaces one or more of the functional groups on the polymer backbone such that the crosslinking is reduced. For instance, in a polyurethane polymer backbone, a chain extender such as acrylate or methacrylate can replace one of the hydroxyl groups from the polyol. The chain extender can be fumaric acid, succinic acid, maleic acid, and/or combinations thereof.

Returning to FIGS. 13A-13B, the polymerizable compound is added only to the first zone 636 or only to the second zone 638 such that the porous body 643 has differing crosslink densities around the middle row of staples, or around staple pockets and between rows of staples. As would be appreciated by one of skill in the art, utilizing a lower crosslink density in the middle row, where the amount of pressure is lowest compared to the first and third rows, can result in a lower force required to fire the staples while maintaining the necessary compressive properties across the entire staple line.

Alternatively, the polymerizable compound can be added to both the first zone 636 and second zone 638, but in different concentrations. In another embodiment, polymerizable compounds having different functional groups and crosslink density can be added to one of the first zone 636 or the second zone 638. In some embodiments, the polymerizable compounds can be added before, during, or after the formation of the porous body.

In some embodiments, the change in crosslink density between the first zone 636 and second zone 638 forms a gradient of compression strength along a portion of the porous body 634. In general, the adjunct 604 may have a compression strength of about 30 kPa to about 70 kPa, such as about 30 kPa to about 60 kPa (e.g., about 42 kPa), about 30 kPa to about 50 kPa, about 32.5 kPa to about 37.5 kPa. In some embodiments, the adjunct 604 may have a compression strength of about 30 kPa to about 70 kPa in the first zone, and a compression strength of about 15 to about 50 kPa in the second zone. In order to test compression strengths, an adjunct 604 was placed in a humid warm environment at approximately 37° C., compressed to a first height, then a second height shorter than the first height, and then released back to the first height at which point the adjunct's compression strength was measured.

In some examples, the adjunct 604 may have a (peak) tensile strength of about 50 kPa to about 150 kPa or about 30 kPa to about 90 kPa such as about 45 kPa to about 85 kPa or about 55 kPa to about 75 kPa. In some examples, the adjunct 604 will have (peak) tensile strength of about 110 kPa to about 150 kPa. Tensile strength is measured on an adjunct 604 having the dog-bone configuration shown and described with respect to FIGS. 17A and 17B. Specifically, the adjunct's 604 tensile strength is measured after submerging it in water at a temperature of about 37° C. for less than a minute and then running a tensile strength test.

In some embodiments, the polymerizable compound is a crosslinking monomer added to the polymer backbone that is specifically undercured and retains reactive sites within the network. Additional crosslinking monomers can be deposited or otherwise applied onto or within the porous body in patterned locations to increase the mechanical properties locally. In one non-limiting example, a polymer backbone having isocyanate functional groups, a polyol crosslinking monomer can be added to increase crosslinking at the specific addition location, for example, along only one of the first zone 636 or second zone 638, as shown in FIGS. 13A and 13B. Because monofunctional alcohols act as capping agents to isocyanate functional groups, a monofunctional alcohol can be added to the zone intended to maintain the crosslink density. The crosslinking monomer can be an arylboronic acid, a styrylpyrene, a styrene, and combinations thereof.

In some embodiments, direct crosslinking monomer addition can be done through any suitable technique including, without limitation, inkjet printing, direct deposition, thermal spraying, cold dynamic spraying, cold spraying, electro spraying, ultrasonic spray coating, dip coating, screen printing, spin coating, and the like.

In yet another embodiment, the polymerizable compound is a stimuli-responsive functional group that can undergo reversible crosslinking upon exposure to one or more of heat, light, ultrasound waves, pH, counter ion exchange, and combinations thereof. Example stimuli-responsive functional groups include o-nitrobenzyl, coumarin, anthracene, disulfide, diselenide, Diels-Alder (diene and dienophile). Stimuli-responsive functional groups can allow for crosslink density transition from linear to complex architectures such as star, cyclic, or hyperbranched. The stimuli-responsive functional groups can include a vinyl acetate, an acrylonitrile, a vinylidene dichloride, an isoprene, a butadiene, a chloroprene, and/or combinations thereof.

In any of the embodiments disclosed herein, the polymerizable compound can modulate the crosslink density (and the mechanical properties of porous body) for a predetermined time frame. For instance, a crosslink density change may occur over several seconds or minutes such that upon delivery, the porous body is readily compressible, and the end effector is easily inserted through the trocar and after the induction period passes, the mechanical strength of the porous body increases in specific zones as a function of spatial arrangements (e.g., the first zone 636 of FIG. 13A).

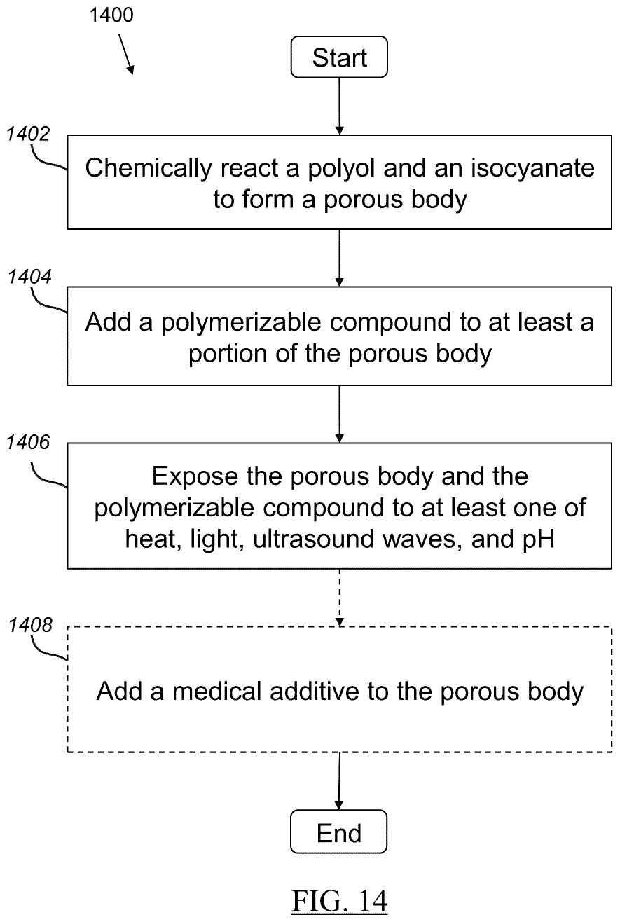

FIG. 14 is a flowchart of a method 1400 of forming surgical adjunct 604, including a bioabsorbable material that itself includes a porous body. The techniques for spatial control of mechanical properties described herein may offer an added benefit of increasing adjunct strength and durability in combination with positive in vivo interactions (e.g., biocompatibility, wound healing, tissue integration, chemotherapy, anti-inflammatory, bone growth and integration, ligament and tendon repair, etc.) when the adjunct is delivered to the tissue site, as described herein. As such, the techniques described herein may allow the bioabsorbable material itself to aid in the healing process of the surrounding tissue. In addition, the embedding techniques described herein may offer an added benefit of preventing fibrous encapsulation of the foam cushion, and/or providing tunable release profiles for a variety of medical additives delivered to the tissue site.

Specifically with respect to FIG. 14, the method 1400 used for spatial control of mechanical properties in a bioabsorbable material (e.g., a foam) may include chemically reacting a polyol and an isocyanate to form a porous body (step 1402). In some examples, the bioabsorbable material may include a polyurethane, polyether urethane, polyester urethane, polyester urea, polyester, polycarbonate, polyorthoester, polyanhydride, polyesteramide, polyphosphazenes, polyphosphoesters, polysaccharides, and/or polyoxaester. Method 1400 also includes adding a polymerizable compound to at least a portion of the porous body (step 1404). The polymerizable compound is at least one of a chain extender and a photoinitiator. After introduction of the polymerizable compound, method 1400 includes exposing the porous body and the polymerizable compound to at least one of heat, light, ultrasound waves, and pH (step 1406). Method 1400 can end after step 1406 or can optionally include adding a medical additive to the porous body (optional step 1408). In such examples, the medical additives may include medicants to treat pain and/or promote wound healing, tissue growth, infection reduction, and the like.

### Compressible Surgical Adjuncts, Cartridges, and Cartridge Assemblies

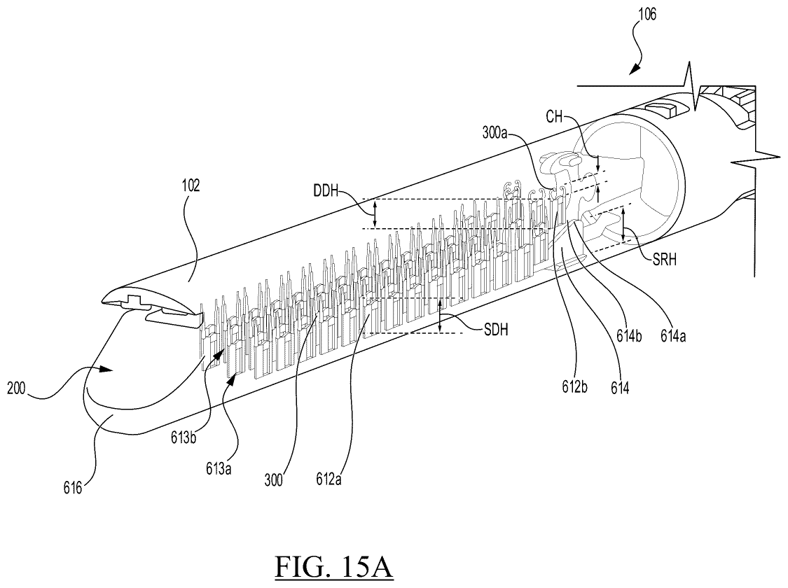

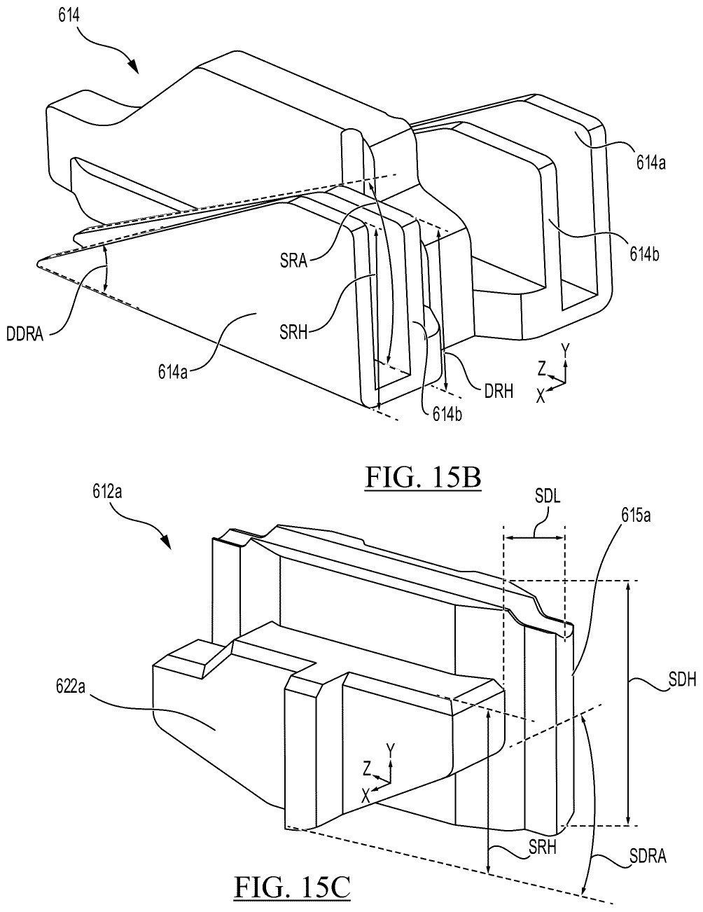

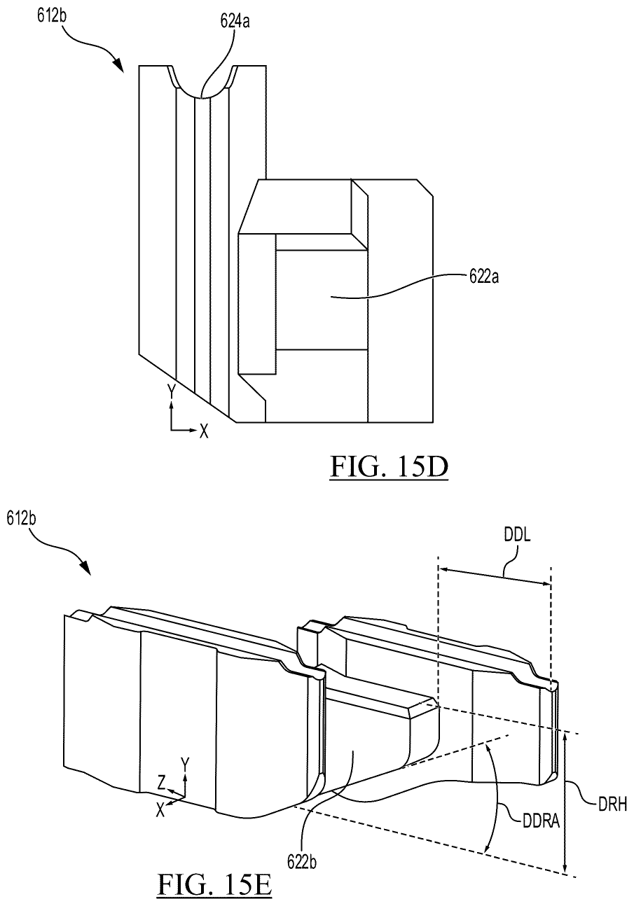

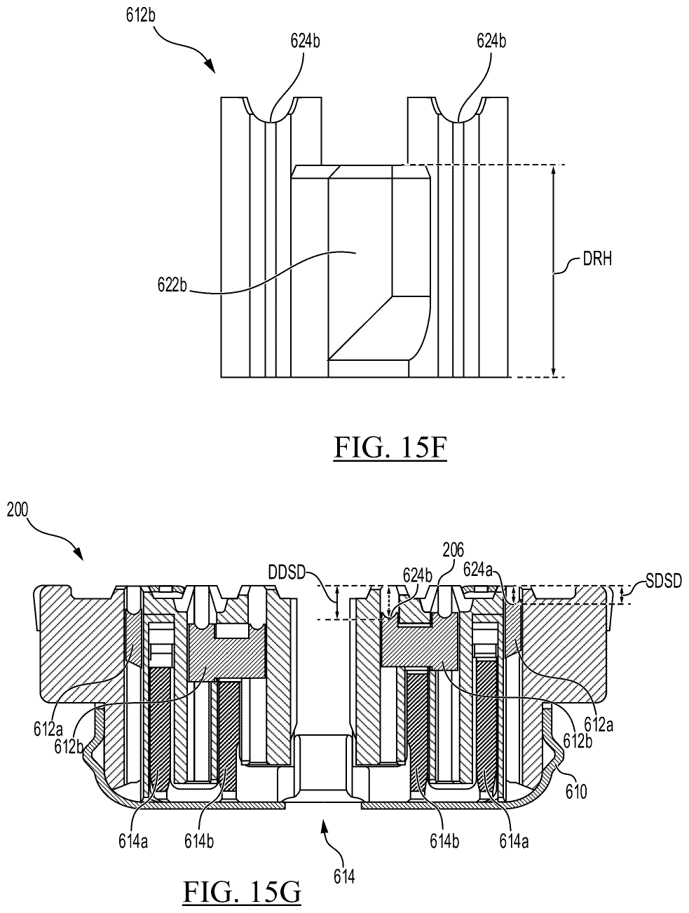

Turning to FIG. 15A, the staple cartridge 200 includes a sled 614 and a plurality of drivers 612 configured to drive one or more staples in an upward direction to deploy the staples when a user presses a firing trigger 122 shown in FIG. 1. Once the firing trigger 122 is pressed, the sled 614 moves toward the distal end 616 of the end effector 106 contacting one or more drivers 612a, 612b at a time forcing the one or more drivers 612a, 612b upward along with one or more corresponding staples 300 upward to form fired staple 300a and to capture a material, such as tissue (T), (see FIG. 7A.) between the anvil 104 and the fired staple 300a. The cartridge 200 may include a first row 613a of single drivers 612a that corresponds to driving staples 300 positioned in the third row of staple cavities 212c, 214c and a second row 613b of double drivers 612b that corresponds to driving staples 300 positioned in a first row of staple cavities 212a, 214b and a third row 212a, 212b (see FIG. 2A for staple cavities).

Referring to FIGS. 15A-15G, one or more single drivers 612a may have a height SDH of about 0.044 inches to about 0.074 inches, such as about 0.050 inches to about 0.068 inches, about 0.054 inches, or about 0.06 inches. One or more double drivers 612b may have a height DDH of about 0.044 inches to about 0.074 inches, such as about 0.050 inches to about 0.068 inches, about 0.054 inches, or about 0.06 inches. The sled 614 may have a least a first rail 614a corresponding to the single drivers 612a positioned in the first row 613a and a second rail 614b corresponding to the double drivers 612b positioned in the second row 613b. The first rail 614a may have a rail height SRH of about 0.164 inches or about 0.167 inches and engages with the single driver 612a. The second rail 614b may have a rail height DRH of about 0.140 inches to about 0.162 inches such as about 0.149 inches or about 0.152 inches and engages with the double driver 612b. Once the staples 300 are deployed, they form a fired staple 300a with a crush height CH of about 0.08 inches to about 0.12 inches such as about 0.97 inches or about 0.1 inches.

Referring to FIG. 15G, the saddles 624a of single drivers 612a may be spaced apart from the cartridge deck 206 a distance SDSD when the single driver 612a is fully engaged with the sled 614 of about −0.010 inches to about −0.031 inches such as −0.021 inches or about −0.017 inches. The saddles 624b of the double driver 612b may be spaced apart from the cartridge deck 206 a distance DDSD when the double driver 612b is fully engaged with the sled 614 about −0.022 inches to about −0.042 inches such as about −0.032 inches or about −0.036 inches.

Referring to FIGS. 15B-15G, one or more single drivers 612a may have a rigger 622a that may have a ramp location SDL that extends about 0.056 inches to about 0.066 inches, such as about 0.061 inches, from a proximal edge 615a of the single driver 612a. Rigger 622a may have a height SRH of about 0.035 inches to about 0.055 inches such as about 0.45 inches or about 0.43 inches. One or more double drivers 612b may have a rigger 622b that may have a ramp location DDL that extends 0.094 inches to about 0.114 inches, such as about 0.104 inches, from the proximal edge 615b of the double driver 612b. Rigger 622b may have a height of about 0.039 inches to about 0.060 inches such as about 0.050 inches or about 0.048 inches. The single driver 612a, may have a ramp angle SDRA of about 24 degrees to about 34 degrees, such as about 29 degrees. The double driver 612b may have a ramp angle DDRA of about 22 degrees to about 30 degrees, such as about 26 degrees. The sled's 614 first rail 614a may have a ramp angle SRA of about 22 to about 32 degrees, such as about 26.9 degrees. The sled's 614 second rail 614b may have a ramp angle SRA of about of about 20 degrees to about 30 degrees, such as about 24.1 to about 24.5 degrees.

In use, once the surgical stapling and severing device, like device 100 in FIG. 1, is directed to the surgical site, tissue is positioned between the anvil 102 and the stapling assembly 600 such that the anvil 102 is positioned adjacent to a first side of the tissue and the stapling assembly 600 is positioned adjacent to a second side of the tissue (e.g., the tissue can be positioned against the tissue-contacting surface 604a of the adjunct 604). Once tissue is positioned between the anvil 102 and the stapling assembly 600, the surgical stapler can be actuated, e.g., as discussed above, to thereby clamp the tissue between the anvil 102 and the stapling assembly 600 (e.g., between the tissue-compression surface 102a of the anvil 102 and the tissue-contacting surface 604a of the adjunct 604) and to deploy staples from the cartridge through the adjunct and into the tissue to staple and attach the adjunct to the tissue.

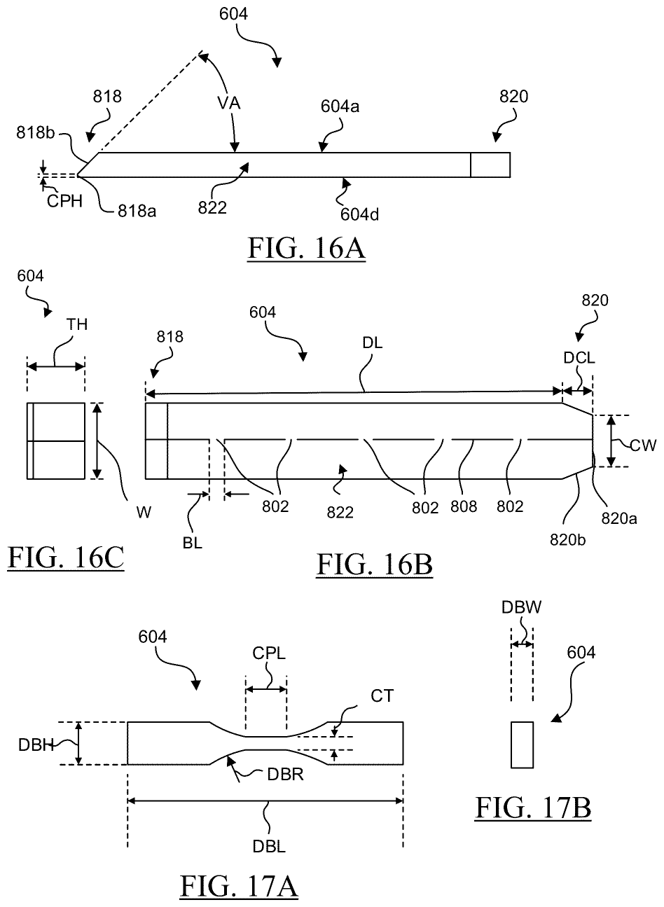

Referring to FIGS. 16A-16C, the adjunct 604 has a lower surface 604d and may have a distal chamfered portion 818, a proximal chamfered portion 820, and a center portion 822. The distal chamfered portion 818 has a vertical portion 818a having a height CPH of about 0.009 inches to about 0.029 inches, such as about 0.019 inches. The distal chamfered portion 818 may have an angled portion 818b proximal the vertical portion 818a. The angled portion 818b has a slope VA of about 30 degrees to about 60 degrees, such as about 45 degrees, measured from the tissue contacting surface 604a.

Referring to FIG. 16A, the distal chamfered portion 818 and the center portion 822 has a combined length DL of about 2.25 inches to about 2.45 inches, such as about 2.35 inches. The proximal chamfered portion 820 has an angled portion 820b with a length DCL of about 0.1 inches to about 0.3 inches. In addition, the proximal chamfered portion 820 has a horizontal portion 820a and an angled portion 820n. The horizontal portion 820a may have a width CW of about 0.27 inches to about 0.29 inches, such as about 0.28 inches.

In some examples, the adjunct 604 includes one or more slits 808 with two or more bridges 802 spaced apart by a bridge length BL of about 0.035 inches to about 0.045 inches such as about 0.04 inches.

As previously mentioned, the adjunct 604 is compressible. FIGS. 17A and 17B show the dog-bone shape and dimensions that the adjunct 604 takes when subjected to compressibility tests to determine its material properties. As shown, the adjunct 604 has a compression length CPL of about 9.45 mm to about 9.61 mm, such as about 9.53 mm. The adjunct 604 has a compression thickness CT of about 3.15 mm to about 3.21 mm, such as about 3.18 mm. The adjunct 604 has dog bone radius DBR of about 12.62 mm to about 12.78 mm, such as about 12.7 degrees. The adjunct 604 has a dog bone height DBH of about 6.35 mm to about 12.71 mm, such as about 9.53 mm. The adjunct 604 has a dog bone length DBL of about 40 mm to about 80 mm, such as about 63.5 mm. The adjunct has a dog-bone width DBW of about 2.5 mm to about 3.5 mm, such as about 3.03 mm.

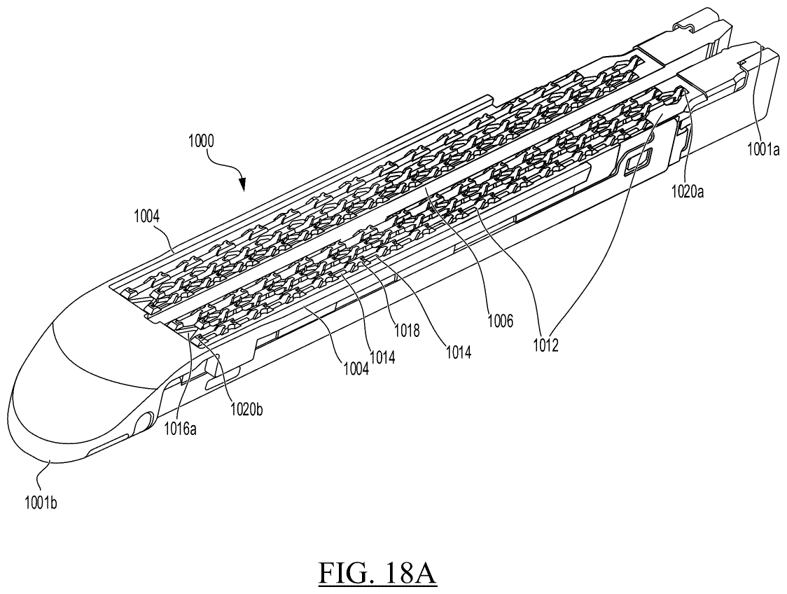

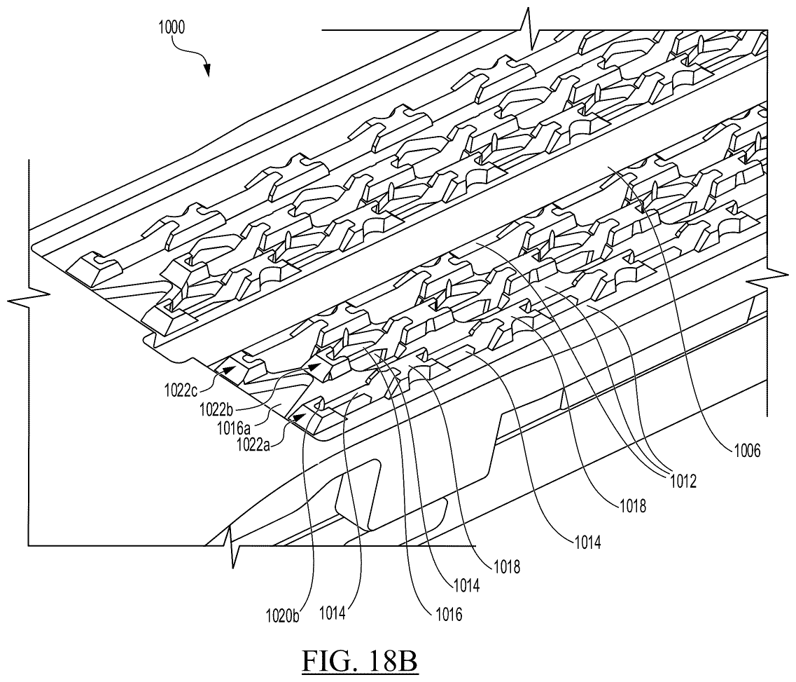

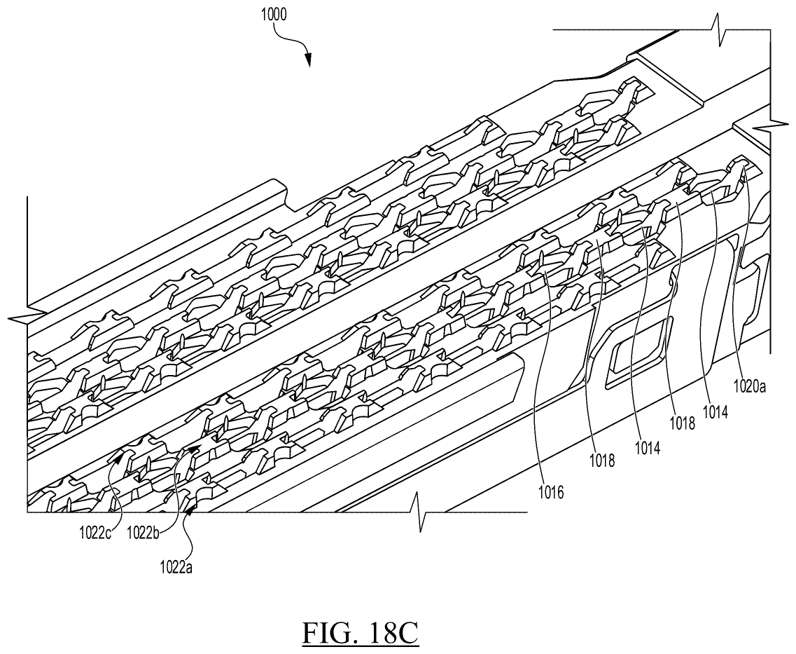

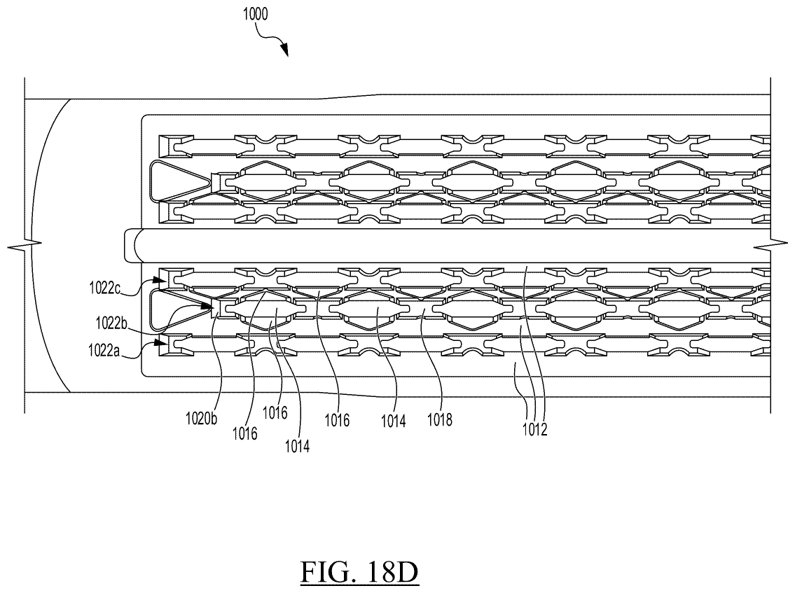

FIGS. 18A-18D illustrate an exemplary staple cartridge 1000 with a deck 1012 having three levels so that the adjunct 604 may expand around the various protrusions and recessions to increase the contact surface area between the adjunct 604 and the staple cartridge 1000 to increase the adherence and alignment between the staple cartridge 1000 the adjunct 604. Staple cartridge 1000 is similar to staple cartridge 200, but with more exemplary details showing the deck 1012. In particular, the staple cartridge 1000 may include a distal end 1001b having an atraumatic shape and a proximal end 1001a. The staple cartridge 1000 may include one or more raised ledges 1004 (such as two raised ledges 104) aligning with longitudinal edges of the deck 1012. The staple cartridge 1000 may include a plurality of staple cavities 1014 positioned and aligned in a first row 1022a, a second row 1022b, and a third row 100c on each side of cartridge slot 1006. In each row, at least two adjacent staple cavities 1014 are separated by a raised surface 1018 or combined raised surface of the deck 1012 that conforms to the ends of the staple cavities 1014. This combined raised surface 1018 essential combines a raise surface corresponding proximal end of an adjacent staple cavity 1014 with a distal end of a staple cavity 1014. In addition, staple cavities 1014 positioned at the distal end 1001b and the proximal end 1001a of the first row 1022a, the second row 1022b, and the third row 100c include raised surfaces 1020b, 1020a that are approximately half the size of the combined raised surfaces 1018 between staple cavities 1014. The cartridge deck 1012 may also include a plurality of lowered surfaces 1016. At least two triangular shaped lower surfaces 1016 may be located at the lateral sides of the staples cavities 1014 located in the second row 1022b with an apex of the triangle pointing toward combined raised surfaces 1018 located in adjacent rows 1022a, 1022c of staple cavities 1014. At least one triangular shaped lower surface 1018 located at a lateral side of the third row 1022c distal to the slot 1006 may have an apex of the triangle pointing toward combined raised surface 1018 located in adjacent row 1022b. In addition, staple cartridge 1000 may include a triangular-shaped end lower surface 1016a positioned between a distal end 1001b of the cartridge 1000 and the second row 1022b of staple cavities 1014 with an apex of the aligned with the second row 1022b and pointing toward to the proximal end 1001a of the staple cartridge 1000. As shown, cartridge 1000 may not include any lowered surfaces directly adjacent to staple cavities 1014 of the first row 1022a.

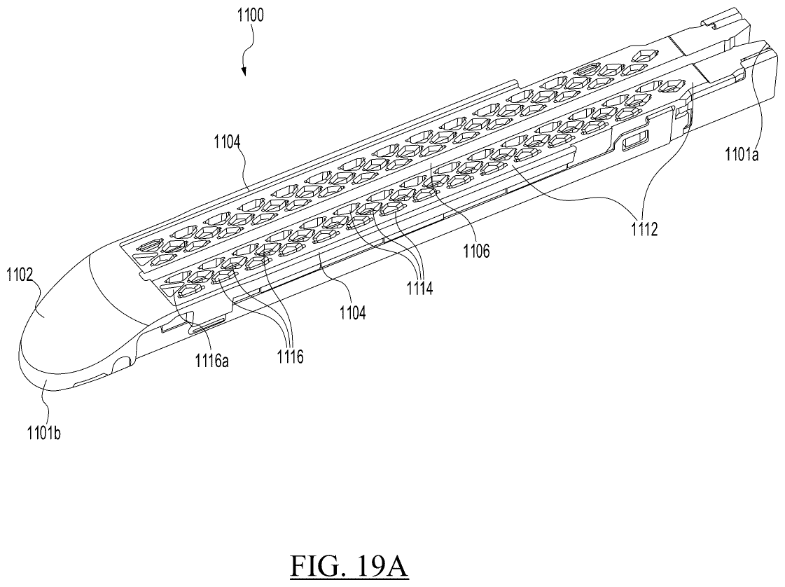

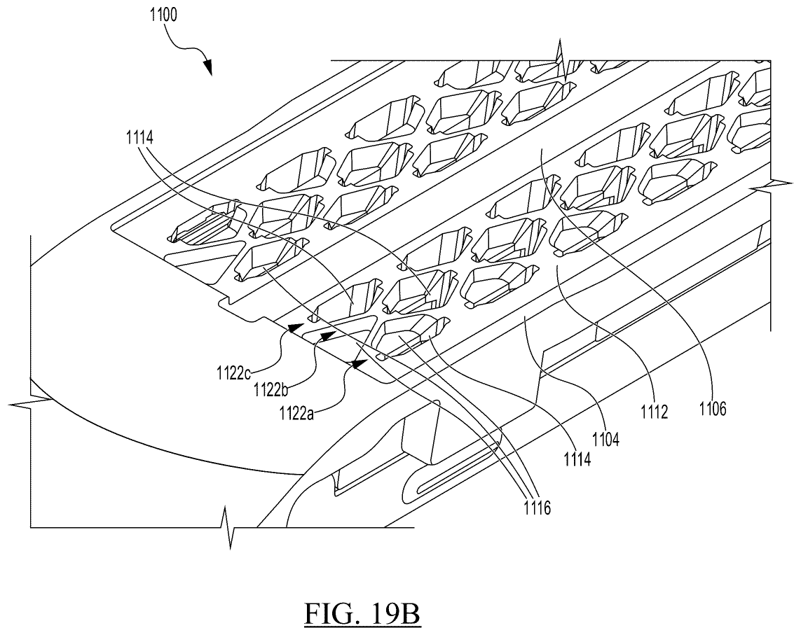

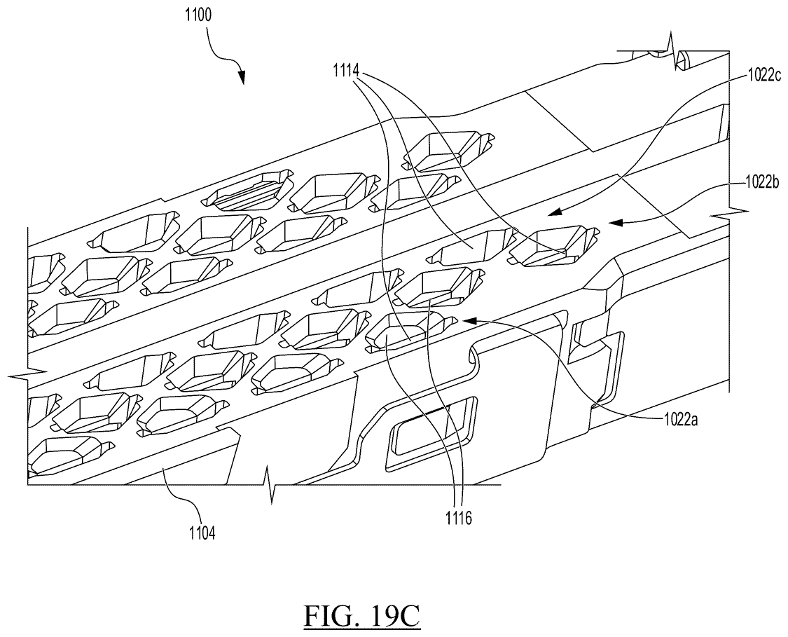

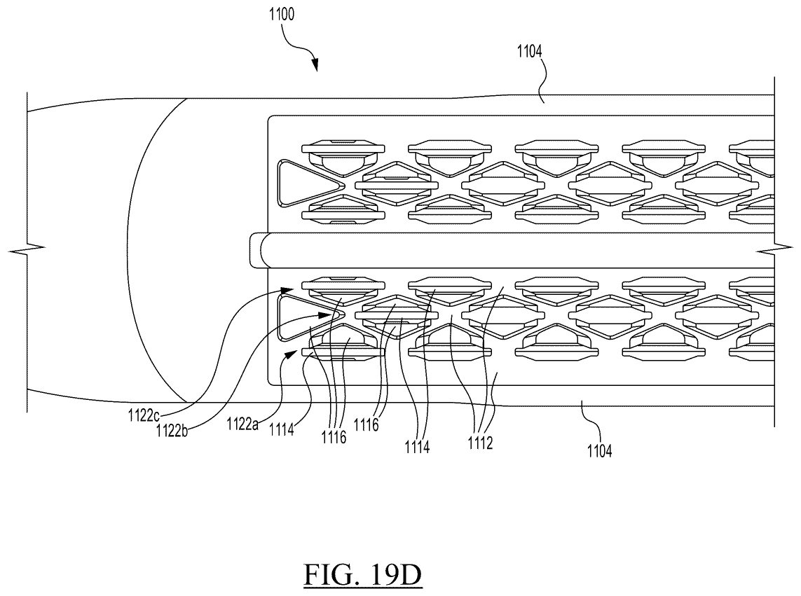

FIGS. 19A-19D illustrate an exemplary staple cartridge 1100 similar to exemplary staple cartridge 1000 of FIGS. 18A-18D, with a few notable differences such as having a flat deck 1112 without raised surfaces adjacent staple cavities 1114. Deck 1112 may include a plurality of lowered surfaces 1116, 1116a positioned and shaped similarly to lowered surfaces 1016, 1016a of exemplary cartridge 1000. In addition, deck 1112 may include additional lowered surfaces 1116 positioned adjacent staple cavities 1114 of the first row 1122a on the side closest to the slot 1106. Thus, each staple cavity 1114 of the first, second, and third rows 1122a, 1122b, 1122c may have at least on adjacent lower surface 1016 that is triangular-shaped.

Both staple cartridges 1000 and 1100 allow for staples 300 having a pre-formed height about 0.179 inches, which is more than previous devices, with a deck height of about 0.060 inches, which is shorter than previous devices.

The surgical adjunct 604 may have one or more of the properties described below to enable the adjunct to be flexible when in vivo, but yet remain in a certain position attached to the cartridge when outside of the body. For example, the polyurethane may serve to create an adjunct 604 that is flexible when going into the body but “sets” to its final mechanical properties as the plasticizer is absorbed in vivo. In some examples, a plasticizer may be added to the polyurethane foam to lower its glass transition temperature to be within the below described ranges as well as conform to the other listed properties. Regardless, an adjunct 604 having one or more of the below properties consistently creates a hemostatic or near hemostatic seal on tissue.

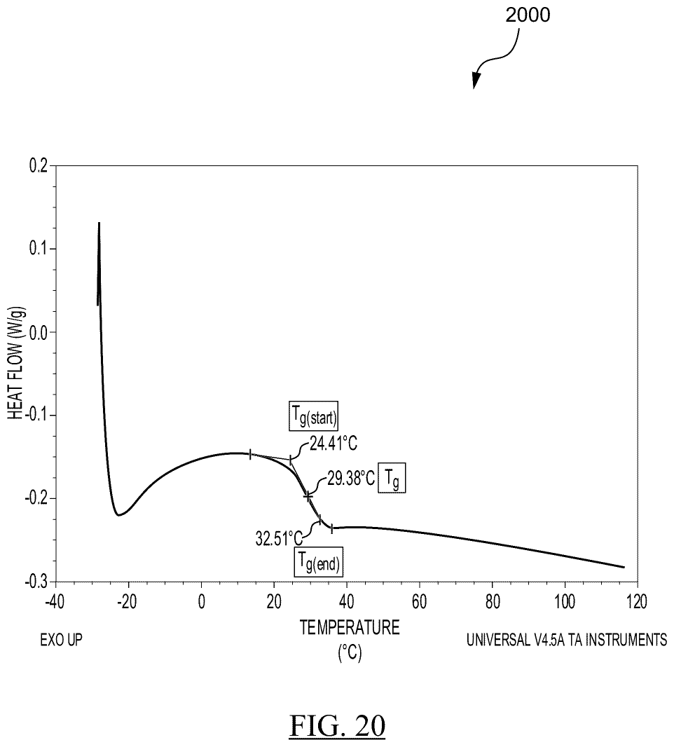

The surgical adjunct 604 may include a polyurethane foam with or without a plasticizer where the glass transition temperature of the surgical adjunct 604 is about 0° C. to about 40° C. (e.g., about 19.4° C.), such as about 7.5° C. to about 22.5° C. or about 12.5° C. to about 17.5° C. The glass transition temperature of the adjunct 604 is obtained by using a standard differential scanning calorimetry (DSC) system. Using the DSC system with its output 2000 shown in FIG. 20, an adjunct 604 was equilibrated at about −40° C., heated at about 40° C./min to about 120° C., held isothermally for about 1 minute, cooled at about 40° C./min to about −40° C., held isothermally for about 1 minute, and then heated at about 10° C./min to about 120° C., where the glass transition temperature T_{g} was measured and recorded by the DSC system.

The surgical adjunct 604 may include a volumetric ratio of the polyurethane foam to the total volume of the adjunct 604 of about 0.125 to about 0.325, such as about 0.175 to about 0.225 or about 0.19 to about 0.21.

The plasticizer may include one or more of a low molecular weight glycol, polyethylene glycol, polyvinylpyrrolidone, dibutyl sebacate, glyceryl triacetate, glyceryl behenate, hexanoic acid, decanoic acid, octadecanoic acid, boric ester, and a fatty acid. In some examples, the plasticizer includes one or more fatty acids.

In some examples, the adjunct 604 includes a polydioxanone (PDO) film disposed on one or more surfaces of the polyurethane foam. In some examples, the PDO film is adhered to at least a bottom or crown side of the adjunct 604. In some examples, the PDO film has a thickness of about 20 μm to about 100 μm, such as about 40 μm.

The adjunct 604 may have a compression strength of about 30 kPa to about 70 kPa, such as about 30 kPa to about 60 kPa (e.g., about 42 kPa), about 30 kPa to about 50 kPa, about 32.5 kPa to about 37.5 kPa. In order to test compression strengths, an adjunct 604 was placed in a humid warm environment at approximately 37° C., compressed to a first height, then a second height shorter than the first height, and then released back to the first height at which point the adjunct's compression strength was measured.

In some examples, the adjunct 604 may have a (peak) tensile strength of about 50 kPa to about 150 kPa or about 30 kPa to about 90 kPa such as about 45 kPa to about 85 kPa or about 55 kPa to about 75 kPa. In some examples, the adjunct 604 will have (peak) tensile strength of about 110 kPa to about 150 kPa. Tensile strength is measured on an adjunct 604 having the dog-bone configuration shown and described with respect to FIGS. 17A and 17B. Specifically, the adjunct's 604 tensile strength is measured after submerging it in water at a temperature of about 37° C. for less than a minute and then running a tensile strength test.

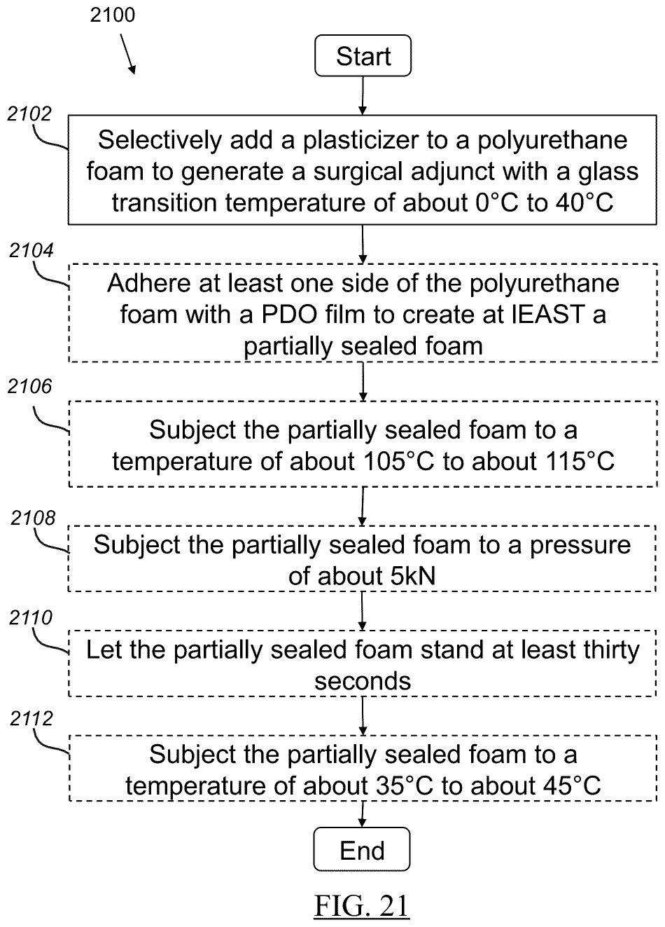

Referring to FIG. 21, a surgical adjunct 604 may be made by using method 2100, which includes selectively (step 2102) adding a plasticizer to a polyurethane foam to generate a surgical adjunct 604 described throughout. The plasticizer may be placed onto or within a surgical adjunct via bulk swelling in solution or via direct deposition to plasticize the polyurethane foam in order to reduce the foams glass transition temperature and/or reduce the mechanical strength of the polyurethane foam. In some examples, the method 2100 may optionally include adhering (optional step 2104) at least one side (e.g., bottom or crown side) of the polyurethane foam with a PDO film to create at least a partially sealed foam, subjecting (optional step 2106) the partially sealed polyurethane foam to a temperature of about 105° C. to about 115° C. for about 24 minutes, subjecting (optional step 2108) the partially seal foam to a pressure of about 5 kN, letting (optional step 2110) the partially sealed foam stand at least thirty seconds (e.g., about 60 seconds), and subjecting (optional step 2112) the partially sealed foam to a temperature of about 35° C. to about 45° C. for about 24 minutes. Step 2108 may be conducted simultaneously with steps 2106, 2110, and 2112. In some examples, surgical adjunct 604 is created without a plasticizer and the polyurethane foam is generated to have certain ratios of air to polyurethane for a given volume due to pore sizes and amounts.

Methods for Embedding Medical Additives into Bioabsorbable Materials

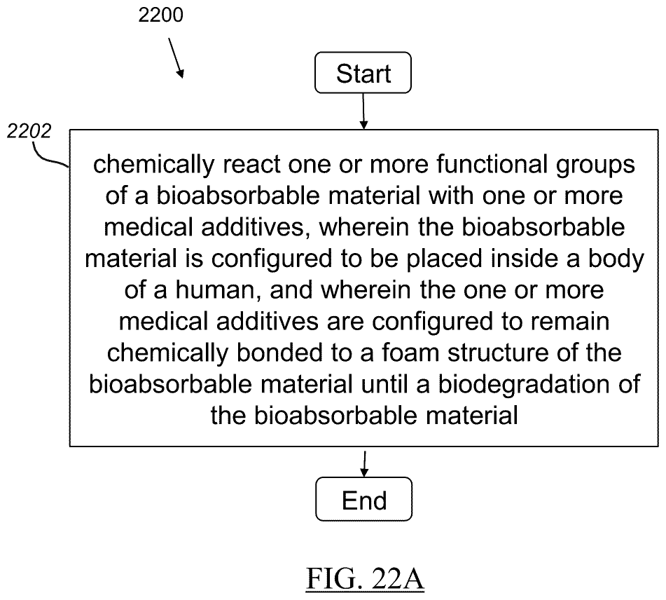

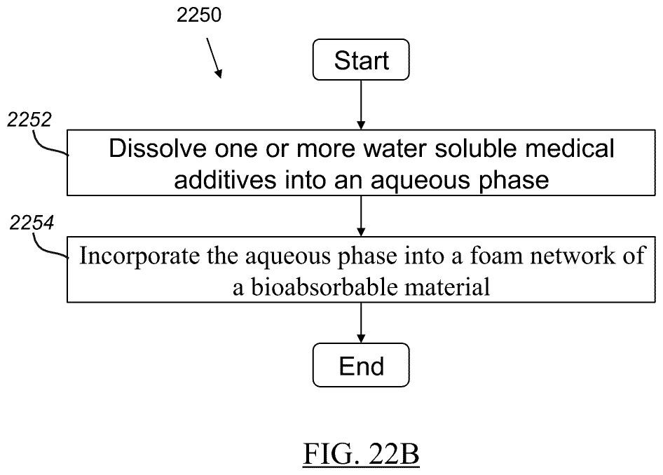

Referring to FIGS. 22A and 22B, a surgical adjunct 604, including a bioabsorbable material that itself includes a foam, may be embedded with one or more medical additives using method 2200 (FIG. 22A) or method 2250 (FIG. 22B). The embedding techniques described herein may offer an added benefit of increasing positive in vivo interactions, including biocompatibility, wound healing, tissue integration, chemotherapy, anti-inflammatory, bone growth and integration, ligament and tendon repair, etc., when the adjunct is delivered to the tissue site, as described herein. As such, the embedding techniques described herein may allow the bioabsorbable material itself to aid in the healing process of the surrounding tissue. In addition, the embedding techniques described herein may offer an added benefit of preventing fibrous encapsulation of the foam cushion, and/or providing tunable release profiles for a variety of medical additives delivered to the tissue site.

Specifically with respect to FIG. 22A, the method 2200 used for embedding medical additives into a bioabsorbable material (e.g., a foam) may include chemically reacting one or more functional groups of the bioabsorbable material with one or more medical additives (step 2202). Chemical embedment may include introducing the medical additive(s) through chemical reaction and bonding with a foam structure of the bioabsorbable material, such that the bioabsorbable material may maintain a slow and steady release profile as it degrades over time.

In some examples, the bioabsorbable material may include a polyurethane and/or be a reaction product of a polyol and an isocyanate. In such examples, the medical additive(s) used may react with the polyol.

In some examples, the functional group(s) may include isocyanate groups. In such examples, the medical additive(s) may include polydrugs (e.g., ibuprofen, acetaminophen, naproxen, etc.), and the method may include endcapping the isocyanate reactive groups in the prepolymer with the polydrug(s) such that the polydrug(s) remain chemically bonded to a foam structure of the bioabsorbable material until biodegradation. The polydrug(s) may be configured to treat pain and/or promote wound healing, tissue growth, infection reduction, localized radiotherapeutics, immunotherapies, and the like.

In some examples, the functional group(s) may include polyol hydroxyl groups (e.g., polyethylene glycol modified (PEGylated) biopharmaceuticals), such as ones that may be incorporated as biobetters. In such examples, the medical additive(s) may include medications that treat pain and/or promote wound healing, tissue growth, infection reduction, and the like. The method may include chemically reacting the polyol hydroxyl groups with the medical additive(s) by functionalizing the polyol hydroxyl groups with the medical additive(s). The method may include chemically reacting the polyol hydroxyl groups with one or more natural materials, such as alginate, hyaluronic acid, etc.

In some examples, chemically reacting the functional groups of the bioabsorbable material may include modifying the functional groups using one or more reactive surfactants with enhanced polyether modifications, such as incorporating PEGylated biopharmaceuticals as biobetters.

Specifically with respect to FIG. 22B, the method 2250 used for embedding medical additives into a bioabsorbable material (e.g., a foam) may include physical embedment of medical additives into the bioabsorbable material, which may involve introducing the medical additives into the bioabsorbable material as independent substances which do not themselves react chemically with the system. As shown, method 2250 may involve dissolving one or more water soluble medical additives into an aqueous phase (step 2252), and incorporating the aqueous phase into a foam network of the bioabsorbable material (step 2254). In some examples, the medical additives may include water soluble drugs and/or vitamins, such as antihypertensive drugs or B-complex vitamins. These drugs and/or vitamins can be incorporated into a foam network of the bioabsorbable material during processing and released during biodegradation of the bioabsorbable material after delivery to the tissue.

In some examples, dissolving the water-soluble medical additives into the aqueous phase may involve generating a colloidal suspension including silver nanoparticles. This process may allow for off-gassing produced by the water, while keeping the silver nanoparticles embedded in the bioabsorbable material. This process may provide an added benefit of enhanced wound healing due to the significant antimicrobial effects of silver nanoparticles.

In some examples, the method may include an emulsion method whereby the medical additives may be encapsulated within “shells” or areas of the bioabsorbable material cells, and released during biodegradation of the bioabsorbable material. For example, the medical additives may be encapsulated within the bioabsorbable material via in-situ hydrogel formation (e.g., entrapment within the bioabsorbable material cells after the adjunct is positioned and exposed to water within the body, etc.).

Surgical Adjuncts with Fluid Barrier to Mitigate Transmural Fluid Ingress

Surgical adjunct 604 may be hydrophobic or include a hydrophobic material to prevent fluid ingress from organs such as the colon from contaminating a patient's body cavity. The polyurethane foam may include hydrophobic properties. For example, the polyurethane foam of the surgical adjunct 604 may include a hydrophobic chemical additive when forming the foam. Some examples of additives may include ceramic nanoparticles (e.g., calcium phosphate), fatty acids (e.g., oleic acid, decanoic acid, hexanoic acid, dodecanoic acid), ionic liquids, enteric coating, a photocurable resin, or other long chain surfactants. Fatty acids may have pH dependent solubility. For example, increasing the chain length from hexanoic to decanoic acid can be used to change the solubility in low pHs with increasing solubility in basic conditions. This may be an additional mechanism to include a transient hydrophobicity whereby the fatty acid is entrapped within the network in the presence of acid. The retention would allow for a barrier to fluid flow. In areas not exposed to acidic condition, the fatty acid can be dissolved over a prescribed period to allow for absorption consistent with the neat adjunct material.

The mechanism for ionic liquids would be based on the potential counterion exchange within the body (sodium exchange) to allow dissolution of the liquids within the body. In the associated state, the ionic liquid can provide increased hydrophobicity. As such, the inclusion of ionic liquids can give rise to a transient hydrophobicity. As such, the inclusion of ionic liquids can give rise to a transient hydrophobicity.

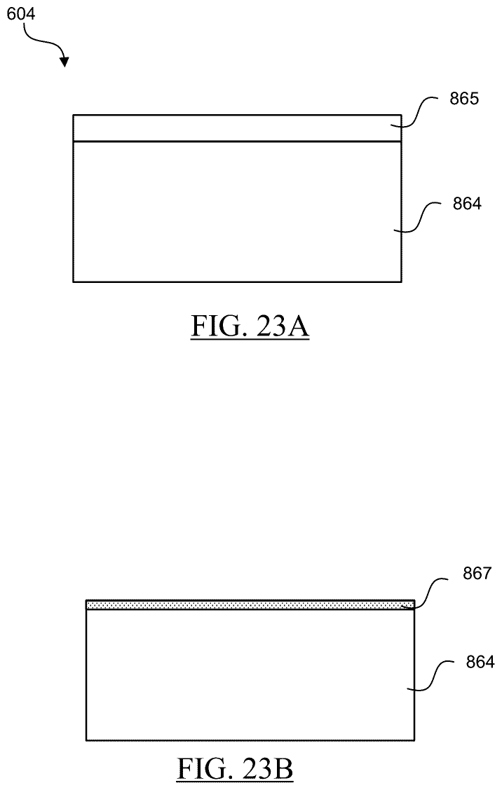

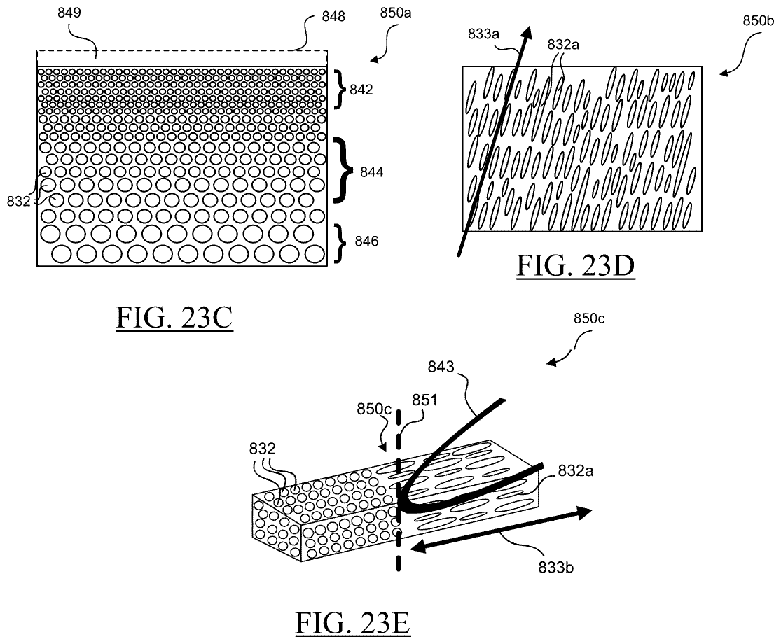

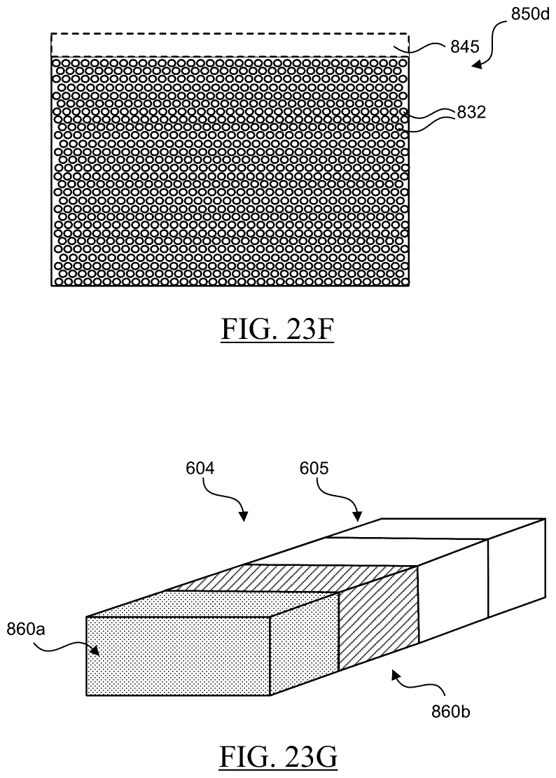

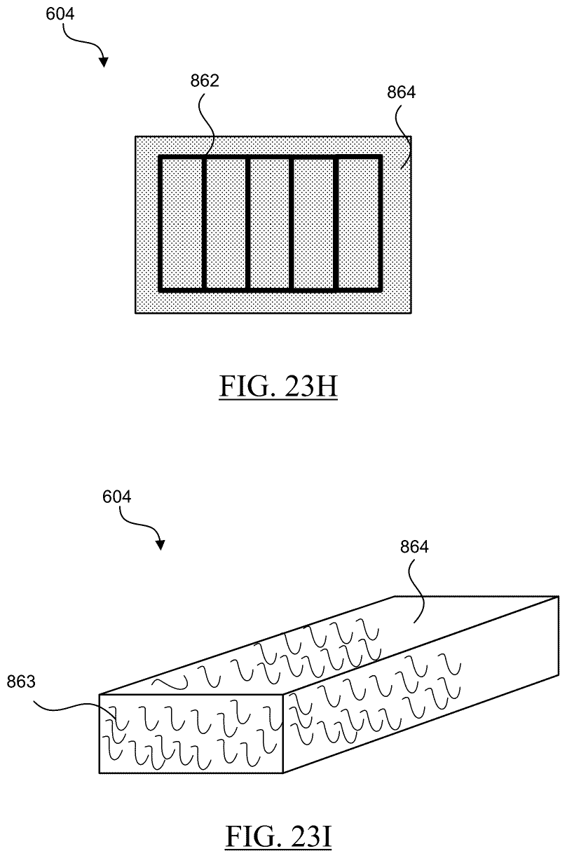

As shown in FIG. 23A, the hydrophobic additive may be a hydrophobic coating 865 disposed on the polyurethane foam. A hydrophobic coating 865 would not alter the chemistry of the polyurethane foam and would be layered on top of the foam partially or fully blocking fluid flow. Although the coating is shown on surface it may cover one or more surfaces or a portion thereof. The coating 865 may include multiple layers of different, similar, or the same material to promote coating adhesion of increase hydrophobicity. In addition, multiple layers may be used to minimize impact on adsorption by alternating surface eroding coatings. For example, a surface eroding polyester (e.g., polypropylene fumarate) may be used as the exterior coating with a final later of an enteric coating. The acid generation during the polyester degradation would create a local acidic environment that would eliminate water permeability while retaining a hydrophobic surface. After the acid environment is dissipated, the enteric coating would rapidly dissolve and allow the adjunct to be absorbed. Examples of coating material include at least one fatty acid (e.g., oleic acid, decanoic acid, hexanoic acid, dodecanoic acid), the at least one long chain surfactant, the at least one enteric coating (e.g., cellulose derivatives or methacrylate copolymers), the at least one photocurable resin, or combinations thereof. The hydrophobic coating may be applied to the polyurethane foam via inkjet printing, direct deposition, thermal spraying, cold dynamic spraying, cold spraying, electro spraying, ultrasonic spray coating, dip coating, screen printing, solution deposition, spin coating, film coating, exterior lamination, and lithography.

The surgical adjunct 604 may include multiple different or similar application methods to coat an inner and other surface of the polyurethane foam. The surgical adjunct 604 may include one or more hydrophobic coatings as well as a hydrophobic chemical additive added to the foam. In some examples, the hydrophobic coating may react with a surface of the polyurethane foam to create hydrophobic properties for the surgical adjunct 604. In some examples, hydrophobic chemical additive and/or the hydrophobic property may include an additional step (e.g., submerging in water, heating, cooling) to activate the hydrophobicity properties.

Furthermore, the hydrophobic coating may be used for time relates therapeutics within the adjunct to assist in healing, reduce inflammation, and treat diseases.

As shown in FIG. 23B, the surgical adjunct 604 may include one or more patterns 867 having creating one or more hydrophobic surfaces or portions thereof. Hydrophobicity may be generated via specific surface texture on the surface of the surgical adjunct 604. Such surface patterns 867 may have one or more specific repeating patterns with pitches smaller than a diameter of a water droplet. For example, the surface pattern may include at least one repeating pattern with a pitch that is less than about 0.005 centimeters. The specific hydrophobic surface patterns 867 may be generated via interferometric lithography, stereolithography, selective ablation, lyophilization, plasma etching, chemical etching, or surface-initiated polymer brushes.

The advantages of adding one or more of the above-described hydrophobicity features is that such surgical adjunct may be able to mitigate and eliminate transmural waning, mitigate transmural risk in vivo, potentially increase retention of the mechanical strength of the surgical adjunct based on physiological conditions, and potentially use the surgical adjunct 604 as an extended-release intra-organ drug delivery mechanism.

Referring to FIG. 23C, the surgical adjunct 604 may include a polyurethane foam with a gradient porosity 850a with pores 832 including small pores 842 disposed adjacent to a first end or first side 848. When placed in a patient's body, the first end 848 may be positioned adjacent to a point of transmural access from a patient's organ inside the patient's body cavity. The pores 832 of polyurethane foam may also include large pores 846 disposed away from the first end 848 and adjunct a second end or second side 849. The polyurethane foam may also include transitional pores 844 that include pores generally arranged such that smaller diameters pores are closer to the first end 848 and larger diameter pores are positioned further away from first end 848. The small pores 842 and some of the transitional pores 844 (e.g., the smaller ones) may prevent the fluid ingress from the patient's organ (e.g., organ from digestive system) into the surgical adjunct the rest of the patient's body while the large pores 846 as well as some of the transitional pores 844 (e.g., some of the larger ones) may allow fluid ingress from other parts of the body such as the blood stream to promote biodegradation of the polyurethane foam. Although pores 832 are described are increasing in size from the first end 848 as they travel away from the first end 848, the pores 832 may increase in size along any direction such as the axial direction 851 (FIG. 23D-23E), lateral direction 833b (FIG. 23D-23E), or both.

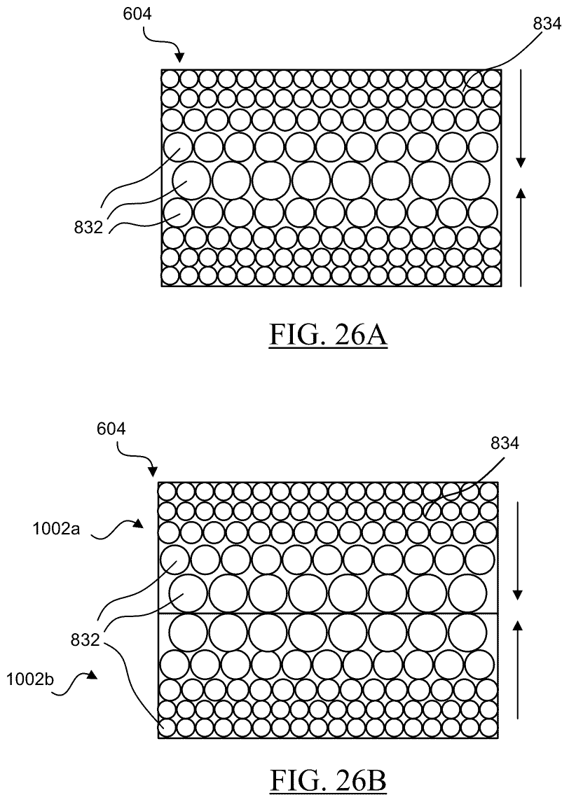

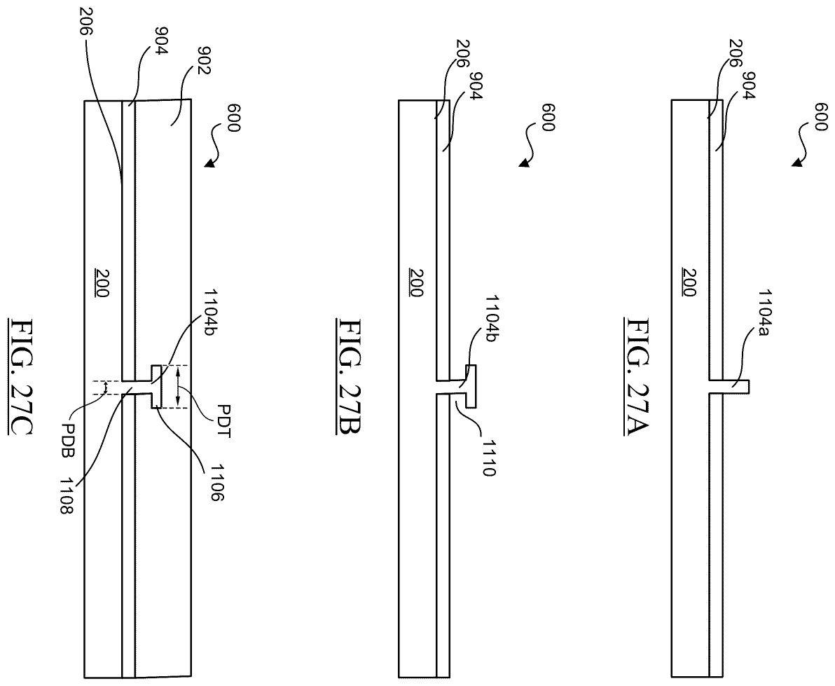

Referring to FIG. 26A, the surgical adjunct 604 may include a double density gradient with respect to its pore diameters. For example, the surgical adjunct 604 may include pores 832 with small diameters (e.g., a range of about 100 μm to about 0.4 mm such as about 0.15 mm to about 0.25 mm) adjacent a first end and a second end that is opposite the first end. Moving from the first end or second end to the center of the surgical adjunct 604 the diameters of the pores 832 continue to increase to a large diameter size (e.g., about 0.4 mm to about 1 mm such as about 0.5 mm to about 0.6 mm). Referring to FIG. 26B, the surgical adjunct 604 may include two bioabsorbable materials 1002a, 1002b, each with a single gradient for pore diameters. These two bioabsorbable materials 1002a, 1002b may be joined together to form a double density gradient. In both FIGS. 26A and 26B, the surgical adjunct may include struts 834 that may increase in thickness near the larger pores than the smaller pores.

Referring to FIGS. 23D and 23E, the surgical adjunct 604 may include a polyurethane foam with directional morphology. For example, the pores 832a of the polyurethane foam may be aligned in a first direction 833a to generally control fluid flow in the first direction 833a and second direction 833a (opposite directional arrow), which is a direction opposite the first direction (see FIG. 23D). As another example, the polyurethane foam may include directional pores 832a aligned in a first direction 833b (also lateral direction) as well as include small pores 832 or closed cells pores (see FIG. 23E). Both the directional pores 832a and the small pores 832 (or closed cell pores) may prevent fluid 843 through certain portions of the polyurethane foam but allow fluid 843 to flow through the portion of the foam that includes the directional pores.

Referring to FIG. 23F, the surgical adjunct 604 may include an impermeable barrier, skin, or moisture barrier 845 disposed on one or more surfaces of the polyurethane foam. The polyurethane foam may include small pores (or closed cells pores) 832.

Referring to FIG. 23G, the surgical adjunct 604 may include a section of impermeable or hydrophobic material 860b or a moisture barrier along with a buttress material (e.g., an adhesive) 860a section and one or more sections of polyurethane foam 605. The hydrophobic material 860b is disposed between the buttress material 860 and the polyurethane foam 605. The moisture barrier may include a film including aliphatic polyesters such as polylactic acid (PLA), polyglycolic acid (PGA), polycaprolactone (PCL), polydioxanone (PDO), poly(trimethylene carbonate) (PTMC), or copolymer combinations.

Referring to FIG. 23H, the surgical adjunct 604 may include matrix 862 of a hydrophobic film or moisture barrier within the polyurethane foam 864. Referring to FIG. 23I, the surgical adjunct 604 may include material 863 interlaced within the polyurethane foam 864. The material 863 may be include a hydrophobic coating or be hydrophobic. The moisture barrier may include a film including aliphatic polyesters such as polylactic acid (PLA), polyglycolic acid (PGA), polycaprolactone (PCL), polydioxanone (PDO), poly(trimethylene carbonate) (PTMC), or copolymer combinations.

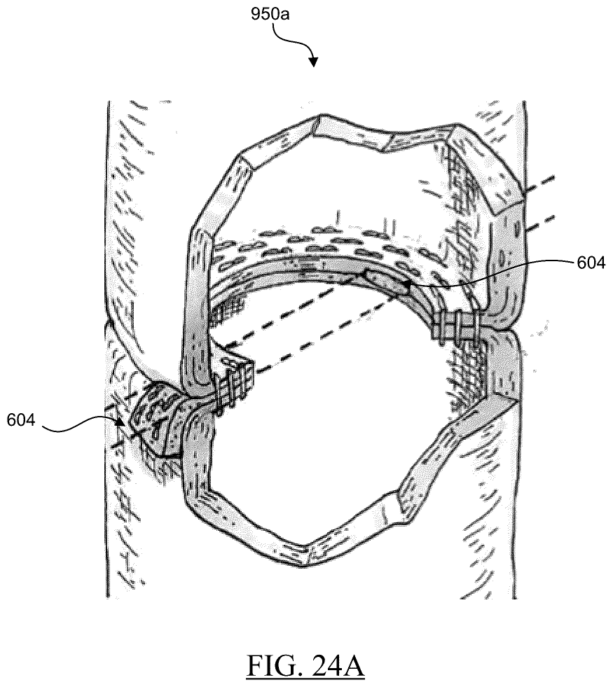

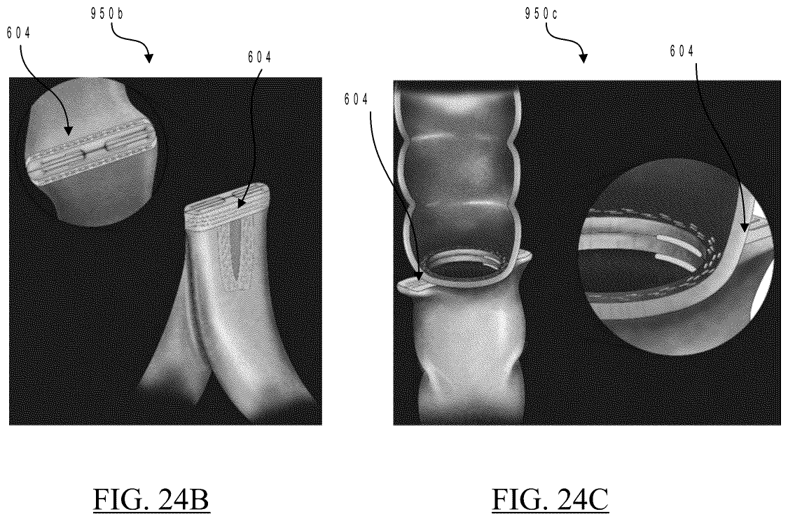

FIGS. 24A-24C are exemplary diagrams 950a, 950b, and 950c showing surgical adjuncts 604 placed transmurally in a patient. For example, biopharmaceuticals such as monoclonal antibodies could be used since it has limited ability to be absorbed within the gastrointestinal tract. The ability to use the surgical adjunct 604 as a carrier for the biopharmaceutical could enable bioavailability within the body without the burden of injections, infusions, etc.

Surgical Adjuncts with Cushion Attachment Adhesive Layers

The use of absorbable stapling adjuncts or other dampening implants (e.g., ligament anchor surgery, tendon repair) requires a balance between the mechanical strength of the implant/adjunct, chemistry (e.g., absorbable), and deployment requirements. The strength to retain staples, sutures, screws, etc., may conflict with the requirements for endoscopic deployment (e.g., compression through trocar). As the implant strength increases, there is an increased force required to compress the implant for insertion. This may exceed the limits of the endoscopic instrument (e.g., surgical stapling and severing device 100) requiring compromises to the implant performance. While this may be mitigated through phase change materials (e.g., water swell, glass to rubber transitions), this further limit applicable chemistries given the need to have an absorbable implant or adjunct.

A facile method for improving the strength requires to retain stapes, sutures, screws, etc., without compromising the bulk mechanical properties of the implant is the inclusion of a backing material or film. Given the potential need for increase mechanical strength, the backing material may be a different material from the surgical adjunct or implant. The backing material may be incorporated in situ or adhered to the surgical adjunct or implant.

An example of the adherence during processing may include melt welding commonly used to bond thermoplastic materials. In this process, a combination of temperature, pressure, and time are controlled to have diffusion of the thermoplastics into each other. This process can be used to a lesser extent between a thermoplastic and thermoset material where the thermoplastic material flows around a geometric feature on the thermoset material and a mechanical bond is formed.

The stapling adjuncts or damping implants will likely be composed of either a thermoplastic or thermoset material. In the case of a thermoplastic material, there are limitations in retaining the desired shape with the thermal processing as the material may flow during processing. Conversely, the incorporation of a backing material with a thermoset is limited during thermal processing as the material will not flow and form an intimate bond interface.

Another limiting case for melt welding in the case of absorbable materials (e.g., a polyurethane foam) is the potential degradation during processing. Many absorbable materials, particularly faster absorbing materials, are thermally labile and thus may lose mechanical properties, generate degradant species, etc. during processing.

As such, there are limitations associated with (i) utilizing thermally labile materials, (ii) an inability to spatially modulate adhesive properties, and (iii) adherence limited to mechanical encapsulation for thermoset/thermoplastic combinations. Thus, there are inefficiencies for backing material incorporation a surgical adjunct or damping implant. Therefore, alternative methods and materials are discussed herein.

Additionally, films used to attach a surgical adjunct to a staple cartridge deck may be mechanically attached to the cartridge deck along with the surgical adjunct to prevent early release of the surgical adjunct from a staple cartridge deck.

Additionally, the surgical adjunct itself can be designed of made to include a gradient or double gradient of pore diameters in order to help maintain both compressive and tensile properties needed to create a hemostatic seal and also strong enough to allow a surgeon to grasp and manipulate the tails of the cushion. Such a morphology would mimic that of bone or other materials found in nature.

In some examples, the adjunct 604 includes a polydioxanone (PDO) film disposed on one or more surfaces of the polyurethane foam. In some examples, the PDO film is adhered to at least a bottom or crown side of the adjunct 604. In some examples, the PDO film has a thickness of about 20 μm to about 100 μm, such as about 40 μm.

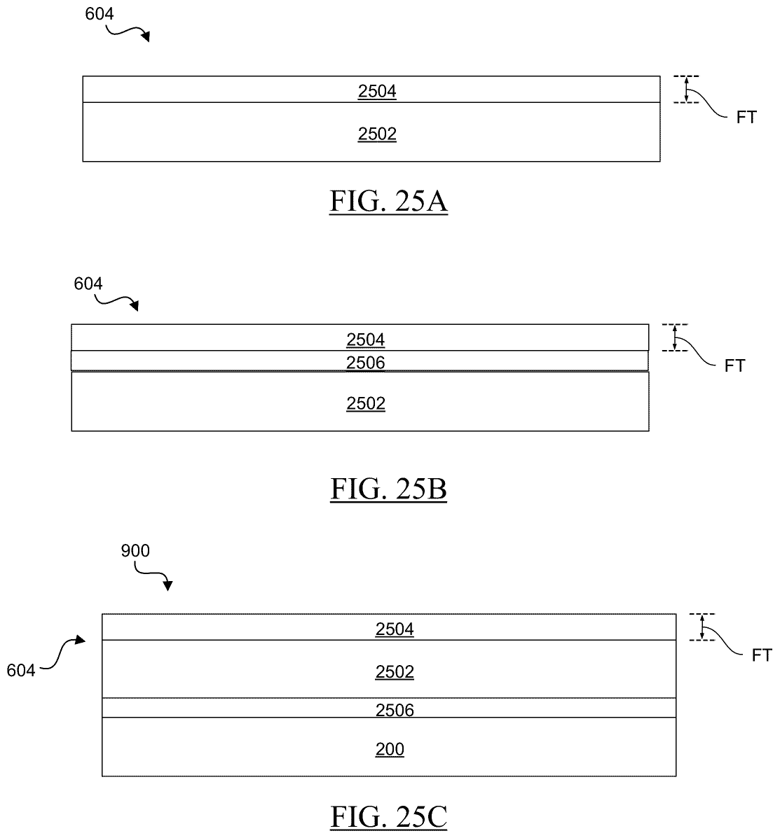

Referring to FIG. 25A, the surgical adjunct 604 may include a bioabsorbable material 2502 (e.g., bioabsorbable foam such a polyurethane foam) and a film 2504 having a film thickness FT disposed on the absorbable material. This film thickness FT of the film 2504 may range from about 0.0003 inches to about 0.010 inches. For example, the film may be a skin made of the same material as the bioabsorbable materials 2502 and may include a film thickness FT with a range of about 0.001 inches to about 0.010 inches. In other instances, the film 2504 can be a mostly continuous surface on the staple crown side of the bioabsorbable materials 2502 to help guide the staples 300 when firing and may have a film thickness ranges from about 0.00039 inches to about 0.0039 inches (e.g., about 0.00046 inches to about 0.020 inches). This film 2504 may include pores with diameters in the range of about of about 0.0005 inches to about 0.005 inches when formed from the same material (e.g., polyurethane or polyurethane foam) as the bioabsorbable material 2502. In some examples, when the film 2504 comprises the same bioabsorbable material (e.g., polyurethane) the film 2504 may have a higher density than then bioabsorbable material 2502. When the film 2504 includes other materials, the film thickness FT may range from about 0.001 inches to about 0.003 inches. For example, the film 2504 may include polydioxanone (PDO), poly(delta-gluconolactone) (PGL-1), poly(glycolide/l-lactide) (PGL-2), polyglycolic acid (PGA), a glycolide and epsilon caprolactone copolymer (PGCL), a glycolide and l-lactide copolymer, urethane, polycaprolactone (PCL), polyglactin 370 (PG-370), polyglactin 185 (PG-185), or combinations thereof.

Referring to FIG. 25B, the surgical adjunct 604 may include a bioabsorbable material 2502, and a film 2504 disposed on the bioabsorbable material 2502 with an adhesive 2506 disposed between the film 2504 and the bioabsorbable material 2502. Referring to FIG. 25C, the surgical adjunct 604 may include a bioabsorbable material 2502 disposed on a staple cartridge 200 with an adhesive 2506 disposed between the bioabsorbable material 2502 and the cartridge 200. Additionally, a film 2504 is disposed on a surface of the bioabsorbable material 2502. In some examples, the adhesive may include polyvinylpyrrolidone. In some examples, the adhesive 2506 is bioabsorbable.

Referring to FIG. 26A, the surgical adjunct 604 may include a double gradient with respect to its pore diameters. For example, the surgical adjunct 604 may include pores 832 with small diameters (e.g., a range of about 100 μm to about 0.4 mm such as about 0.15 mm to about 0.25 mm) adjacent a first end and a second end that is opposite the first end. Moving from the first end or second end to the center of the surgical adjunct 604 the diameters of the pores 832 continue to increase to a large diameter size (e.g., about 0.4 mm to about 1 mm such as about 0.5 mm to about 0.6 mm). Referring to FIG. 26B, the surgical adjunct 604 may include two bioabsorbable materials 1002a, 1002b, each with a single gradient for pore diameters. These two bioabsorbable materials 1002a, 1002b may be joined together to form a double gradient. In both FIGS. 26A and 26B, the surgical adjunct may include struts 834 that may increase in thickness near the larger pores than the smaller pores.

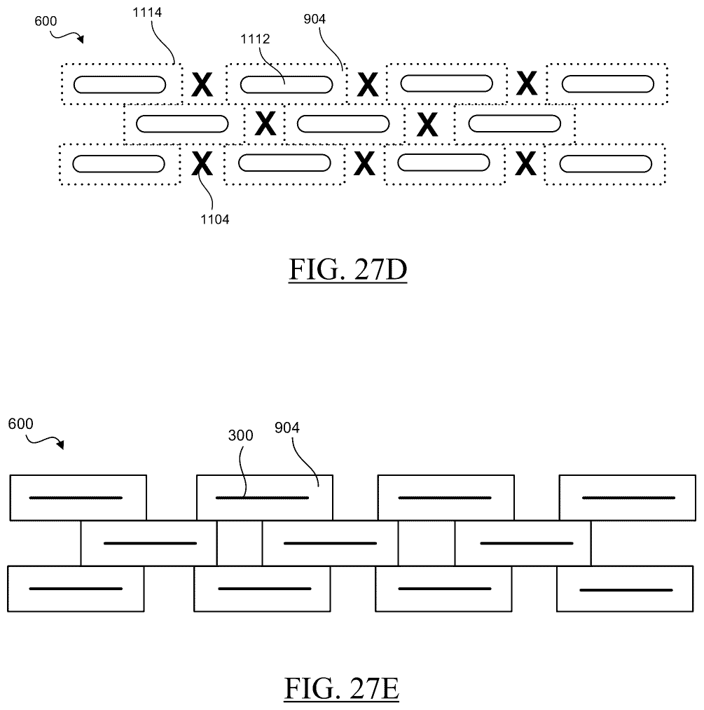

Referring to FIG. 27A, stapling assembly 600 may include a cartridge 200 that includes a deck 206 and at least one post 1104a extending away from that deck. The stapling assembly includes a film 904 disposed on deck 206. The at least one post 1104a may be heated to form a post 1104b with a top 1106 and a bottom 1108 as shown in FIG. 27B. As shown in FIG. 27C, the bioabsorbable material 902 may be disposed on the film 904 and within a gap 1110 between the top 1006 of the at least one post 1104b and the film 904. Since the top 1106 may have a diameter PDT that is greater than a diameter PDB of the bottom 1108, the bioabsorbable material 902 may be mechanically locked in place or attached to the stapling assembly 600.

FIG. 27D shows a top view of an exemplary stabling assembly 600. As shown, the posts 1104 may be disposed between staple slots 1112 of the cartridge 200. In addition, the film 904 may include perforations 1114 at least partially surrounding or covering the staple slots 1112 and the posts 1104 to allow the film to support the staples 300 shown in FIG. 27E.

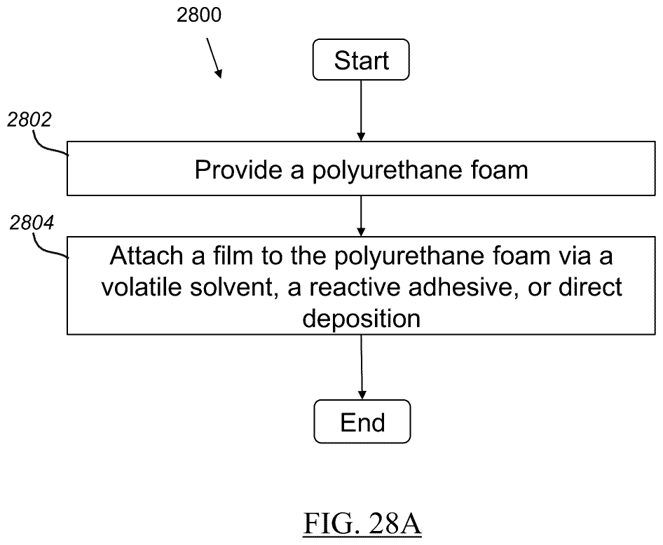

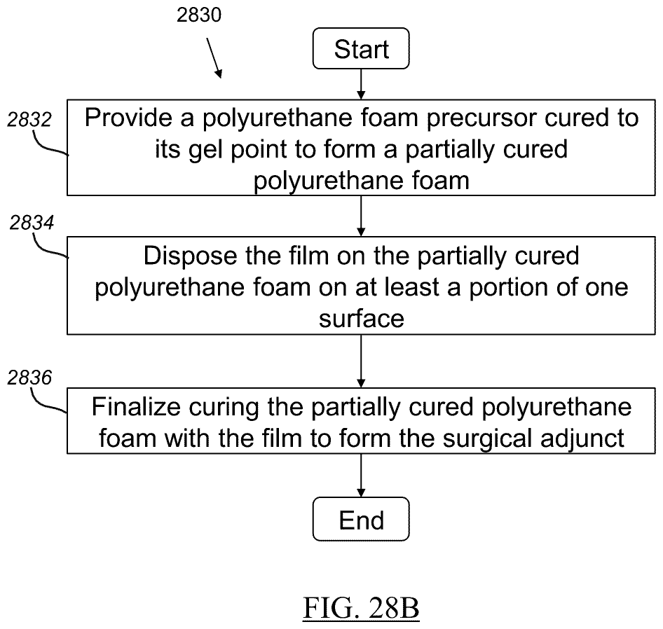

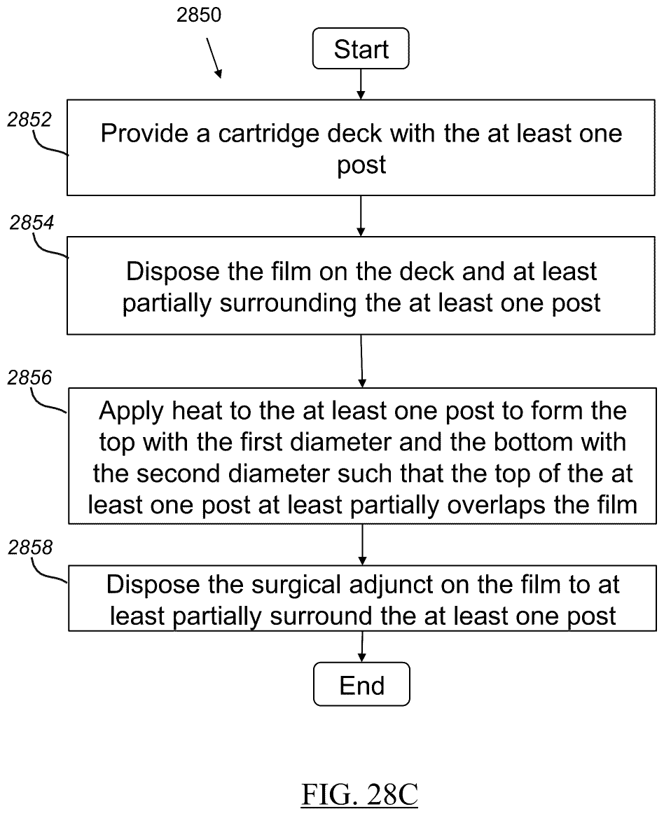

Referring to FIG. 28A, method 2800 relates to adding a film to a bioabsorbable material such as a polyurethane foam. The method includes providing 2802 a polyurethane foam and attaching 2804 a film to the polyurethane foam via a volatile solvent (e.g., solvent welding), a reactive adhesive, or direct deposition.

Solvent welding may be used to add a film to bioabsorbable material. For example, solvents such as ethyl acetate, dichloromethane, acetone may be used. Using this process eliminated the need for thermal processing and eliminated the need for spatial arrangement of bonding parts with the film.

Reactive adhesives may be used to add a film to the bioabsorbable material. Some reactive adhesives may include polyurethanes, epoxies, acrylates, etc. The reactive adhesive may be directly deposited onto the adjunct and/or the film. This can be patterned with both surface area and mass modulate the adhesive strength along and across the bioabsorbable material. Such an application may be useful in the case where selective release of the film is designed to minimize the overall bioabsorbable material (e.g., film only were contacted with staples with the remaining staying with the endoscopic instrument. In addition to the application process, the chemistry may be used to tune the adhesive properties. The reactive adhesive may be applied using inkjet printing, direct deposition, thermal spraying, cold dynamic spraying, cold spraying, electro spraying, ultrasonic spray coating, dip coating, screen printing, and spin coating. Using adhesives is beneficial because it is possible to bond two thermoset materials (film and bioabsorbable material) where material flow is not required. Using reactive adhesives may eliminate the need for thermal processing, eliminate need to arrange bonding points between film and bioabsorbable material, and allows for the modulating of adhesive forces across the surgical adjunct to create a selective release feature if needed.

Direct deposition may be used to directly apply a film to the bioabsorbable material. The film itself may be distributed in a solution or be composed of a reactive mixture that is subsequently applied to the bioabsorbable material. Direct depositions method for adding the film to the bioabsorbable material may include stereolithography, lithography, holographic printing, inject printing, direction deposition, thermal spraying, cold dynamic spraying, cold spraying, electro spraying, ultrasonic spray coating, dip coating, screen printing, and spin coating. Reactive mixtures for these processes, may include polyurethane, epoxy. Photocurable formulations may be used as well. These can include a photoinitiator, a solvent, inhibitors, photocurable oligomer or monomer, light absorber, and mixtures thereof. The advantages of directly applying the film include the ability to conformally apply the film, spatial control of the film, being able to easily apply the film, and flexibility in applying the film in terms of rate, thickness, etc. for different uses.

Referring to FIG. 28B, method 2830 relates to adding or forming a film when forming the polyurethane foam or in-situ. The method 2830 includes providing 2832 a polyurethane foam precursor cured to its gel point to form a partially cured polyurethane foam 902, disposing or forming 2834 the film 904 on the partially cured polyurethane foam on at least a portion of the one surface, finalize curing 2836 the partially cured polyurethane foam with the film to form the surgical adjunct 604.

When using thermoset materials (e.g., polyurethane foams) for surgical adjuncts, there is a potential to incorporate the material during the adjunct fabrication process. Before the completion of curing and/or specific stochiometric imbalance, the film may be applied to the adjunct. For best results, the addition of the film should be completed after the gel point of the thermoset material to ensure that the film does not constrain the adjunct formation. After applying the film, the curing process should resume to allow for chemical bonds between the thermoset material and the film. This will especially be the case where free hydroxyl groups are present in the case of polyurethane-based adjuncts. The chemical bonds would provide strength profiles that may exceed any adhesive interaction between the film and the thermoset material. Advantages to using this method include, elimination of thermal processing, potential covalent attachment between the film and the thermoset material, and the elimination of the need for a separate adhesive.

Additionally, the film such as PDO may be overmolded on the bioabsorbable material.

Referring to FIG. 28C, method 2850 of attaching a film 904 and bioabsorbable material 902 to a cartridge deck is illustrated. Method 2850 may include providing 2852 a cartridge deck 206 with the at least one post 1104, disposing 2854 the film on the deck and at least partially surrounding the at least one post 1104, applying 2856 heat to the at least one post 1104 to form the top 1106 with the first diameter and the bottom 1108 with the second diameter such that the top 1106 of the at least one post 1104 at least partially overlaps the film 904. Method 2850 also includes disposing 2858 the surgical adjunct on the film 904 to at least partially surrounds the at least one post 1104.

### Cushion Attachment Adhesives and Cartridge Surface Arrangements

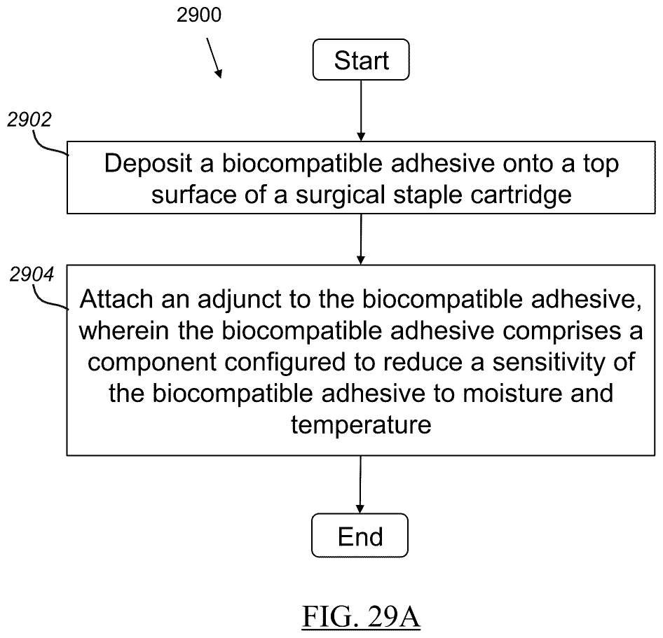

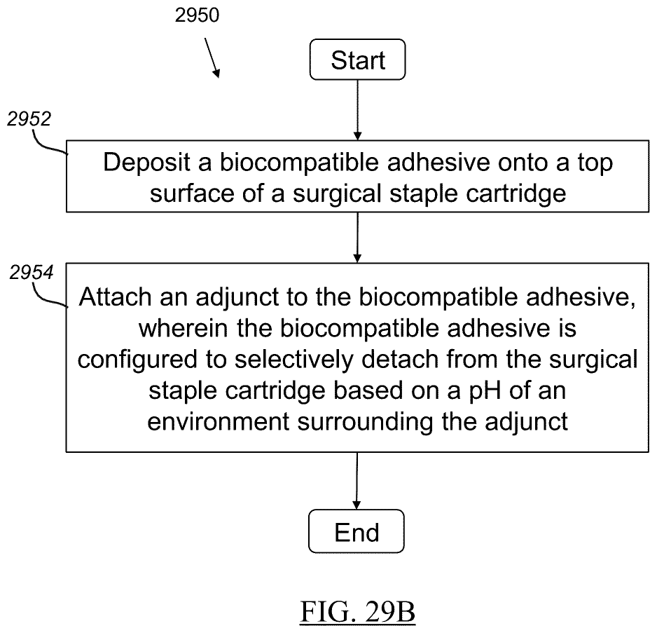

Referring to FIGS. 29A and 29B, an adjunct (e.g., 604) may be attached to a surgical staple cartridge (e.g., 200) using a biocompatible adhesive. For stapling adjunct delivery, as discussed herein, the use of biocompatible adhesives may help to ensure the adjunct remains on the endoscopic instrument during trocar insertion and potential tissue manipulation. Current methods of attachment, such as via the use of pressure sensitive adhesives, typically result in challenges, such as low adhesive or cohesive strength, potential need to reseat the adhesive (e.g., reduced adhesive strength as a function of shelf-life), and/or sensitivities to moisture or temperature fluctuations. Accordingly, the attachment methods described herein may help to achieve a balance between the force required to keep the adjunct on the instrument, and to release the adjunct during stapling or other surgical procedure. The attachment methods described herein may provide for a balance between material choice, surface coverage, and/or adjunct cohesive strength. For example, the cohesive strength of the adjunct may be higher than the adhesive force of either the instrument/adhesive or adhesive/adjunct interface, or the cohesive strength of the adhesive.

Specifically with respect to FIG. 29A, the method 2900 used for attaching an adjunct to a surgical staple cartridge using a biocompatible adhesive may include depositing a biocompatible adhesive 216 onto a top surface 206 of a surgical staple cartridge 200 (step 2902), as particularly shown in FIG. 2B, and attaching an adjunct 604 to the biocompatible adhesive 216 (step 2904), as particularly shown in FIG. 6A.

In some examples, the biocompatible adhesive may include a component configured to reduce the sensitivity of the biocompatible adhesive to moisture and temperature. For example, the biocompatible adhesive may be sensitive to temperatures above approximately 40 degrees Celsius and/or relative humidities greater than 60%. The component may be a hydrophobic component, such as a high melting wax or a reactive adhesive (e.g., polyurethane).

A high melting wax may help to mitigate a temperature sensitivity of the biocompatible adhesive. The wax may have a melting temperature of at least approximately 60 degrees Celsius. In addition, the wax may be hydrophobic, which may minimize moisture sensitivity of the biocompatible adhesive. The wax may include, for example, candelilla wax, microcrystalline wax, montan wax, and/or white wax. The wax may have a sharp viscosity transition (e.g., solid to liquid transition within 5 degrees Celsius) as a function of temperature.

Various applications may be employed for using the wax to attach an adjunct to a surgical staple cartridge, such as dip coating, electrospray, ultrasonic spray coating, inkjet, direct deposition, screen printing, spin coating, etc. These application technologies can be conducted via either batch processes or in situ whereby a continuous application process can be used on either an extrusion or web-based material handling operation. The conformal nature of these techniques can be controlled via wax and substrate temperature control, deposition rate, substrate motion/rate, etc. Additionally, these application techniques can provide spatial resolution on the adjunct and/or endoscopic instrument, which may allow for differing adhesive properties based on differing surface area coverage and/or mass (e.g., balance adhesive and wax cohesive strength for failure location optimization, etc.), as well as precision to avoid critical endoscopic instrument (e.g., instrument 100) features (e.g., staple pockets, etc.).

The use of a high melting wax may provide an added benefit of reduced sensitivity to temperature, such as during shipment or storage, reduced sensitivity to moisture, such as during a surgical procedure, and ease of application during manufacturing.

A reactive adhesive, such as polyurethane, epoxy, acrylate, etc., can similarly be used to reduce moisture and/or temperature sensitivities of the biocompatible adhesive. This may be accomplished through specific adhesive patterning and/or surface area minimization. The reactive nature of the adhesive may allow the adhesive to be applied at low viscosities (e.g., greater than or equal to approximately 5 centipoise (cP)) that may increase the fidelity of the surface application, which can help to minimize the amount of material, coverage area (e.g., placement on a low surface area to minimize interference with other endoscopic instrument features), and/or equipment complexity. Various applications may be employed for using a reactive adhesive to attach an adjunct to a surgical staple cartridge, such as electrospinning, electrospray, dip coating, thermal spray, screen printing, direction deposition, etc.

The use of a reactive adhesive may provide an added benefit of minimal material usage, lower surface area coverage, and ease of application during manufacturing.

In some examples, the biocompatible adhesive may include a component configured to reduce a sensitivity of the biocompatible adhesive to moisture and temperature. The use of a hydrogel adhesive may allow for a balance of adhesive forces before and during a surgical procedure. In the package, as well as during manufacturing, the hydrogel may be in a dehydrated state that may have significant adhesive and cohesive strength. The primary mode of adhesive strength is specific chemical interactions with the stapling adjunct and endoscopic instrument (e.g., hydrogen bonding, dipole-dipole, etc.). These interactions may be significantly higher (e.g., up to approximately 20 kJ/mol) than a pressure sensitive adhesive given the significant hydrophilic nature of the hydrogel. Additionally, the dehydrated state can be tuned to have significant cohesive strength as well through crosslinker concentration and specific intramolecular hydrogen bonding. Once the hydrogel is rehydrated during the surgical procedure, both the cohesive and adhesive strength can be reduced allowing for release of the stapling adjunct from the endoscopic instrument.

Another feature of hydrogels is the utilization of either covalent and/or ionic crosslinks. Most synthetic hydrogels may include a covalently crosslinked network with irreversible bonds. Another class of hydrogels utilizes ionic crosslinks which are reversible and ion dependent. These hydrogels have multivalent ions that act as crosslinks centers to increase the mechanical strength of the hydrogel. In the presence of a more electronegative ion, counterion exchange may occur. The switching between a multivalent and monovalent ion (e.g., calcium to sodium ion, etc.) can cause a reduction in mechanical strength to the point of dissolution in an aqueous environment. This has the potential advantage of quick dissolution in the surgical environment as the multivalent counterion is exchanged with sodium. In this fashion, there are two mechanisms for release in the surgical environment: water swelling due to moisture environment, and counterion exchange to cause dissolution of the hydrogel. Additionally, the use of ionic crosslinks may allow for the use of naturally occurring materials, such as carrageenan, alginate, polysaccharide-based, cellulose derivatives, etc., that may increase biocompatibility of the overall system.

In the case where the hydrogel is designed to remain attached to the stapling adjunct after the procedure, it can be used to deliver therapeutic agents (e.g., active pharmaceutical ingredients (APIs), growth factors, etc.). These could be designed for either extended or short-term therapies through different microencapsulation technologies or solubility characteristics of the agent. Additionally, the hydrogel can be used to house living cells. The inclusion of a bio ink for cell viability could facilitate the loading of the cells either before or after the stapling adjunct is opened in the surgical suite.

Overall, the hydrogel could be composed of a naturally occurring material or a photocurable network. The photocurable formulation can include a photoinitiator, solvent, inhibitors, photocurable oligomer or monomer, light absorber, or mixtures thereof.

Various applications may be employed for using a hydrogel network to attach an adjunct to a surgical staple cartridge, such as stereolithography/lithography, holographic printing, inkjet printing, direct deposition, thermal spraying, cold dynamic spraying, cold spraying, electro spraying, ultrasonic spray coating, dip coating, screen printing, spin coating, etc.

The use of a hydrogel may provide an added benefit of tailored strength, such as during shipment, packaging, or a surgical procedure, utilization of naturally sourced materials, potential to deliver therapeutic agents, reduced temperature sensitivity, utilization of lower cohesive strength stapling adjuncts, and ease of application during manufacturing.

Specifically with respect to FIG. 29B, the method 2950 used for attaching an adjunct to a surgical staple cartridge using a biocompatible adhesive may include depositing a biocompatible adhesive onto a top surface of a surgical staple cartridge (step 2952), and attaching an adjunct to the biocompatible adhesive (step 2954). The biocompatible adhesive may include a component configured to selectively detach from the surgical staple cartridge based on a pH of an environment surrounding the adjunct.

In some examples, the biocompatible adhesive may include an enteric adhesive. Enteric adhesives may be used for selective detachment as a function of pH. For example, in an acidic environment (e.g., an environment having a pH of less than approximately 6.0), the adhesive would not allow water ingress and would retain its adhesive strength. Once in the presence of an environment approaching a neutral pH (e.g., an environment having a pH of greater than approximately 6.0), the adhesive would detach due to water ingress. This may be used in cases where gastric fluid needs to be bypassed with the stapling adjunct adhered. Once reaching another cavity or place within the gastrointestinal tract, the stapling adjunct would release at lower detachment forces. Potential materials are, but not limited to, cellulose derivatives, methacrylate copolymers, etc. The enteric adhesives may be applied by one or more of the application techniques described herein.

In some examples, the biocompatible adhesive may include a stimuli responsive material. The utilization of stimuli responsive materials both to in vivo and externally applied stimuli may eliminate the balance between adhesive strength and cohesive strength of the stapling adjunct, thus increasing the range of adjunct mechanical properties that can be used. The in vivo stimuli may be pH or counter ion exchange, as further discussed herein. The transition from the acidic trigonal form to the basic tetrahedral form can be observed through ultraviolet (UV) absorption and thus quantified for various arylboronic acid, styrylpyrene, o-nitrobenzyl, coumarin, and polyol combinations. As the dissociation is based on binding constants, multiple layer systems can be designed to deploy within a set range of pH, or to respond based on changes in the local environment. In this manner, the adhesive could be designed to have specific behavior (e.g., mechanical strength) before and after surgical implantation.

The stimuli responsive material may be applied externally, such as via light mediation, such that bonds are configurable based on exposure to differing wavelengths of light (e.g., between approximately 300 to 475 nanometers (nm)). Activation of these systems for adhesive control could be conducted using a transmittable wavelength through the tissue or an endoscopic procedure for activation, such as photoreversible cycloaddition of styrylpyrene. These molecular changes can be tailored to allow a macroscopic geometric change.

Stimuli responsive materials may also be used to design an architecture transformable polymer via dynamic covalent chemistries that can be split between redox-, photo-, and mechano-responsive chemistries. Sufficient molecular mobility may allow the transition from linear to complex architectures resulting in dimensional changes within a system. The stimuli responsive adhesives may be applied by one or more of the application techniques described herein.

The use of a stimuli responsive adhesive may provide an added benefit of maintaining full mechanical strength for a predetermined timeframe (e.g., induction period per external excitation), mitigating moisture and thermal sensitivities, potential inclusion of a controlled API, and balance of adhesive and stapling adjunct cohesive properties.

As will be appreciated by one skilled in the art, the embodiments described above are cited by way of example, and the present invention is not limited by what has been particularly shown and described hereinabove. Rather, the scope of the invention includes both combinations and sub combinations of the various features described hereinabove, as well as variations and modifications thereof which would occur to persons skilled in the art upon reading the foregoing description and which are not disclosed in the prior art.

### List of Clauses

In some examples, disclosed devices (e.g., end effector, surgical adjunct, and/or staple cartridges) and methods involving one or more disclosed devices may involve one or more of the following clauses:

Clause A1: A bioabsorbable material configured to be delivered to tissue, the material comprising: a shape-memory polymer compressible in a delivery configuration and configured to swell within a predetermined period of time; wherein the shape-memory polymer comprises one or more functional groups for reversible bonding between adjacent functional groups to transition between an approximately linear polymer and an approximately non-linear polymer upon exposure to a stimulation.

Clause A2: The bioabsorbable material of clause A1, wherein the shape-memory polymer transitions from the delivery configuration to a swollen configuration when exposed to a temperature in a range of about 34° C. to about 40° C.

Clause A3: The bioabsorbable material of clause A1, wherein the stimulation comprises at least one of heat, light, water, electrical, magnetic, electromagnetic, ultrasound, and pH.

Clause A4: The bioabsorbable material of clause A1, wherein the shape-memory polymer comprises a reaction product of a polyol and an isocyanate.

Clause A5: The bioabsorbable material of clause A1, wherein the functional groups comprise a diene and a dienophile moieties, and wherein the shape-memory polymer is configured to reversibly transition upon exposure to a temperature in a range of about 34° C. to about 40° C.

Clause A6: The bioabsorbable material of clause A1, wherein the functional groups comprise a styrylpyrene moiety, and wherein the shape-memory polymer is configured to reversibly transition upon exposure to a wavelength of light ranging from about 310 nm to about 450 nm.

Clause A7: The bioabsorbable material of clause A1, wherein the functional groups comprise an azo moiety, and wherein the shape-memory polymer is configured to reversibly transition upon exposure to a temperature above about 60° C.

Clause A8: The bioabsorbable material of clause A1, wherein the functional groups comprise an ortho-nitrobenzyl moiety, and wherein the shape-memory polymer is configured to reversibly transition upon exposure to a wavelength of light ranging from about 310 nm to about 440 nm.

Clause A9: The bioabsorbable material of clause A1, wherein the functional groups comprise a coumarin moiety, wherein the shape-memory polymer is configured to reversibly transition to a first compression strength upon exposure to a wavelength of light ranging from about 200 nm to about 260 nm; and wherein the shape-memory polymer is configured to reversibly transition to a second compression strength upon exposure to a wavelength of light ranging from about 350 nm to about 560 nm.

Clause A10: The bioabsorbable material of clause A1, wherein the functional groups comprise an anthracene moiety, and wherein the shape-memory polymer is configured to reversibly transition to the approximately non-linear configuration upon exposure to a wavelength of light greater than about 300 nm, and transition to the approximately linear configuration upon exposure to a wavelength of light less than about 300 nm.

Clause A11: The bioabsorbable material of clause A5, wherein the functional groups comprise at least one of a disulfide moiety and a diselenide moiety, and wherein the shape-memory polymer is configured to reversibly transition upon exposure to at least one of a change in temperature, a change in pH, a reactive oxygen species, or combinations thereof.

Clause A12: The bioabsorbable material of clause A1, wherein the predetermined period of time ranges from approximately 0.01 seconds to approximately 120 seconds.

Clause A13: The bioabsorbable material of clause A2, wherein the bioabsorbable material has a compression strength of about 50 kPa to about 90 kPa in the delivery configuration, and a compression strength of about 30 kPa to about 70 kPa in the swollen configuration.

Clause A14: The bioabsorbable material of clause A1, wherein the bioabsorbable material degrades according to a degradation profile in response to exposure to a fluid comprising at least one of a predetermined temperature, an enzyme-catalyst, and a predetermined pH.

Clause A15: The bioabsorbable material of clause A1, further comprising one or more medical additives configured to remain chemically bonded to the shape-memory polymer.

Clause A16: The bioabsorbable material of clause A15, wherein the one or more medical additives are further configured to be released to or approximate the tissue.

Clause A17: A bioabsorbable material configured to be delivered to tissue, the material comprising: a shape-memory polymer compressible in a delivery configuration and configured to swell within a predetermined period of time upon exposure to a stimulation, the shape-memory polymer comprising a polyurethane backbone and one or more functional groups for reversible bonding between adjacent functional groups to transition between an approximately linear polymer to an approximately non-linear polymer.

Clause A18: The bioabsorbable material of clause A17, wherein the stimulation comprises at least one of heat, light, water, electrical, magnetic, electromagnetic, ultrasound, and pH.

Clause A19: The bioabsorbable material of clause A17, wherein the predetermined period of time ranges from approximately 0.01 seconds to approximately 120 seconds.

Clause A20: The bioabsorbable material of clause A17, wherein the one or more functional groups comprise at least one of a diene and a dienophile moiety, a styrylpyrene moiety, an azo moiety, an ortho-nitrobenzyl moiety, a coumarin moiety, an anthracene moiety, a disulfide moiety, a diselenide moiety, or combinations thereof.

Clause A21: A method to form a bioabsorbable material configured to be placed inside a body of a human, the method comprising the steps of: adding, to a polyurethane polymer, a functional group comprising at least one of a diene and a dienophile moiety, a styrylpyrene moiety, an azo moiety, an ortho-nitrobenzyl moiety, a coumarin moiety, an anthracene moiety, a disulfide moiety, a diselenide moiety, or combinations thereof, and chemically bonding the polyurethane polymer and functional groups to form a shape-memory polymer.

Clause A22: The method of clause A21, further comprising the step of: exposing the shape-memory polymer to a stimulation comprising at least one of heat, light, water, electrical, magnetic, electromagnetic, ultrasound, and pH.

Clause A23: The method of clause A22, wherein the shape-memory polymer is configured to reversibly transition between an approximately linear polymer to an approximately non-linear polymer.

Clause A24: The method of clause A22, wherein the shape-memory polymer is compressible in a delivery configuration.

Clause A25: The method of clause A21, wherein the bioabsorbable material comprises a delivery height ranging from about 0.01 mm to about 1 mm when in the delivery configuration.

Clause A26: The method of clause A25, further comprising the step of: increasing a height of the shape-memory polymer to greater than the delivery height.

Clause A27: The method of clause A21, further comprising the step of: exposing the bioabsorbable material to a fluid comprising at least one of a predetermined temperature, an enzyme-catalyst, and a predetermined pH such that the bioabsorbable material degrades according to a degradation profile.

Clause A28: A bioabsorbable material configured to be delivered to tissue, the material comprising: a shape-memory polymer compressible in a delivery configuration and configured to swell within a predetermined period of time; wherein the shape-memory polymer comprises one or more functional groups for reversible bonding between adjacent functional groups to transition between an approximately linear polymer and an approximately non-linear polymer upon exposure to a stimulation.

Clause A29: The material of clause A28, wherein the shape-memory polymer transitions from the delivery configuration to a swollen configuration when exposed to a temperature in a range of about 34° C. to about 40° C.

Clause A30: The material of clauses A28 or A29, wherein the stimulation comprises at least one of heat, light, water, electrical, magnetic, electromagnetic, ultrasound, and pH.

Clause A31: The material of any of clauses A28-A30, wherein the shape-memory polymer comprises a reaction product of a polyol and an isocyanate.

Clause A32: The material of any of clauses A28-A31, wherein the functional groups comprise a diene moiety and a dienophile moiety, and wherein the shape-memory polymer is configured to reversibly transition upon exposure to a in a range of about 34° C. to about 40° C.

Clause A33: The material of any of clauses A28-A31, wherein the functional groups comprise a styrylpyrene moiety, and wherein the shape-memory polymer is configured to reversibly transition upon exposure to a wavelength of light ranging from about 310 nm to about 450 nm.

Clause A34: The material of any of clauses A28-A31, wherein the functional groups comprise an azo moiety, and wherein the shape-memory polymer is configured to reversibly transition upon exposure to a temperature above about 60° C.

Clause A35: The material of any of clauses A28-A31, wherein the functional groups comprise an ortho-nitrobenzyl moiety, and wherein the shape-memory polymer is configured to reversibly transition upon exposure to a wavelength of light ranging from about 310 nm to about 440 nm.

Clause A36: The material of any of clauses A28-A31, wherein the functional groups comprise a coumarin moiety, wherein the shape-memory polymer is configured to reversibly transition to a first compression strength upon exposure to a wavelength of light ranging from about 200 nm to about 260 nm; and wherein the shape-memory polymer is configured to reversibly transition to a second compression strength upon exposure to a wavelength of light ranging from about 350 nm to about 560 nm.

Clause A37: The material of any of clauses A28-A31, wherein the functional groups comprise an anthracene moiety, and wherein the shape-memory polymer is configured to reversibly transition to the approximately non-linear configuration upon exposure to a wavelength of light greater than about 300 nm, and transition to the approximately linear configuration upon exposure to a wavelength of light less than about 300 nm.

Clause A38: The material of any of clauses A28-A31, wherein the functional groups comprise at least one of a disulfide moiety and a diselenide moiety, and wherein the shape-memory polymer is configured to reversibly transition upon exposure to at least one of a change in temperature, a change in pH, a reactive oxygen species, or combinations thereof.

Clause A39: The material of any of clauses A28-A38, wherein the predetermined period of time ranges from approximately 0.028 seconds to approximately 120 seconds.

Clause A40: The material of clauses A28 or A29, wherein the bioabsorbable material has a compression strength of about 50 kPa to about 90 kPa in the delivery configuration, and a compression strength of about 30 kPa to about 70 kPa in the swollen configuration.

Clause A41: The material any of clauses A28-A40, wherein the bioabsorbable material degrades according to a degradation profile in response to exposure to a fluid comprising at least one of a predetermined temperature, an enzyme-catalyst, and a predetermined pH.

Clause A42: The material of any of clauses A28-A41, further comprising one or more medical additives configured to remain chemically bonded to the shape-memory polymer, wherein the one or more medical additives are further configured to be released to or approximate the tissue.

Clause B1: A bioabsorbable material configured to be delivered to tissue, the material comprising: a first polymerizable compound comprising a reaction product of a polyol and an isocyanate and configured to degrade according to a first degradation profile, and a second polymerizable compound configured to degrade according to a second degradation profile different than the first degradation profile; wherein the material undergoes a phase transition in response to exposure to a fluid comprising at least one of a predetermined temperature, an enzyme-catalyst, and a predetermined pH.

Clause B2: The material of clause B1, wherein the first and second polymerizable compounds together form a copolymer backbone.

Clause B3: The material of clause B2, wherein the second polymerizable compound comprises a polyester, a poloxamer, a polypropylene oxide, a polyvinyl pyrrolidone, a poly(l-lactic acid), a poly(lactic-co-glycolic acid), or combinations thereof.

Clause B4: The material of clause B2, wherein a volumetric ratio of the second polymerizable compound to first polymerizable compound is in a range of about 0.125 to about 0.325.

Clause B5: The material of clause B1, wherein the material undergoes phase transition from a swollen state to a collapsed state when exposed to a temperature above the predetermined temperature.

Clause B6: The material of clause B5, wherein the first polymerizable compound and the second polymerizable compound degrade according to the respective first and second degradation profiles when the fluid comprises at least one of a temperature above the predetermined temperature and a pH above the predetermined pH.

Clause B7: The material of clause B1, wherein the predetermined temperature of the phase transition ranges from about 25° C. to about 35° C.

Clause B8: The material of clause B1, wherein the second polymerizable compound forms a coating about the first polymerizable compound such that the coating prevents the first polymerizable compound from exposure to a fluid ingress for a predetermined period of time.

Clause B9: The material of clause B8, wherein the second polymerizable compound comprises a polymethacrylate, a polydioxanone, a polyanhydride, a hydroxypropyl methylcellulose acetate succinate, a cellulose acetate phthalate, a cellulose acetate trimellitate, a hydroxypropyl methylcellulose phthalate, a polyvinyl acetate phthalate, a poly(trimethylene carbonate), a poly(beta-thioether ester ketal), a polypropylene fumarate, a poly(ester urea), a poly(ester amide), or combinations thereof.

Clause B10: The material of clause B8, wherein the predetermined period of time ranges from approximately 10 minutes to approximately 6 weeks.

Clause B11: The material of clause B8, wherein the coating comprises a thickness of about 20 μm to about 100 μm.

Clause B12: The material of clause B8, wherein the predetermined pH ranges from about 6.5 to about 7.4.

Clause B13: The material of clause B1, wherein the material has a compression strength of about 20 to about 70 kPa.

Clause B14: The material of clause B1, further comprising one or more medical additives configured to remain chemically bonded to at least one of the first polymerizable compound or the second polymerizable compound.

Clause B15: The material of clause B14, wherein the one or more medical additives are further configured to be released into the tissue over a predetermined period of time during at least one of the first degradation profile and the second degradation profile.

Clause B16: A bioabsorbable material configured to be delivered to tissue, the material comprising: a copolymer backbone comprising: a first polymerizable compound comprising a reaction product of a polyol and an isocyanate and configured to degrade according to a first degradation profile, and a second polymerizable compound configured to degrade according to a second degradation profile different than the first degradation profile; wherein the material undergoes a phase transition in response to exposure to the fluid comprising a predetermined temperature.

Clause B17: The material of clause B16, wherein the first polymerizable compound and the second polymerizable compound degrade according to the respective first and second degradation profiles when the fluid comprises a temperature above the predetermined temperature.

Clause B18: A bioabsorbable material configured to be delivered to tissue, the material comprising: a first polymerizable compound comprising a reaction product of a polyol and an isocyanate and configured to degrade according to a first degradation profile, and a second polymerizable compound coating the first polymerizable such that the coating prevents the first polymerizable compound from exposure to a fluid ingress for a predetermined period of time; wherein the material undergoes a phase transition in response to exposure to a fluid comprising a predetermined pH.

Clause B19: The material of clause B18, wherein the predetermined period of time ranges from approximately 10 minutes to approximately 6 weeks and the predetermined pH ranges from about 6.5 to about 7.4.

Clause B20: The material of clause 18, wherein the coating comprises a thickness of about 20 μm to about 100 μm.

Clause B21: A bioabsorbable material configured to be delivered to tissue, the material comprising: a first polymerizable compound comprising a reaction product of a polyol and an isocyanate and configured to degrade according to a first degradation profile, and a second polymerizable compound configured to degrade according to a second degradation profile different than the first degradation profile; wherein the material undergoes a phase transition in response to exposure to a fluid comprising at least one of a predetermined temperature, an enzyme-catalyst, and a predetermined pH.

Clause B22: The material of clause B21, wherein the first and second polymerizable compounds together form a copolymer backbone.

Clause B23: The material of clauses B21-B22, wherein the second polymerizable compound comprises a polyester, a poloxamer, a polypropylene oxide, a polyvinyl pyrrolidone, a poly(l-lactic acid), a poly(lactic-co-glycolic acid), or combinations thereof.

Clause B24: The material of any of clauses B21-B23, wherein a volumetric ratio of the second polymerizable compound to first polymerizable compound is in a range of about 0.125 to about 0.325.

Clause B25: The material of any of clauses B21-B24, wherein the material undergoes phase transition from a swollen state to a collapsed state when exposed to a temperature above the predetermined temperature.

Clause B26: The material of any of clauses B21-B25, wherein the first polymerizable compound and the second polymerizable compound degrade according to the respective first and second degradation profiles when the fluid comprises one or more of a temperature above the predetermined temperature and a pH above the predetermined pH.

Clause B27: The material of any of clauses B21-B26, wherein the predetermined temperature of the phase transition ranges from about 25° C. to about 35° C.

Clause B28: The material of clause B21, wherein the second polymerizable compound forms a coating about the first polymerizable compound such that the coating prevents the first polymerizable compound from exposure to a fluid ingress for a predetermined period of time.

Clause B29: The material of clause B28, wherein the second polymerizable compound comprises a polymethacrylate, a polydioxanone, a polyanhydride, a hydroxypropyl methylcellulose acetate succinate, a cellulose acetate phthalate, a cellulose acetate trimellitate, a hydroxypropyl methylcellulose phthalate, a polyvinyl acetate phthalate, a poly(trimethylene carbonate), a poly(beta-thioether ester ketal), a polypropylene fumarate, a poly(ester urea), a poly(ester amide), or combinations thereof.

Clause B30: The material of any of clauses B28-B29, wherein the predetermined period of time ranges from approximately 10 minutes to approximately 6 weeks.

Clause B31: The material of any of clauses B28-B30, wherein the second polymerizable compound comprises a thickness of about 20 μm to about 100 μm.

Clause B32: The material of any of clauses B28-B31 wherein the predetermined pH ranges from about 6.5 to about 7.4.

Clause B33: The material of any of clauses B21-B32, wherein the material has a compression strength of about 20 to about 70 kPa.

Clause B34: The material of any of clauses B21-B33, further comprising one or more medical additives configured to remain chemically bonded to one or both of the first polymerizable compound and the second polymerizable compound.

Clause B35: The material of any of claims B21-B34, wherein the one or more medical additives are further configured to be released into the tissue over a predetermined period of time during one or both of the first degradation profile and the second degradation profile.

Clause C1: A bioabsorbable material configured to be delivered to tissue, the material comprising: a porous body comprising at least one polymer comprising: a first zone comprising a first crosslink density; and a second zone comprising a second crosslink density different than the first zone, wherein the first and second zones form a gradient of compression strength along a portion of the porous body.

Clause C2: The material of clause C1, wherein the porous body comprises polyurethane and at least one of the first zone and the second zone comprises a polymerizable compound comprising crosslinkable units, the polymerizable compound configured to react with the polyurethane and undergo crosslinking between crosslinking units upon exposure to a stimulation.

Clause C3: The material of clause C2, wherein the stimulation comprises at least one of heat, light, ultrasound waves, and pH.

Clause C4: The material of clause C2, wherein the polymerizable compound comprises at least one of a chain extender, a reactive monomer, a photoinitiator and a stimuli-responsive functional group; and wherein at least one of the chain extender, reactive monomer, photoinitiator and stimuli-responsive functional group are configured to modulate an amount of crosslinking between crosslinking units.

Clause C5: The material of clause C4, wherein the chain extender comprises fumaric acid, succinic acid, maleic acid, or combinations thereof.

Clause C6: The material of clause C2, wherein the polymerizable compound comprises an acrylate, a methacrylate, an arylboronic acid, a styrylpyrene, a styrene, a vinyl acetate, an acrylonitrile, a vinylidene dichloride, an isoprene, a butadiene, a chloroprene, or combinations thereof.

Clause C7: The material of clause C2, wherein the polymerizable compound is further configured to degrade to form a degradation product, and wherein the degradation product functions as a medicament when released at or approximate the tissue.

Clause C8: The material of clause C1, wherein the second zone is positioned approximately central along a longitudinal axis of the porous body (LP).

Clause C9: The material of clause C1, wherein the second zone is positioned at approximately opposing ends of the porous body.

Clause C10: The material of clause C1, wherein the second zone is coated over at least one side of the first zone.

Clause C11: The material of clause C10, wherein the coating comprises a thickness of about 20 μm to about 100 μm.

Clause C12: The material of clause C1, wherein the foam has a compression strength of about 30 to about 70 kPa in the first zone, and a compression strength of about 15 to about 50 kPa in the second zone.

Clause C13: The material of clause C1, wherein the second zone comprises a lower crosslinking density than the first zone.

Clause C14: A method to form a bioabsorbable material, the method comprising the steps of: chemically reacting a polyol and an isocyanate to form a porous body; and adding a polymerizable compound to at least a portion of the porous body, wherein the polymerizable compound comprises crosslinkable units, the polymerizable compound configured to undergo crosslinking between the crosslinking units upon exposure to a stimulation and modulate a crosslinking density in the porous body.

Clause C15: The method of clause C14, wherein the porous body comprises polyurethane.

Clause C16: The method of clause C14, wherein the polymerizable compound comprises at least one of a chain extender, a reactive monomer, a photoinitiator and a stimuli-responsive functional group, wherein at least one of the chain extender, reactive monomer, photoinitiator and stimuli-responsive functional group are configured to modulate an amount of crosslinking between the crosslinking units upon exposure to the stimulation.

Clause C17: The method of clause C14, further comprising the step of applying the polymerizable compound approximately centrally along a longitudinal axis of the porous body.

Clause C18: The method of clause C14, further comprising the step of applying the polymerizable compound at approximately opposing ends of the porous body.

Clause C19: The method of clause C14, further comprising the step of: exposing the porous body and the polymerizable compound to at least one of heat, light, ultrasound waves, and pH.

Clause C20: The method of clause C14, further comprising the step of: adding a medical additive to the porous body, wherein the one or more medical additives are further configured to chemically bond with the polymerizable compound and be released at or approximate the tissue over a predetermined period of time.

Clause C21: A bioabsorbable material configured to be delivered to tissue, the material comprising: a porous body comprising at least one polymer comprising: a first zone comprising a first crosslink density; and a second zone comprising a second crosslink density different than the first zone, wherein the first and second zones form a gradient of compression strength along a portion of the porous body.

Clause C22: The material of clause C21, wherein the porous body comprises polyurethane and at least one of the first zone and the second zone comprises a polymerizable compound comprising crosslinkable units, the polymerizable compound configured to react with the polyurethane and undergo crosslinking between crosslinking units upon exposure to a stimulation.

Clause C23: The material of clauses C21-C22, wherein the stimulation comprises at least one of heat, light, ultrasound waves, and pH.

Clause C24: The material of any of clauses C22-C23, wherein the polymerizable compound comprises at least one of a chain extender, a reactive monomer, a photoinitiator and a stimuli-responsive functional group; and wherein at least one of the chain extender, reactive monomer, photoinitiator and stimuli-responsive functional group are configured to modulate an amount of crosslinking between crosslinking units.

Clause C25: The material of clause C24, wherein the chain extender comprises fumaric acid, succinic acid, maleic acid, or combinations thereof.

Clause C26: The material of any of clauses C22-C25, wherein the polymerizable compound comprises an acrylate, a methacrylate, an arylboronic acid, a styrylpyrene, a styrene, a vinyl acetate, an acrylonitrile, a vinylidene dichloride, an isoprene, a butadiene, a chloroprene, or combinations thereof.

Clause C27: The material of any of clauses C22-C26, wherein the polymerizable compound is further configured to degrade to form a degradation product, and wherein the degradation product functions as a medicament when released at or approximate the tissue.

Clause C28: The material of any of clauses C21-C27, wherein the second zone is positioned approximately central along a longitudinal axis of the porous body.

Clause C29: The material of any of clauses C21-C27, wherein the second zone is positioned at approximately opposing ends of the porous body.

Clause C30: The material of any of clauses C21-C27, wherein the second zone is coated over at least a portion of one side of the first zone.

Clause C31: The material of any of clauses C28-C30, wherein the second zone comprises a thickness of about 20 μm to about 100 μm.

Clause C32: The material of any of clauses C21-C31, wherein the material comprises a compression strength of about 30 to about 70 kPa in the first zone, and a compression strength of about 15 to about 50 kPa in the second zone.

Clause C33: The material of any of clauses C21-C32, wherein the second zone comprises a lower crosslink density than the first zone.

Clause C34: The material of any of clauses C21-C33, further comprising one or more medical additives configured to remain chemically bonded with the polymerizable compound in at least one of the first zone or the second zone.

Clause C35: The material of clause C34, wherein the one or more medical additives are further configured to be released at or approximate the tissue over a predetermined period of time.

Clause D1: A surgical adjunct 604, comprising: a polyurethane foam comprising a volumetric ratio of the polyurethane foam to the total volume of the surgical adjunct 604 is in a range of about 0.125 to about 0.325; wherein a glass transition temperature of the surgical adjunct 604 is about 0° C. to about 40° C.

Clause D2: The surgical adjunct 604 of clause D1, wherein the volumetric ratio of the polyurethane foam to total volume of the surgical adjunct is about 0.175 to about 0.225.

Clause D3: The surgical adjunct 604 of clause D1, wherein the volumetric ratio of the polyurethane foam to total volume of the surgical adjunct is about 0.19 to about 0.21.

Clause D4: The surgical adjunct 604 of clause D1, wherein the glass transition temperature of the surgical adjunct is about 7.5° C. to about 22.5° C.

Clause D5: The surgical adjunct 604 of clause D1, wherein the glass transition temperature of the surgical adjunct 604 is about 12.5° C. to about 17.5° C.

Clause D6: The surgical adjunct 604 of clause D1, wherein a median pore size of the surgical adjunct 604 is about 0.025 to about 0.300 mm³.

Clause D7: The surgical adjunct 604 of clause D1, further comprising a polydioxanone (PDO) film disposed on at least one surface of the polyurethane foam.

Clause D8: The surgical adjunct 604 of clause D7, wherein the PDO film has a thickness of about 20 μm to about 100 μm.

Clause D9: The surgical adjunct 604 of clause D1, further comprising a plasticizer added to the polyurethane foam.

Clause D10: The surgical adjunct 604 of clause D9, wherein the plasticizer comprises at least one of a low molecular weight glycol, polyethylene glycol, polyvinylpyrrolidone, dibutyl sebacate, glyceryl triacetate, glyceryl behenate, hexanoic acid, decanoic acid, octadecanoic acid, boric ester, and a fatty acid.

Clause D11: The surgical adjunct 604 of clause D9, wherein the plasticizer comprises at least one fatty acid.

Clause D12: The surgical adjunct 604 of clause D1, wherein the surgical adjunct 604 has a compression strength of about 30 kPa to about 70 kPa.

Clause D13: The surgical adjunct 604 of clause D12, wherein the compression strength is about 30 to about 60 kPa.

Clause D14: The surgical adjunct 604 of clause D1, wherein the surgical adjunct 604 has a peak tensile strength of about 50 to about 150 kPa.

Clause D15: The surgical adjunct 604 of clause D1, wherein the surgical adjunct 604 has:
a length of about 40 mm to about 80 mm; a width of about 8 mm to about 12 mm; and a height of about 2.5 mm to about 3.5 mm.

Clause D16: The surgical adjunct 604 of clause D15, wherein the surgical adjunct 604 has a distal end chamfer 604b and a proximal end chamfer 604c, wherein the distal end chamfer comprises a vertical portion extending from a bottom of the surgical adjunct 604, the vertical portion has a height of about 0.009 inches to about 0.029 inches, wherein the distal end chamfer comprises an angled portion extending from the vertical portion to a top surface 604a of the surgical adjunct, the angled portion has a slope of about 30 degrees to about 60 degrees in reference to the top surface, wherein the proximal end chamfer comprises a proximal end with a width of about 0.27 inches to about 0.29 inches, wherein the proximal end chamfer 604c comprises a first angled side extension and a second angled side extension extending away from the distal end of the surgical adjunct to the proximal end of proximal end chamfer 604c, the first angled side extension and the second angled side extensions each have lengths of about 0.01 inches to about 0.40 inches when measured horizontally.

Clause D17: The surgical adjunct 604 of clause D1, further comprising a plurality of struts having a median strut thickness of about 0.025 mm to about 0.300 mm.

Clause D18: A method of making a surgical adjunct 604, comprising: selectively adding a plasticizer to a polyurethane foam to generate the surgical adjunct 604, wherein the surgical adjunct 604 has a glass transition temperature of about 0° C. to about 40° C.

Clause D19: The method of clause D18, wherein selectively adding the plasticizer comprises either soaking the polyurethane foam in in a solution comprising the plasticizer or adding the plasticizer on at least a portion of at least one surface of the polyurethane foam via direct deposition, and wherein a volumetric ratio of the polyurethane foam to the total volume of the surgical adjunct 604 is in a range of about 0.125 to about 0.325.

Clause D20: The method of clause D18, further comprising: laminating the polyurethane foam with a polydioxanone (PDO) film by: adhering one side of the polyurethane foam with a PDO film to create a partially sealed foam; subjecting the partially sealed foam to a temperature of about 105° C. to about 115° C.; subjecting the partially sealed foam to a pressure of about 5 kN; and letting the partially sealed foam stand at least thirty seconds; and subjecting the partially sealed foam to a temperature of about 35° C. to about 45° C.

Clause D21: A surgical adjunct 604, comprising: a polyurethane foam, and wherein a glass transition temperature of the surgical adjunct 604 is about 0° C. to about 40° C.

Clause D22: The surgical adjunct 604 of clause D21, wherein a volumetric ratio of the polyurethane foam to the total volume of the surgical adjunct 604 is in a range of about 0.125 to about 0.325.

Clause D23: The surgical adjunct 604 of clause D21, wherein the volumetric ratio of the polyurethane foam to total volume of the surgical adjunct is about 0.175 to about 0.225.

Clause D24: The surgical adjunct 604 of clause D21, wherein the volumetric ratio of the polyurethane foam to total volume of the surgical adjunct 604 is about 0.19 to about 0.21.

Clause D25: The surgical adjunct 604 of any of clauses D21 to D24, wherein the glass transition temperature of the surgical adjunct is about 7.5° C. to about 22.5° C.

Clause D26: The surgical adjunct 604 of any of clauses D21 to D24, further comprising a plasticizer added to the polyurethane foam, wherein the glass transition temperature of the surgical adjunct 604 is about 12.5° C. to about 17.5° C.

Clause D27: The surgical adjunct 604 of any of clauses D21 to D26, wherein a median pore size of the surgical adjunct 604 is about 0.025 to about 0.300 mm³.

Clause D28: The surgical adjunct 604 of any of clauses D21 to D27, further comprising a polydioxanone (PDO) film disposed on at least one surface of the polyurethane foam.

Clause D29: The surgical adjunct 604 of clause D28, wherein the PDO film has a thickness of about 20 μm to about 100 μm.

Clause D30: The surgical adjunct 604 of clause D26, wherein the plasticizer comprises at least one of a low molecular weight glycol, polyethylene glycol, polyvinylpyrrolidone, dibutyl sebacate, glyceryl triacetate, glyceryl behenate, hexanoic acid, decanoic acid, octadecanoic acid, boric ester, and a fatty acid.

Clause D31: The surgical adjunct 604 of clause D26, wherein the plasticizer comprises at least one fatty acid.

Clause D32: The surgical adjunct 604 of any of clauses D21 to D31, wherein the surgical adjunct 604 has a compression strength of about 30 to about 70 kPa.

Clause D33: The surgical adjunct 604 of any of clauses D21 to D31, wherein the surgical adjunct 604 has a compression strength of about 30 to about 50 kPa.

Clause D34: The surgical adjunct 604 of any of clauses D21 to D33, wherein the surgical adjunct 604 has a peak tensile strength of about 50 to about 150 kPa.

Clause D35: The surgical adjunct 604 of any of clauses D21 to D33, wherein the surgical adjunct 604 has a peak tensile strength of about 45 to about 85 kPa.

Clause D36: The surgical adjunct 604 of any of clauses D21 to D35, wherein the surgical adjunct 604 has a length of about 40 mm to about 80 mm; a width of about 8 mm to about 12 mm; and a height of about 2.5 mm to about 3.5 mm.

Clause D37: The surgical adjunct 604 of any of clauses D21 to D35, wherein the surgical adjunct 604 has a length of about 60 mm to about 70 mm or about 45 mm to about 55 mm; a width of about 9.75 mm to about 10.25 mm; and a height of about 2.85 mm to about 3.15 mm.

Clause D38: The surgical adjunct 604 of any of clauses D21 to D35, wherein the surgical adjunct 604 has: a length of about 66.04 mm to about 66.3 mm or about 21.12 mm to about 51.38 mm; a width of about 10.025 mm to about 10.035 mm; and a height of about 2.95 mm to about 3.05 mm.

Clause D39: A surgical staple cartridge assembly 106, comprising: a cartridge 200 having a length of about 80 mm to about 90 mm and a width of about 8.9 mm to about 14 mm; a surgical adjunct 604 disposed on the cartridge 200 comprising: a polyurethane foam comprising a volumetric ratio of the polyurethane foam to the total volume of the surgical adjunct 604 is in a range of about 0.125 to about 0.325, wherein a glass transition T_{g} temperature of the surgical adjunct 604 is about 0° C. to about 40° C., and wherein the surgical adjunct 604 having: a length of about 40 mm to about 80 mm; a width of about 8 mm to about 12 mm; and a height of about 2.5 mm to about 3.5 mm.

Clause D40: The surgical cartridge assembly 106 of clause D39, wherein the surgical adjunct 604 further comprises a plasticizer added to the polyurethane foam.

Clause D41: The surgical cartridge assembly 106 of clauses D39 or D40, wherein the cartridge 200 comprises at least two approximately parallel sets of staple cavities 212, 214 spaced apart by a longitudinal slot 210, each staple cavity 212, 214 having: an approximately parallel orientation with the longitudinal slot 210; a mouth-like shape; a maximum length of about 0.122 to about 0.124 inches; and a maximum width of about 0.023 inches to about 0.027 inches.

Clause D42: The surgical cartridge assembly 106 of clause D39, wherein at least centers of two adjacent staple cavities 212 in a row 212a are spaced apart by about 0.158 inches.

Clause D43: The surgical cartridge assembly 106 of clause D39, wherein the surgical adjunct 604 comprises two parallel portions 806a, 806b separated by a slot 808.

Clause D44: The surgical cartridge assembly 106 of clause D39, wherein the surgical adjunct 604 comprises two parallel portions 806a, 806b separated by at least one slit 808, at least one bridge 802 connecting the two parallel portions 806a, 806b across the at least one slit 808.

Clause D45: The surgical cartridge assembly 106 of clause D44, wherein the at least one bridge 802 comprises a length of about 0.035 inches to about 0.046 inches.

Clause D46. The surgical cartridge assembly 106 of any of clauses D39 to D45, wherein an adhesive is disposed between the surgical adjunct 604 and the cartridge 200 and is configured to adhere the surgical adjunct to the cartridge.

Clause D47: The surgical cartridge assembly 106 of clause D46, further comprising about 100 mg to about 120 mg of adhesive between the cartridge 200 and the surgical adjunct 604.

Clause D48: The surgical cartridge assembly 106 of any of clauses D39 to D47, wherein the cartridge 200 comprises: a deck 206 comprising at least one raised surface 804 along lateral and/or distal sides of the cartridge 200 and configured to align the surgical adjunct 604; and an atraumatic shaped distal end DC.

Clause D49: The surgical cartridge assembly 106 of any of clauses D39 to D47, wherein the cartridge 200 comprises a substantially flat deck 206.

Clause D50: The surgical cartridge assembly 106 of any of clauses D39 to D48, wherein the cartridge 200 comprises a plurality of raised surfaces positioned at each distal end and proximal end of each staple cavity 212, 214, and wherein a raised surface corresponding to a distal end of a staple cavity 212, 214 forms a combined raised surface with a raised surface corresponding to a proximal end of an adjacent staple cavity 212, 214.

Clause D51: The surgical cartridge assembly 106 of any of clauses D39 to D50, wherein the cartridge 200 comprises a plurality of lowered surfaces that are lower than the deck 206, and wherein at least one lowered surface is positioned along at least one lateral side of a staple cavity 212, 214.

Clause E1: A method for embedding medical additives into a bioabsorbable material, the method comprising: chemically reacting one or more functional groups of a bioabsorbable material with one or more medical additives, wherein the bioabsorbable material is configured to be placed inside a body of a human, and wherein the one or more medical additives are configured to remain chemically bonded to a foam structure of the bioabsorbable material until a biodegradation of the bioabsorbable material.

Clause E2: The method of clause E1, wherein the bioabsorbable material comprises polyurethane.

Clause E3: The method of clause E2, wherein the bioabsorbable material is a reaction product of a polyol and an isocyanate.

Clause E4: The method of clause E3, wherein the one or more medical additives react with the polyol.

Clause E5: The method of clause E1, wherein the bioabsorbable material is configured to be used with a surgical staple cartridge configured to repair one or more parts of the body of the human.

Clause E6: The method of clause E1, wherein the one or more functional groups comprise isocyanate groups.

Clause E7: The method of clause E6, wherein the one or more medical additives comprise one or more polydrugs.

Clause E8: The method of clause E7, wherein chemically reacting the one or more functional groups of the bioabsorbable material with the one or more medical additives comprises endcapping the isocyanate groups with the one or more polydrugs.

Clause E9: The method of clause E7, wherein the polydrugs treat one or more of pain, wound healing, tissue growth, infection reduction, immunosuppression, radiotherapy, or combinations thereof.

Clause E10: The method of clause E1, wherein the one or more functional groups comprise polyol hydroxyl groups.

Clause E11: The method of clause E10, wherein chemically reacting the one or more functional groups of the bioabsorbable material with the one or more medical additives comprises functionalizing the polyol hydroxyl groups with the one or more medical additives.

Clause E12: The method of clause E11, wherein the one or more medical additives comprise one or more medications that treat one or more of pain, wound healing, tissue growth, infection reduction, immunosuppression, radiotherapy, or combinations thereof.

Clause E13: The method of clause E1, wherein chemically reacting the one or more functional groups of the bioabsorbable material with the one or more medical additives comprises modifying the one or more functional groups via one or more reactive surfactants.

Clause E14: A method for embedding medical additives into a bioabsorbable material, the method comprising: dissolving one or more water soluble medical additives into an aqueous phase; and incorporating the aqueous phase into a foam network of a bioabsorbable material, wherein the bioabsorbable material is configured to be placed inside a body of a human.

Clause E15: The method of clause E14, wherein the one or more water soluble medical additives are configured to be released into the body of the human over a period of time during a biodegradation of the bioabsorbable material.

Clause E16: The method of clause E14, wherein dissolving the one or more water soluble medical additives into the aqueous phase comprises generating a colloidal suspension comprising one or more silver nanoparticles.

Clause E17: The method of clause E14, wherein the one or more water soluble medical additives comprise at least one of drugs or vitamins.

Clause E18: A bioabsorbable material configured to be used with a surgical staple cartridge configured to repair one or more parts of a body of a human, the bioabsorbable material comprising: one or more isocyanate groups; and one or more medical additives chemically bonded to the one or more isocyanate groups, wherein the one or more medical additives comprise polydrugs.

Clause E19: The bioabsorbable material of clause E18, wherein the one or more medical additives are configured to remain chemically bonded to a foam structure of the bioabsorbable material until a biodegradation of the bioabsorbable material.

Clause E20: The bioabsorbable material of clause E18, wherein the polydrugs treat one or more of pain, wound healing, tissue growth, infection reduction, immunosuppression, radiotherapy, or combinations thereof.

Clause E21: A method for embedding medical additives into a bioabsorbable material, the method comprising: chemically reacting one or more functional groups of a bioabsorbable material with one or more medical additives, wherein the bioabsorbable material is configured to be placed inside a body of a human, and wherein the one or more medical additives are configured to remain chemically bonded to a foam structure of the bioabsorbable material until a biodegradation of the bioabsorbable material.

Clause E22: The method of clause E21, wherein the bioabsorbable material comprises polyurethane.

Clause E23: The method of any of clauses E21-E22, wherein the bioabsorbable material is a reaction product of a polyol and an isocyanate.

Clause E24: The method of clause E23, wherein the one or more medical additives react with the polyol.

Clause E25: The method of any of clauses E21-E24, wherein the bioabsorbable material is configured to be used with a surgical staple cartridge configured to repair one or more parts of the body of the human.

Clause E26: The method of any of clauses E21-E25, wherein the one or more functional groups comprise isocyanate groups.

Clause E27: The method of any of clauses E21-E26, wherein the one or more medical additives comprise one or more polydrugs.

Clause E28: The method of clause E27, wherein chemically reacting the one or more functional groups of the bioabsorbable material with the one or more medical additives comprises endcapping the isocyanate groups with the one or more polydrugs.

Clause E29: The method of any of clauses E27-E28, wherein the polydrugs treat one or more of pain, wound healing, tissue growth, infection reduction, or combinations thereof.

Clause E30: The method of any of clauses E21-E25, wherein the one or more functional groups comprise polyol hydroxyl groups.

Clause E31: The method of clause E30, wherein chemically reacting the one or more functional groups of the bioabsorbable material with the one or more medical additives comprises functionalizing the polyol hydroxyl groups with the one or more medical additives.

Clause E32: The method of any of clauses E21-E31, wherein the one or more medical additives comprise one or more medications that treat one or more of pain, wound healing, tissue growth, infection reduction, or combinations thereof.

Clause E33: The method of any of clauses E21-E32, wherein chemically reacting the one or more functional groups of the bioabsorbable material with the one or more medical additives comprises modifying the one or more functional groups via one or more reactive surfactants.

Clause E34: The method of any of clauses E21-E33, wherein chemically reacting the one or more functional groups of the bioabsorbable material with the one or more medical additives allows for a steady release profile of the one or more medical additives from the foam structure during the biodegradation of the bioabsorbable material.

Clause E35: The method of any of clauses E21-E34, wherein the one or more medical additives treat one or more of pain, wound healing, tissue growth, infection reduction, or combinations thereof.

Clause F1: A surgical adjunct 604, comprising: a polyurethane foam comprising a volumetric ratio of the polyurethane foam to the total volume of the surgical adjunct 604 is in a range of about 0.125 to about 0.325; and at least one hydrophobicity additive comprising at least one ceramic nanoparticle, at least one fatty acid, at least one ionic liquid, at least one long chain surfactant, at least one enteric coating, at least one photocurable resin, or combinations thereof.

Clause F2: The surgical adjunct 604 of Clause F1, wherein the at least one hydrophobicity additive comprises the at least one fatty acid comprising oleic acid, decanoic acid, hexanoic acid, dodecanoic acid, or combinations thereof.

Clause F3: The surgical adjunct 604 of Clause F2, wherein the at least one fatty acid comprises oleic acid.

Clause F4: The surgical adjunct 604 of Clause F1, wherein the at least one hydrophobicity additive comprises the at least one ceramic nanoparticle.

Clause F5: The surgical adjunct 604 of Clause F4, wherein the at least one ceramic nanoparticle comprises calcium phosphate.

Clause F6: The surgical adjunct 604 of Clause F1, wherein the at least one hydrophobicity additive comprises the at least one long chain surfactant.

Clause F7: The surgical adjunct 604 of Clause F1, wherein the at least one hydrophobicity additive is a hydrophobic coating disposed on the polyurethane foam.

Clause F8: The surgical adjunct 604 of Clause F7, wherein the hydrophobic coating comprises the at least one fatty acid, the at least one long chain surfactant, the at least one enteric coating, the at least one photocurable resin, or combinations thereof.

Clause F9: A surgical adjunct 604, comprising: a polyurethane foam comprising a volumetric ratio of the polyurethane foam to the total volume of the surgical adjunct 604 is in a range of about 0.125 to about 0.325, wherein the polyurethane foam comprises a hydrophobic surface pattern.

Clause F10: The surgical adjunct 604 of Clause F9, wherein the surface pattern comprises at least one repeating pattern with a pitch that is less than about 0.005 centimeters.

Clause F11: A surgical adjunct 604, comprising: a polyurethane foam comprising a volumetric ratio of the polyurethane foam to the total volume of the surgical adjunct 604 is in a range of about 0.125 to about 0.325; and a moisture barrier dividing the polyurethane foam into at least two sections and configured to prevent fluid ingress.

Clause F12: The surgical adjunct 604 of Clause F11, wherein the moisture barrier comprises a film comprising an aliphatic polyester.

Clause F13: The surgical adjunct 604 of Clause F11, wherein the moisture barrier comprises a matrix splitting the polyurethane foam.

Clause F14: The surgical adjunct 604 of Clause F11, wherein the moisture barrier comprises one or more poly(p-dioxanone) coated sutures interlaced within the polyurethane foam.

Clause F15: A surgical adjunct 604, comprising: a polyurethane foam comprising a plurality of pores configured to prevent or reduce fluid ingress into the polyurethane foam.

Clause F16: The surgical adjunct 604 of Clause F15, wherein a volumetric ratio of the polyurethane foam to the total volume of the surgical adjunct 604 is in a range of about 0.125 to about 0.325.

Clause F17: The surgical adjunct 604 of Clause F15, wherein the plurality of pores form a gradient or double gradient based on an average diameter and with respect to a first side of the polyurethane foam.

Clause F18: The surgical adjunct 604 of Clause F15, wherein at least some of the plurality of pores are aligned in a first direction to direct fluid ingress within the polyurethane foam in at least a first direction and a second direction opposite the first direction.

Clause F19: The surgical adjunct 604 of Clause F18, wherein at least some of the plurality of pores are closed cell pores configured to prevent fluid ingress.

Clause F20: The surgical adjunct 604 of Clause F15, wherein at least some of the plurality of pores are close celled pores configured to prevent fluid ingress.

Clause F21: A surgical adjunct 604, comprising: a polyurethane foam comprising a volumetric ratio of the polyurethane foam to the total volume of the surgical adjunct 604 is in a range of about 0.125 to about 0.325.

Clause F22: The surgical adjunct 604 of Clause F21, further comprising: at least one hydrophobicity additive comprising at least one ceramic nanoparticle, at least one fatty acid, at least one ionic liquid, at least one long chain surfactant, at least one enteric coating, at least one photocurable resin, or combinations thereof.

Clause F23: The surgical adjunct 604 of Clause F22, wherein the at least one hydrophobicity additive comprises the at least one fatty acid comprising oleic acid, decanoic acid, hexanoic acid, dodecanoic acid, or combinations thereof.

Clause F24: The surgical adjunct 604 of Clause F23, wherein the at least one fatty acid comprises oleic acid, decanoic acid, hexanoic acid, dodecanoic acid, or combinations thereof.

Clause F25: The surgical adjunct 604 of Clauses F22 to F24, wherein the at least one hydrophobicity additive comprises the at least one ceramic nanoparticle.

Clause F26: The surgical adjunct 604 of Clauses F22 to F25, wherein the at least one ceramic nanoparticle comprises calcium phosphate.

Clause F27: The surgical adjunct 604 of Clauses F22 to F26, wherein the at least one hydrophobicity additive is a hydrophobic coating disposed on the polyurethane foam, the hydrophobic coating comprising the at least one fatty acid, the at least one long chain surfactant, the at least one enteric coating, the at least one photocurable resin, or combinations thereof.

Clause F28: The surgical adjunct 604 of Clause F21 to F27, wherein the polyurethane foam comprises a hydrophobic surface pattern.

Clause F29: The surgical adjunct 604 of Clause F28, wherein the surface pattern comprises at least one repeating pattern with a pitch that is less than about 0.005 centimeters.

Clause F30: The surgical adjunct 604 of Clauses F21 to F29 further comprising a moisture barrier dividing the polyurethane foam into at least two sections and configured to prevent fluid ingress.

Clause F31: The surgical adjunct 604 of Clause F30, wherein the moisture barrier comprises a film comprising an aliphatic polyester.

Clause F32: The surgical adjunct 604 of Clauses F30 or F31, wherein the moisture barrier comprises a matrix splitting the polyurethane foam.

Clause F33: The surgical adjunct 604 of Clauses F30 to F32, wherein the moisture barrier comprises one or more poly(p-dioxanone) coated sutures interlaced within the polyurethane foam.

Clause F34: The surgical adjunct 604 of Clauses F21 to F33, wherein the polyurethane foam comprises a plurality of pores configured to prevent or reduce fluid ingress into the polyurethane foam.

Clause F35: The surgical adjunct 604 of Clause F21, wherein the plurality of pores (i) have an average diameter of the plurality of pores that form a gradient or double gradient with respect to a first side of the polyurethane foam, (ii) are aligned in one direction to direct fluid ingress within the polyurethane foam, (iii) comprise closed cell pores, or combinations thereof.

Clause G1: A surgical adjunct 604, comprising: a polyurethane foam 902 comprising a volumetric ratio of the polyurethane foam 902 to the total volume of the surgical adjunct 604 is in a range of about 0.125 to about 0.325; and a film 904 disposed on at least one surface of the polyurethane foam.

Clause G2: The surgical adjunct 604 of clause G1, wherein the film 904 comprises polyurethane having a higher density than the polyurethane foam.

Clause G3: The surgical adjunct 604 of clause G1, wherein the film 904 has a thickness FT of about 0.0003 inches to about 0.010 inches and has pore diameters of about 0.0005 inches to about 0.005 inches.

Clause G4: The surgical adjunct 604 of clause G1, wherein the film 904 is laminated on the at least one surface of the polyurethane foam 902 and comprises an absorbable material selected from the group consisting of polydioxanone (PDO), poly(delta-gluconolactone) (PGL-1), poly(glycolide/l-lactide) (PGL-2), polyglycolic acid (PGA), a glycolide and epsilon caprolactone copolymer (PGCL), a glycolide and l-lactide copolymer, urethane, polycaprolactone (PCL), polyglactin 370 (PG-370), polyglactin 185 (PG-185), or combinations thereof.

Clause G5: The surgical adjunct 604 of clause G4, wherein the absorbable material comprises polyglactin 370 or polyglactin 185.

Clause G6: The surgical adjunct 604 of clause G4, wherein the absorbable material comprises poly(glycolide/l-lactide).

Clause G7: The surgical adjunct 604 of clause G4, wherein the film 904 has a thickness FT of about 0.0003 inches to about 0.003 inches.

Clause G8: The surgical adjunct 604 of clause G1, wherein the film 904 is attached to the at least one surface of the polyurethane foam 902 with an adhesive 906.

Clause G9: The surgical adjunct 604 of clause G8, wherein the adhesive 906 comprises polyvinylpyrrolidone.

Clause G10: The surgical adjunct 604 of clause G1, wherein the surgical adjunct 604 is attached to the deck with a bioabsorbable adhesive.

Clause G11: A method for making the surgical adjunct 604 of clause G1, comprising: providing the polyurethane foam 902; disposing at least one volatile solvent on at least a portion of at least one surface of the polyurethane foam; disposing the film 904 on the at least one volatile solvent 906; and dissolving a least a portion of the polyurethane foam and the film 904 with the at least one volatile solvent to join the polyurethane foam and the film.

Clause G12: A method for making the surgical adjunct 604 of clause G1, comprising: providing the polyurethane foam 902; disposing a reactive adhesive on at least a portion of the film 904, at least a portion of the polyurethane foam, or both; and disposing the film on the at least polyurethane foam 902 such that the reactive adhesive is sandwiched between the polyurethane foam and the film 904.

Clause G13: The method of clause G12, wherein disposing the reactive adhesive comprises applying the adhesive via at least one application process selected from the group consisting of inkjet printing, direct deposition, thermal spraying, cold dynamic spraying, cold spraying, electro spraying, ultrasonic spray coating, dip coating, screen printing, spin coating, or combinations thereof.

Clause G14: A method for making the surgical adjunct 604 of clause G1, comprising: provide a polyurethane foam precursor cured to its gel point to form a partially cured polyurethane foam; disposing the film 904 on the partially cured polyurethane foam on at least a portion of one surface; and finalize curing the partially cured polyurethane foam with the film to form the surgical adjunct 604.

Clause G15: A method for making the surgical adjunct 604 of clause G1, comprising: providing the polyurethane foam 902; applying the film to the polyurethane foam 902 via a direct deposition method to create the surgical adjunct 904.

Clause G16: The method of clause G15, wherein the direct deposition method comprises at least one process selected from the group consisting of stereolithography, lithography, holographic printing, inkjet printing, direct deposition, thermal spraying, cold dynamic spraying, cold spraying, electro spraying, ultrasonic spray coating, dip coating, screen printing, spin coating, or combinations thereof.

Clause G17: A cartridge for surgical stapling, comprising: a deck 206 comprising at least one post 1104 extending away from the deck 206, wherein the at least one post having a first diameter PDT at a top 1106 of the at least one post 1104 that is greater than a second diameter PDB at the bottom 1108 of the at least one post 1104; a first film 904 disposed on the deck 206 and at least partially surrounding the at least one post with gap 1110 between the top 1106 of the at least one post and the film; and a surgical adjunct 604 disposed on the first film 904 and at least partially surrounding the at least one post 1104, the surgical adjunct 604 comprising a polyurethane foam 902 with a volumetric ratio of the polyurethane foam 902 to the total volume of the surgical adjunct 604 is in a range of about 0.125 to about 0.325.

Clause G18: The cartridge of clause G17, wherein the first film 904 comprises a perforated pattern at least partially surrounding or covering one or more staple slots 1112.

Clause G19: The cartridge of clause G17, further comprising a second film 904 disposed on polyurethane foam, wherein the first film 904 has a first melting point that is lower than a second melting point of the polyurethane foam 902.

Clause G20: A method of making the cartridge of clause G17, the method comprising: providing the deck 206 with the at least one post; disposing the film 904 on the deck and at least partially surrounding the at least one post 1104; applying heat to the at least one post 1104 to form the top 1106 with the first diameter PDT and the bottom 1108 with the second diameter PDB such that the top of the at least one post at least partially overlaps the film 904; and disposing the surgical adjunct 604 on the film to at least partially surround the at least one post 1104.

Clause G21: A stapling assembly 600 for surgical stapling, comprising: a deck 206; and a surgical adjunct 604 disposed on deck 604 and comprising a polyurethane foam 902 with a volumetric ratio of the polyurethane foam 902 to the total volume of the surgical adjunct 604 is in a range of about 0.125 to about 0.325.

Clause G22: The stapling assembly of clause G21, wherein the surgical adjunct 604 comprises a film 904 disposed on at least one surface of the polyurethane foam 902.

Clause G23: The stapling assembly of clause G22, wherein the film 904 comprises polyurethane 902 having a higher density than the polyurethane foam 902.

Clause G24: The stapling assembly of clause G22, wherein the film 904 has a thickness FT of about 0.0003 inches to about 0.010 inches and has pore diameters of about 0.0005 inches to about 0.005 inches.

Clause G25: The stapling assembly of clause G22, wherein the film 904 is laminated on the at least one surface of the polyurethane foam and comprises an absorbable material selected from the group consisting of polydioxanone (PDO), poly(delta-gluconolactone) (PGL-1), poly(glycolide/l-lactide) (PGL-2), polyglycolic acid (PGA), a glycolide and epsilon caprolactone copolymer (PGCL), a glycolide and l-lactide copolymer, urethane, polycaprolactone (PCL), polyglactin 370 (PG-370), polyglactin 185 (PG-185), or combinations thereof.

Clause G26: The stapling assembly of clause G25, wherein the film 904 has a thickness FT of about 0.0003 inches to about 0.003 inches.

Clause G27: The stapling assembly of clause G25 or G26, wherein the film 904 is attached to the at least one surface of the polyurethane foam with an adhesive.

Clause G28: The stapling assembly of clause G21, wherein the absorbable material comprises polyglactin 370.

Clause G29: The stapling assembly of clause G21, wherein the surgical adjunct 604 further comprises an additional polyurethane foam 902 attached to the polyurethane foam 902, wherein the polyurethane foam has a first density gradient and the additional polyurethane foam 902 has a second density gradient that is approximately opposite of the first density gradient.

Clause G30: The stapling assembly of clause G21, wherein: the deck 206 comprises a surface and at least one post 1104 extending away from the surface, the at least one post 1104 having a top 1106 with a first diameter PDT and a bottom 1108 with a second diameter PDB that is less than the first diameter PDT, the film 904 is disposed on the surface and at least partially surrounds the at least one post 1104 with a gap 1110 between the top 1106 of the at least one post 1104 and the film 904; and the surgical adjunct 604 is disposed on the film 904 and at least partially surrounding the at least one post.

Clause G31: The stapling assembly of clause G30, wherein the film 904 comprises a perforated pattern surrounding one or more staple slots 1112.

Clause G32: A method for making a surgical tool 200, comprising: providing a polyurethane foam 902 comprising a volumetric ratio of the polyurethane foam 902 to the total volume of the surgical adjunct 604 is in a range of about 0.125 to about 0.325; and disposing or forming a film 904 on the polyurethane foam 902.

Clause G33: The method of clause G32, comprising: disposing at least one volatile solvent 906 on at least a portion of at least one surface of the polyurethane foam 902; disposing the film 904 on the at least one volatile solvent 906; and dissolving a least a portion of the polyurethane foam 902 and the film 904 with the at least one volatile solvent to join the polyurethane foam 902 and the film 904.

Clause G34. The method of clause G32, comprising: providing the polyurethane foam 902; disposing a reactive adhesive 906 on at least a portion of the film 904, at least a portion of the polyurethane foam 902, or both; and disposing the film on the at least polyurethane foam 902 such that the reactive adhesive 906 is sandwiched between the polyurethane foam 902 and the film 904.

Clause G35: The method of clause G32, comprising: providing a cartridge 200 comprising a deck with at least one post extending from the deck; disposing the film 904 on the deck and at least partially surrounding the at least one post 1104; applying heat to the at least one post 1104 to form the top 1106 with the first diameter PDT and the bottom 1108 with the second diameter PDB such that the top of the at least one post at least partially overlaps the film 904; and disposing the surgical adjunct 604 on the film 904 to at least partially surround the at least one post 1104.

Clause H1: A method of attaching an adjunct to a surgical staple cartridge using a biocompatible adhesive, the method comprising: depositing a biocompatible adhesive onto a top surface of a surgical staple cartridge; and attaching an adjunct to the biocompatible adhesive, wherein the biocompatible adhesive comprises a hydrophobic component configured to reduce a sensitivity of the biocompatible adhesive to moisture and temperature.

Clause H2: The method of clause H1, wherein the hydrophobic component comprises a wax having a melting temperature of at least approximately 60 degrees Celsius.

Clause H3: The method of clause H2, wherein the wax comprises candelilla wax, microcrystalline wax, montan wax, white wax, or combinations thereof.

Clause H4: The method of clause H1, wherein the hydrophobic component comprises polyurethane.

Clause H5: The method of clause H1, wherein depositing the biocompatible adhesive onto the top surface of the surgical staple cartridge is conducted via at least one of direct deposition, spray coating, spin coating, dip coating, ultrasonic spray coating, screen printing, electrospinning, electrospray, thermal spray, or combinations thereof.

Clause H6: A method of attaching an adjunct to a surgical staple cartridge using a biocompatible adhesive, the method comprising: depositing a biocompatible adhesive onto a top surface of a surgical staple cartridge; and attaching an adjunct to the biocompatible adhesive, wherein the biocompatible adhesive is configured to selectively detach from the surgical staple cartridge based on a pH of an environment surrounding the adjunct.

Clause H7: The method of clause H6, wherein the biocompatible adhesive comprises an enteric material.

Clause H8: The method of clause H7, wherein the enteric material comprises at least one of a cellulose derivative or a methacrylate copolymer.

Clause H9: The method of clause H7, wherein the enteric material is configured to adhere to the surgical staple cartridge when the pH of the environment is less than approximately 6.0.

Clause H10: The method of clause H7, wherein the enteric material is configured to detach from the surgical staple cartridge when the pH of the environment is at least approximately 6.0.

Clause H11: The method of clause H6, wherein the biocompatible adhesive comprises a stimuli responsive material.

Clause H12: The method of clause H11, wherein the stimuli responsive material comprises at least one of an arylboronic acid, a styrylpyrene, an o-nitrobenzyl, a coumarin, or a polyol.

Clause H13: The method of clause H11, wherein the stimuli responsive material is configured to reduce a sensitivity of the biocompatible adhesive to moisture and temperature.

Clause H14: The method of clause H11, wherein the biocompatible adhesive is further configured to selectively detach from the surgical staple cartridge based on exposure to one or more wavelengths of light.

Clause H15: The method of clause H14, wherein the one or more wavelengths are between approximately 300 to 475 nm.

Clause H16: The method of clause H11, wherein the stimuli responsive material comprises at least one of a redox-responsive material, a photo-responsive material, or a mechano-responsive material.

Clause H17: The method of clause H6, wherein depositing the biocompatible adhesive onto the top surface of the surgical staple cartridge is conducted via at least one of direct deposition, spray coating, spin coating, dip coating, ultrasonic spray coating, screen printing, electrospinning, electrospray, thermal spray, or combinations thereof.

Clause H18: A method of attaching an adjunct to a surgical staple cartridge using a biocompatible adhesive, the method comprising: depositing a biocompatible adhesive onto a top surface of a surgical staple cartridge; and attaching an adjunct to the biocompatible adhesive, wherein the biocompatible adhesive comprises a component configured to reduce a sensitivity of the biocompatible adhesive to moisture and temperature.

Clause H19: The method of clause H18, wherein the component comprises a hydrogel network configured to exhibit an adhesive strength based on one or more chemical interactions.

Clause H20: The method of clause H19, wherein the one or more chemical interactions comprise at least one of hydrogen bonding or dipole-dipole interactions.

Clause H21: A method of attaching an adjunct to a surgical staple cartridge using a biocompatible adhesive, the method comprising: depositing a biocompatible adhesive onto a top surface of a surgical staple cartridge; and attaching an adjunct to the biocompatible adhesive, wherein the biocompatible adhesive comprises a component configured to reduce a sensitivity of the biocompatible adhesive to moisture and temperature.

Clause H22: The method of clause H21, wherein the component comprises a hydrophobic component.

Clause H23: The method of any of clauses H21-H22, wherein the component comprises a wax having a melting temperature of at least approximately 60 degrees Celsius.

Clause H24: The method of any of clauses H21-H23, wherein the wax comprises candelilla wax, microcrystalline wax, montan wax, white wax, or combinations thereof.

Clause H25: The method of any of clauses H21-H22, wherein the component comprises polyurethane.

Clause H26: The method of clause H21, wherein the biocompatible adhesive is configured to selectively detach from the surgical staple cartridge based on a pH of an environment surrounding the adjunct.

Clause H27: The method of any of clauses H21 and H26, wherein the biocompatible adhesive comprises an enteric material.

Clause H28: The method of clause H27, wherein the enteric material comprises at least one of a cellulose derivative or a methacrylate copolymer.

Clause H29: The method of any of clauses H27-H28, wherein the enteric material is configured to adhere to the surgical staple cartridge when the pH of the environment is less than approximately 6.0.

Clause H30: The method of any of clauses H27-H29, wherein the enteric material is configured to detach from the surgical staple cartridge when the pH of the environment is at least approximately 6.0.

Clause H31: The method of any of clauses H21 and H26, wherein the biocompatible adhesive comprises a stimuli responsive material.

Clause H32: The method of clause H31, wherein the stimuli responsive material comprises at least one of an arylboronic acid, a styrylpyrene, an o-nitrobenzyl, a coumarin, or a polyol.

Clause H33: The method of any of clauses H31-H32, wherein the biocompatible adhesive is further configured to selectively detach from the surgical staple cartridge based on exposure to one or more wavelengths of light.

Clause H34: The method of clause H33, wherein the one or more wavelengths are between approximately 300 to 475 nm.

Clause H35: The method of any of clauses H31-H34, wherein the stimuli responsive material comprises at least one of a redox-responsive material, a photo-responsive material, or a mechano-responsive material.

Clause H36: The method of clause H21, wherein the component comprises a hydrogel network configured to exhibit an adhesive strength based on one or more chemical interactions.

Clause H37: The method of clause H36, wherein the one or more chemical interactions comprise at least one of hydrogen bonding or dipole-dipole interactions.

Clause H38: The method of any of clauses H36-H37, wherein the hydrogel network utilizes at least one of covalent or ionic crosslinks.

Clause H39: The method of any of clauses H36-H38, wherein the hydrogel network causes the biocompatible adhesive to be released into an environment surrounding the adjunct based on at least one of water swelling or counterion exchange.

Clause H40: The method of any of clauses H36-H39, wherein the hydrogel network comprises at least one of carrageenan, alginate, a polysaccharide, or cellulose.

Clause H41: The method of any of clauses H36-H40, wherein the hydrogel network is configured to deliver one or more therapeutic agents into an environment surrounding the adjunct.

Clause H42: The method of any of clauses H21-H41, wherein depositing the biocompatible adhesive onto the top surface of the surgical staple cartridge is conducted via at least one of direct deposition, spray coating, spin coating, dip coating, ultrasonic spray coating, screen printing, electrospinning, electrospray, thermal spray, or combinations thereof.

Clause H43: The method of any of clauses H21-H42, wherein depositing the biocompatible adhesive onto the top surface of the surgical staple cartridge is conducted by at least one of a batch process or a continuous process.

What is claimed is:

1. A surgical adjunct 604, comprising:
a polyurethane foam comprising a volumetric ratio of the polyurethane foam to the total volume of the surgical adjunct 604 is in a range of about 0.125 to about 0.325, and
wherein a glass transition temperature of the surgical adjunct 604 is about 0° C. to about 40° C.

2. The surgical adjunct 604 of claim 1, wherein the volumetric ratio of the polyurethane foam to total volume of the surgical adjunct is about 0.175 to about 0.225.

3. The surgical adjunct 604 of claim 1, wherein the volumetric ratio of the polyurethane foam to total volume of the surgical adjunct is about 0.19 to about 0.21

4. The surgical adjunct 604 of claim 1, wherein the glass transition temperature of the surgical adjunct is about 7.5° C. to about 22.5° C.

5. The surgical adjunct 604 of claim 1, wherein the glass transition temperature of the surgical adjunct 604 is about 12.5° C. to about 17.5° C.

6. The surgical adjunct 604 of claim 1, wherein a median pore size of the surgical adjunct 604 is about 0.025 to about 0.300 mm³.

7. The surgical adjunct 604 of claim 1, further comprising a polydioxanone (PDO) film disposed on at least one surface of the polyurethane foam.

8. The surgical adjunct 604 of claim 7, wherein the PDO film has a thickness of about 20 μm to about 100 μm.

9. The surgical adjunct 604 of claim 1, further comprising a plasticizer added to the polyurethane foam.

10. The surgical adjunct 604 of claim 9, wherein the plasticizer comprises at least one of a low molecular weight glycol, polyethylene glycol, polyvinylpyrrolidone, dibutyl sebacate, glyceryl triacetate, glyceryl behenate, hexanoic acid, decanoic acid, octadecanoic acid, boric ester and a fatty acid.

11. The surgical adjunct 604 of claim 9, wherein the plasticizer comprises at least one fatty acid.

12. The surgical adjunct 604 of claim 1, wherein the surgical adjunct 604 has a compression strength of about 30 kPa to about 70 kPa.

13. The surgical adjunct 604 of claim 12, wherein the compression strength is about 30 to about 60 kPa.

14. The surgical adjunct 604 of claim 1, wherein the surgical adjunct 604 has a peak tensile strength of about 50 to about 150 kPa.

15. The surgical adjunct 604 of claim 1, wherein the surgical adjunct 604 has:
a length of about 40 mm to about 80 mm;
a width of about 8 mm to about 12 mm; and
a height of about 2.5 mm to about 3.5 mm.

16. The surgical adjunct 604 of claim 15, wherein the surgical adjunct 604 has a distal end chamfer 604b and a proximal end chamfer 604c,
wherein the distal end chamfer comprises a vertical portion extending from a bottom of the surgical adjunct 604, the vertical portion has a height of about 0.009 inches to about 0.029 inches,
wherein the distal end chamfer comprises an angled portion extending from the vertical portion to a top surface 604a of the surgical adjunct, the angled portion has a slope of about 30 degrees to about 60 degrees in reference to the top surface,
wherein the proximal end chamfer comprises a proximal end with a width of about 0.27 inches to about 0.29 inches,
wherein the proximal end chamfer 604c comprises a first angled side extension and a second angled side extension extending away from the distal end of the surgical adjunct to the proximal end of proximal end chamfer 604c, the first angled side extension and the second angled side extensions each have lengths of about 0.01 inches to about 0.40 inches when measured horizontally.

17. The surgical adjunct 604 of claim 1, further comprising a plurality of struts having a median strut thickness of about 0.025 mm to about 0.300 mm.

18. A method of making a surgical adjunct 604, comprising:
selectively adding a plasticizer to a polyurethane foam to generate the surgical adjunct 604, wherein the surgical adjunct 604 has a glass transition temperature of about 0° C. to about 40° C.

19. The method of claim 18, wherein selectively adding the plasticizer comprises either soaking the polyurethane foam in in a solution comprising the plasticizer or adding the plasticizer on at least a portion of at least one surface of the polyurethane foam via direct deposition, and wherein a volumetric ratio of the polyurethane foam to the total volume of the surgical adjunct 604 is in a range of about 0.125 to about 0.325.

20. The method of claim 18, further comprising:
laminating the polyurethane foam with a polydioxanone (PDO) film by:
adhering one side of the polyurethane foam with a PDO film to create a partially sealed foam;
subjecting the partially sealed foam to a temperature of about 105° C. to about 115° C.;
subjecting the partially sealed foam to a pressure of about 5 kN;
letting the partially sealed foam stand at least thirty seconds; and
subjecting the partially sealed foam to a temperature of about 35° C. to about 45° C.
